(19) United States
(12) Patent Application Publication
(10) Pub. No.: US 2013/0303500 A1
(43) Pub. Date: Nov. 14, 2013

(54) COMPOUNDS AND METHODS FOR TREATING NEOPLASIA

(71) Applicants: Konstantinos Alevizopoulos, Giez (CH); Theodora Calogeropoulou, Athens (GR)

(72) Inventors: Konstantinos Alevizopoulos, Giez (CH); Theodora Calogeropoulou, Athens (GR)

(73) Assignee: Medexis S.A., Athens (GR)

(21) Appl. No.: 13/813,398

(22) PCT Filed: Jul. 29, 2011
(86) PCT No.: PCT/EP2011/063169

§ 371 Date: Jul. 29, 2013

Related U.S. Application Data
(60) Provisional application No. 61/369,568, filed on Jul. 30, 2010.

(51) Int. Cl. C07J 43/00

(52) U.S. Cl. CPC C07J 43/003
USPC 514/176; 540/106; 435/375

(57) ABSTRACT

The invention features compounds, pharmaceutical compositions and methods useful for the treatment of neoplasia. In particular embodiments, the compounds of the invention are useful for the treatment of multidrug resistant neoplasia.

### RELATED APPLICATION

This application claims the benefit of U.S. provisional patent application Ser. No. 61/369,568, filed Jul. 30, 2010, the entire disclosure of which is incorporated herein by this reference.

### BACKGROUND OF THE INVENTION

Approximately one million Americans are diagnosed with neoplasia every year, and about half a million people in the United States die of the disease annually. Although improvements in neoplasia detection, diagnosis, and treatment have increased the survival rate for many types of neoplasia, only about 60 percent of people diagnosed with neoplasia are alive five years after treatment, making neoplasia the second leading cause of death in the United States. One of the reasons for this poor long term survival rate is that many patients develop multidrug resistant neoplasias. After several cycles of chemotherapy, some tumor cells become resistant not only to the agent used in the chemotherapy, but also to compounds with different structures and mechanisms of action. It is believed that the ATP binding cassette superfamily of transporter proteins acts as an energy-dependent drug efflux pump and alterations in these transporter proteins are associated with the development of multi-drug resistant neoplasias. The activity of this family of proteins prevents the intracellular accumulation of a broad range of cytotoxic drugs.

### SUMMARY OF THE INVENTION

One aspect of the invention provides a compound of Formula (I):

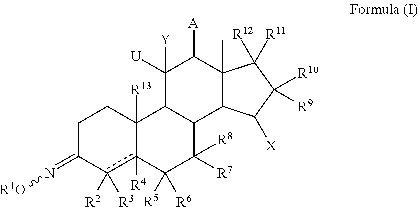

wherein:

[symbol] represents a single bond or double bond in the ring structure of Formula (I);

A and X, for each occurrence, independently is H or halogen;

One of U and Y is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸;

or U and Y together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;

R¹ is aminoalkyl, alkyl, or a 4- to 6-membered heterocyclic ring, wherein said aminoalkyl is optionally substituted by one or more alkyl or acetyl; said alkyl, for each occurrence, independently is optionally substituted by guanidinyl, heteroaryl, or a 4- to 6-membered heterocyclic ring; and each 4- to 6-membered heterocyclic ring independently is optionally substituted by one or more alkyl;

One of R² and R³ is H, halogen, optionally substituted (C₁-C₆)alkyl, OH, or absent, and the other is H, halogen, optionally substituted aryl, optionally substituted heteroaryl, (C₁-C₆)alkyl, OH, or absent;

Or R² and R³ together with the carbon to which they are attached form cycloalkyl;

R⁴ is H, —OH, or absent;

One of R⁵ and R⁶ is H, —OH, or halogen, and the other is H; —OR¹⁴; alkyl optionally substituted with hydroxyl or alkoxy; —C(O)—NH₂; —C(O)—O-alkyl; —NHR¹⁵; or alkynyl;

or R⁵ and R⁶ together with the carbon to which they are attached form C═O, C═C(R¹⁶)₂, or C═N—OR²⁰;

One of R⁷ and R⁸ is H, or halogen, and the other is H; —OR¹⁴; alkyl optionally substituted with hydroxyl or alkoxy; —C(O)—NH₂; —C(O)—O-alkyl; —NHR¹⁵; alkynyl; or R⁷ and R⁸ together with the carbon to which they are attached form C═O, C═C(R¹⁶)₂, C═N—OR²⁰, or a cyclopropyl ring;

One of R⁹ and R¹⁰ is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸; or R⁹ and R¹⁰ together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;

One of R¹¹ and R¹² is H, and the other is —OH; alkoxy optionally substituted with a hydroxyl or an amino group; optionally substituted heterocyclyl; optionally substituted heteroaryl; or —OC(O)R¹⁹;

or R¹¹ and R¹² together with the carbon atom to which they are attached form C═O;

R¹³ is H, —CH₂OR¹⁷, —CHO, —COOR¹⁷, —COR¹⁷, —C≡C—R¹⁷, —C(O)NR¹⁷R¹⁸, —(C₁-C₆)alkyl, or a heteroaryl, wherein said (C₁-C₆)alkyl is further optionally substituted by one or more substituents selected from the group of (C₁-C₄)alkyl, hydroxyl, halogen, alkoxy, aryl, —C(O)—NR¹⁷R¹⁸, —OC(O)-alkyl, —C(O)—O-alkyl, cycloalkyl, heteroaryl, and —NR¹⁷R¹⁸; and said heteroaryl is further optionally substituted by (C₁-C₄)alkyl, halogen, hydroxyl, alkoxy, arylalkyl, or cycloalkyl(C₁-C₄)alkyl;

R¹⁴ is H, alkyl, or —NO₂;

R¹⁵ is H or formyl;

R¹⁶, for each occurrence, independently is H or halogen;

R¹⁷ and R¹⁸, for each occurrence, independently is H, alkyl, alkenyl, alkynyl, cycloalkyl, aryl, aminocarbonyl, alkoxycarbonyl, amino, or halogen; wherein R¹⁷ and R¹⁸ independently are further optionally substituted with one or more moieties selected from the group of alkyl, alkenyl, alkynyl, amino, alkoxy, and cycloalkyl,

R¹⁹ is optionally substituted alkyl, or optionally substituted aryl;

R²⁰ is amino(C₁-C₆)alkyl, (C₁-C₆)alkyl, or a 4- to 6-membered heterocyclic ring, wherein said amino(C₁-C₆)alkyl is optionally substituted by one or more (C₁-C₆)alkyl or acetyl; each heterocyclic ring independently is optionally substituted by one or more (C₁-C₆)alkyl; and each (C₁-C₆)alkyl, independently, is optionally substituted by guanidinyl, heteroaryl, or a 4- to 6-membered heterocyclic ring; or a tautomer, stereoisomer, Z and/or E isomer, optical isomer, N-oxide, hydrate, solvate, polymorph, pharmaceutically acceptable ester, amide, salt, prodrug, and isotopic derivative thereof;

provided that

when [symbol] represents a single bond, R¹³ is methyl, and R⁹ and R¹⁰ are both hydrogen, one of R² and R³ must be an optionally substituted aryl or optionally substituted heteroaryl; and

when [symbol] represents a double bond and R¹³ is methyl, R⁹ and R¹⁰ cannot be both hydrogen at the same time.

In one embodiment, A and X are both H. In another embodiment, both of U and Y are H.

One embodiment provides a compound of Formula (I), in which R¹¹ and R¹² together with the carbon atom to which they are attached form C═O.

An embodiment provides that R¹ in Formula (I) is amino(C₁-C₆)alkyl. Another embodiment provides that R¹ is a 4- to 6-membered heterocyclic ring. Still another embodiment provides that R¹ is (C₁-C₆)alkyl substituted by a 4- to 6-membered heterocyclic ring Examples of R¹ include, but are not limited to, aminoethyl, pyrrolidinyl, or piperidinylethyl.

In a certain embodiment, the compound of Formula (I) has [symbol] being a single bond. In an embodiment, R¹³ is unsubstituted (C₁-C₆)alkyl. In one instance, R⁴ is H. In another instance, R⁴ is —OH.

In one embodiment, the compounds of Formula (I) have one of R⁹ and R¹⁰ being H, and the other being halogen. Certain examples provide that one of R⁹ and R¹⁰ is H, and the other is F.

In an embodiment, R⁷ and R⁸ are both H. In a separate embodiment, both of R² and R³ are H.

One embodiment provides that R⁵ and R⁶ are both H at the same time. Another embodiment provides that R⁵ and R⁶ together with the carbon to which they are attached form C═O. In another embodiment, R⁵ and R⁶ together with the carbon to which they are attached form C═C(R¹⁶)₂, whereas R¹⁶ for each occurrence is the same, and can be H or F. In still another embodiment, R⁵ and R⁶ together with the carbon to which they are attached form C═N—OR²⁰. And R²⁰ can be, for example, (C₁-C₆)alkyl.

Certain compounds of Formula (I) include compounds as follows:
16-Fluoro-10,13-dimethyl-tetradecahydro-cyclopenta[a]phenanthrene-3,17-dione 3-[O-(2-amino-ethyl)-oxime]:

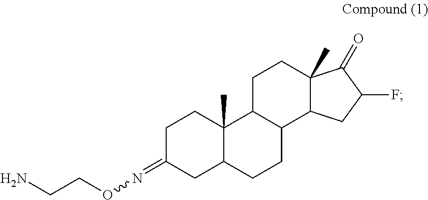 [SMILES: C[C@]12CCC(=NOCCN)CC1CCC1C2CC[C@]2(C)C(=O)C(F)CC12]

16-Fluoro-10,13-dimethyl-tetradecahydro-cyclopenta[a]phenanthrene-3,17-dione 3-(O-pyrrolidin-3-yl-oxime):

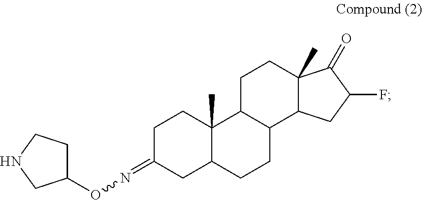 [SMILES: C[C@]12CCC(=NOC3CCNC3)CC1CCC1C2CC[C@]2(C)C(=O)C(F)CC12]

16-Fluoro-10,13-dimethyl-tetradecahydro-cyclopenta[a]phenanthrene-3,6,17-trione 3-[O-(2-amino-ethyl)-oxime]:

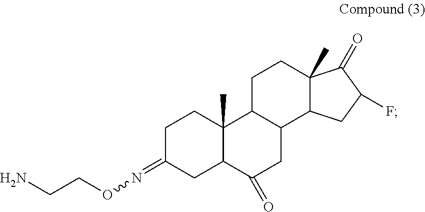 [SMILES: C[C@]12CCC(=NOCCN)CC1C(=O)CC1C2CC[C@]2(C)C(=O)C(F)CC12]

16-Fluoro-10,13-dimethyl-tetradecahydro-cyclopenta[a]phenanthrene-3,6,17-trione 3-(O-pyrrolidin-3-yl-oxime):

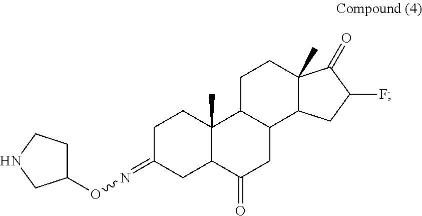 [SMILES: C[C@]12CCC(=NOC3CCNC3)CC1C(=O)CC1C2CC[C@]2(C)C(=O)C(F)CC12]

16-Fluoro-10,13-dimethyl-6-methylene-tetradecahydro-cyclopenta[a]phenanthrene-3,17-dione 3-[O-(2-amino-ethyl)-oxime]:

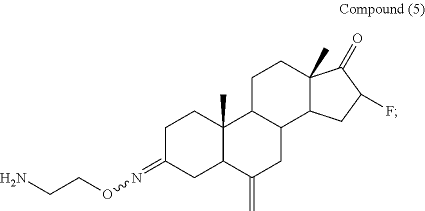 [SMILES: C=C1CC2C(CC[C@]3(C)C(=O)C(F)CC23)[C@@]2(C)CCC(=NOCCN)CC12]

16-Fluoro-10,13-dimethyl-6-methylene-tetradecahydro-cyclopenta[a]phenanthrene-3,17-dione 3-(O-pyrrolidin-3-yl-oxime):

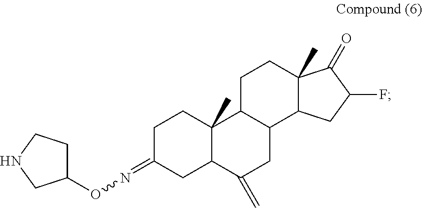 [SMILES: C=C1CC2C(CC[C@]3(C)C(=O)C(F)CC23)[C@@]2(C)CCC(=NOC3CCNC3)CC12]

16-Fluoro-10,13-dimethyl-tetradecahydro-cyclopenta[a]phenanthrene-3,6,17-trione 3-[O-(2-amino-ethyl)-oxime]6-(O-methyl-oxime):

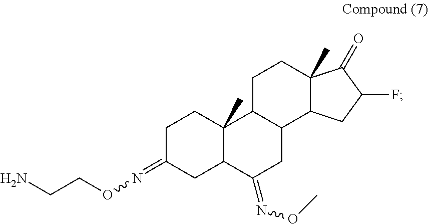 [SMILES: CON=C1CC2C(CC[C@]3(C)C(=O)C(F)CC23)[C@@]2(C)CCC(=NOCCN)CC12]

16-Fluoro-10,13-dimethyl-tetradecahydro-cyclopenta[a]phenanthrene-3,6,17-trione 6-(O-methyl-oxime) 3-(O-pyrrolidin-3-yl-oxime):

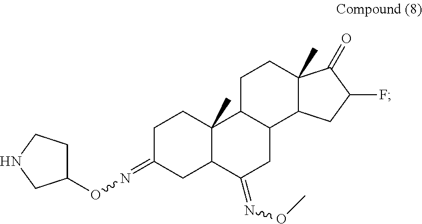 [SMILES: CON=C1CC2C(CC[C@]3(C)C(=O)C(F)CC23)[C@@]2(C)CCC(=NOC3CCNC3)CC12]

16-Fluoro-5-hydroxy-10,13-dimethyl-6-methylene-tetradecahydro-cyclopenta[a]phenanthrene-3,17-dione 3-(O-pyrrolidin-3-yl-oxime):

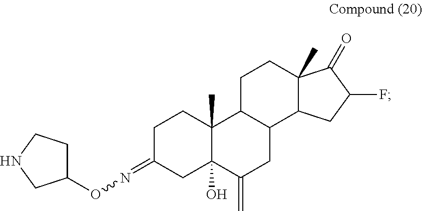 [SMILES: C=C1CC2C3CC(F)C(=O)[C@@]3(C)CCC2[C@@]2(C)CCC(=NOC3CCNC3)C[C@]12O]

16-Fluoro-10,13-dimethyl-tetradecahydro-cyclopenta[a]phenanthrene-3,17-dione 3-[O-(2-piperidin-1-yl-ethyl)-oxime]:

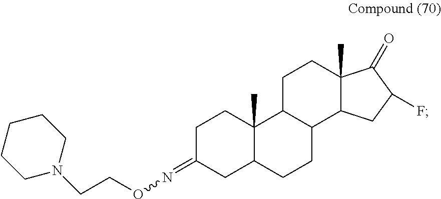 [SMILES: C[C@]12CCC(=NOCCN3CCCCC3)CC1CCC1C2CC[C@]2(C)C(=O)C(F)CC12]

16-Fluoro-10,13-dimethyl-6-methylene-tetradecahydro-cyclopenta[a]phenanthrene-3,17-dione 3-[O-(2-piperidin-1-yl-ethyl)-oxime]:

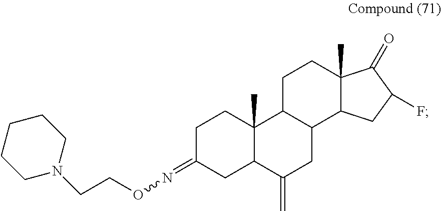 [SMILES: C=C1CC2C(CC[C@]3(C)C(=O)C(F)CC23)[C@@]2(C)CCC(=NOCCN3CCCCC3)CC12]

and
16-Fluoro-10,13-dimethyl-tetradecahydro-cyclopenta[a]phenanthrene-3,6,17-trione 6-(O-methyl-oxime) 3-[O-(2-piperidin-1-yl-ethyl)-oxime]:

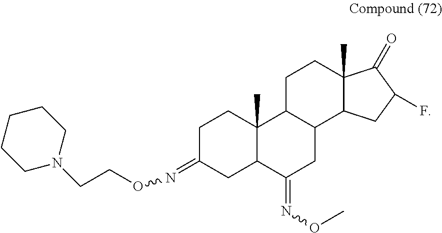 [SMILES: CON=C1CC2C(CC[C@]3(C)C(=O)C(F)CC23)[C@@]2(C)CCC(=NOCCN3CCCCC3)CC12]

In another embodiment of the invention, the compound of Formula (I) has one of R² and R³ being H, the other being an optionally substituted (C₁-C₆)alkyl or an optionally substituted aryl. One of the examples is that one of R² and R³ is H, and the other is phenyl. Another example is that one of R² and R³ is H, and the other is ethyl.

One embodiment provides that R⁵ and R⁶ together with the carbon to which they are attached form C═O. Another embodiment provides that R⁵ and R⁶ together with the carbon to which they are attached form C═C(R¹⁶)₂, whereas R¹⁶ for each occurrence is the same and can be H or F. Still another embodiment provides that R⁵ and R⁶ together with the carbon to which they are attached form C═N—OR²⁰. And R²⁰ can be, for example, (C₁-C₆)alkyl.

Compounds of Formula (I) also include, for example, compounds as follows:
4-Ethyl-16-fluoro-10,13-dimethyl-tetradecahydro-cyclopenta[a]phenanthrene-3,17-dione 3-(O-pyrrolidin-3-yl-oxime):

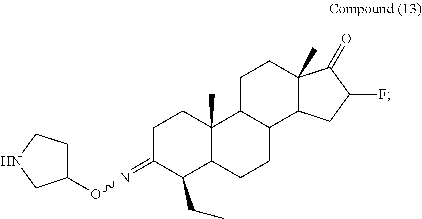 [SMILES: CC[C@H]1C(=NOC2CCNC2)CC[C@]2(C)C3CC[C@]4(C)C(=O)C(F)CC4C3CCC12]

16-Fluoro-10,13-dimethyl-4-phenyl-tetradecahydro-cyclopenta[a]phenanthrene-3,17-dione 3-(O-pyrrolidin-3-yl-oxime):

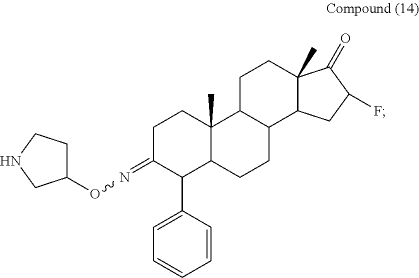 [SMILES: C[C@]12CCC(=NOC3CCNC3)C(c3ccccc3)C1CCC1C2CC[C@]2(C)C(=O)C(F)CC12]

4-Ethyl-16-fluoro-10,13-dimethyl-tetradecahydro-cyclopenta[a]phenanthrene-3,6,17-trione 3-(O-pyrrolidin-3-yl-oxime):

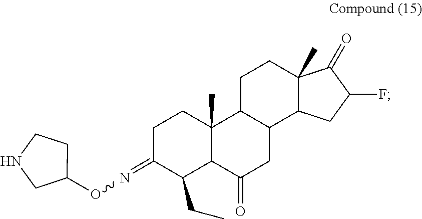 [SMILES: CC[C@H]1C(=NOC2CCNC2)CC[C@]2(C)C3CC[C@]4(C)C(=O)C(F)CC4C3CC(=O)C12]

4-Ethyl-16-fluoro-10,13-dimethyl-6-methylene-tetradecahydro-cyclopenta[a]phenanthrene-3,17-dione 3-(O-pyrrolidin-3-yl-oxime):

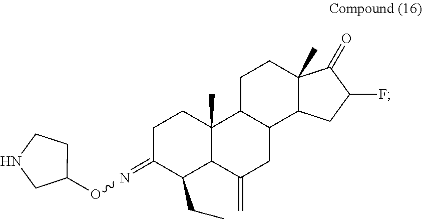 [SMILES: C=C1CC2C(CC[C@]3(C)C(=O)C(F)CC23)[C@@]2(C)CCC(=NOC3CCNC3)[C@H](CC)C12]

4-Ethyl-16-fluoro-10,13-dimethyl-tetradecahydro-cyclopenta[a]phenanthrene-3,6,17-trione 6-(O-methyl-oxime) 3-(O-pyrrolidin-3-yl-oxime):

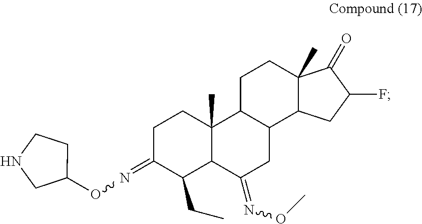 [SMILES: CC[C@H]1C(=NOC2CCNC2)CC[C@]2(C)C3CC[C@]4(C)C(=O)C(F)CC4C3CC(=NOC)C12]

16-Fluoro-10,13-dimethyl-6-methylene-4-phenyl-tetradecahydro-cyclopenta[a]phenanthrene-3,17-dione 3-(O-pyrrolidin-3-yl-oxime):

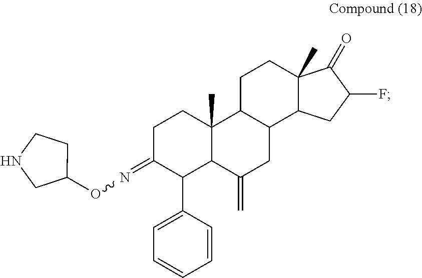 [SMILES: C=C1CC2C(CC[C@]3(C)C(=O)C(F)CC23)[C@@]2(C)CCC(=NOC3CCNC3)C(c3ccccc3)C12]

and
16-Fluoro-10,13-dimethyl-4-phenyl-tetradecahydro-cyclopenta[a]phenanthrene-3,6,17-trione 6-(O-methyl-oxime) 3-(O-pyrrolidin-3-yl-oxime):

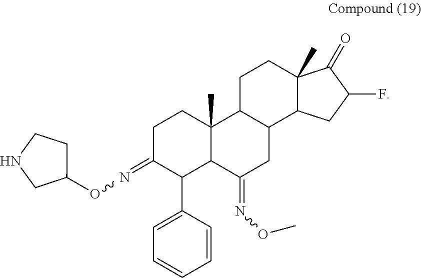 [SMILES: CON=C1CC2C(CC[C@]3(C)C(=O)C(F)CC23)[C@@]2(C)CCC(=NOC3CCNC3)C(c3ccccc3)C12]

Another embodiment provides a compound of Formula (I) with R⁷ and R⁸ together with the carbon to which they are attached forming C═O. In a separate embodiment, R⁷ and R⁸ together with the carbon to which they are attached form C═C(R¹⁶)₂. In certain embodiments, R¹⁶ is the same at each occurrence. R¹⁶ can be, for example, H or F.

In a certain embodiment, R² and R³ are both H.

In one embodiment, R⁵ and R⁶ are both H. In another embodiment, one of R⁵ and R⁶ is H, and the other is hydroxymethyl.

Exemplified compounds include, but are not limited to, the following compounds:
16-Fluoro-10,13-dimethyl-7-methylene-tetradecahydro-cyclopenta[a]phenanthrene-3,17-dione 3-(O-pyrrolidin-3-yl-oxime):

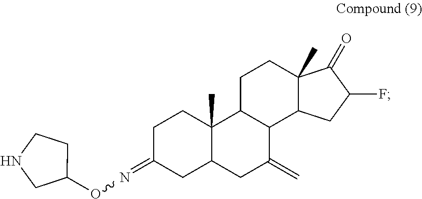 [SMILES: C=C1CC2CC(=NOC3CCNC3)CC[C@]2(C)C2CC[C@]3(C)C(=O)C(F)CC3C12]

7-Difluoromethylene-16-fluoro-10,13-dimethyl-tetradecahydro-cyclopenta[a]phenanthrene-3,17-dione 3-(O-pyrrolidin-3-yl-oxime):

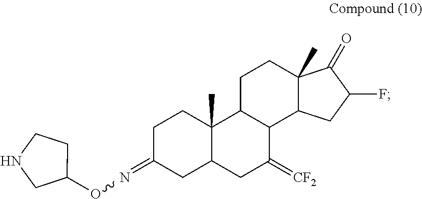

16-Fluoro-6-hydroxymethyl-10,13-dimethyl-dodecahydro-cyclopenta[a]phenanthrene-3,7,17-trione 3-[O-(2-amino-ethyl)-oxime]:

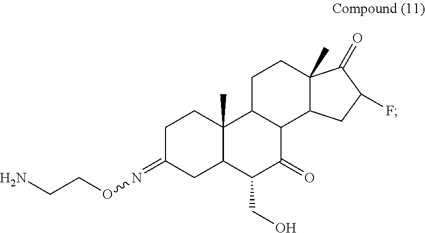 [SMILES: C[C@]12CCC(=NOCCN)CC1[C@@H](CO)C(=O)C1C2CC[C@]2(C)C(=O)C(F)CC12]

and
16-Fluoro-6-hydroxymethyl-10,13-dimethyl-dodecahydro-cyclopenta[a]phenanthrene-3,7,17-trione 3-(O-pyrrolidin-3-yl-oxime):

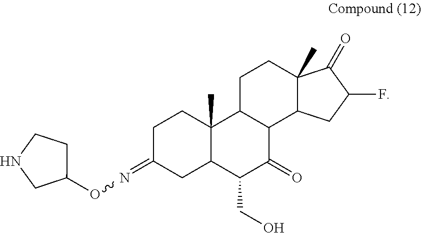 [SMILES: C[C@]12CCC(=NOC3CCNC3)CC1[C@@H](CO)C(=O)C1C2CC[C@]2(C)C(=O)C(F)CC12]

Other embodiments provide compounds of Formula (I) with one of R⁹ and R¹⁰ being H, and the other being F, in which one of R⁷ and R⁸ is H, and the other is —OH. In a certain embodiment, R² and R³ are both H. One embodiment provides that R⁵ and R⁶ are both H. Another embodiment provides that one of R⁵ and R⁶ is H, and the other is hydroxymethyl.

Exemplified compounds include the following compound:
16-Fluoro-7-hydroxy-6-hydroxymethyl-10,13-dimethyl-tetradecahydro-cyclopenta[a]phenanthrene-3,17-dione 3-(O-pyrrolidin-3-yl-oxime):

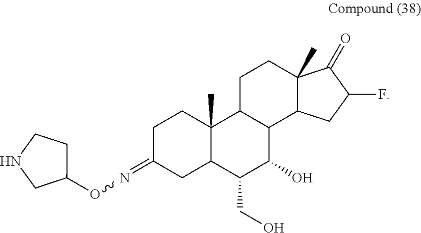 [SMILES: C[C@]12CCC(=NOC3CCNC3)CC1[C@@H](CO)[C@@H](O)C1C2CC[C@]2(C)C(=O)C(F)CC12]

In still another embodiment, the compounds of Formula (I), in which one of R⁹ and R¹⁰ is H and the other is F, have R⁷ and R⁸ together with the carbon to which they are attached to form C═N—OR²⁰.

In a separate embodiment, the compounds have both of R⁹ and R¹⁰ being H. One of R² and R³ may be H, while the other may be an optionally substituted aryl.

In one embodiment, both of R⁷ and R⁸ are H. In another embodiment, R⁷ and R⁸ together with the carbon to which they are attached form C═N—OR²⁰.

In one embodiment, R⁵ and R⁶ together with the carbon to which they are attached form C═C(R¹⁶)₂. And R¹⁶, for each occurrence, may be the same. Examples of R¹⁶ include H and F. In another embodiment, both of R⁵ and R⁶ are H. In still another embodiment, R⁵ and R⁶ together with the carbon to which they are attached form C═N—OR²⁰. R²⁰ can be, for example, (C₁-C₆)alkyl.

Exemplified compounds further include the following compounds:
10,13-Dimethyl-4-phenyl-tetradecahydro-cyclopenta[a]phenanthrene-3,17-dione 3-(O-pyrrolidin-3-yl-oxime):

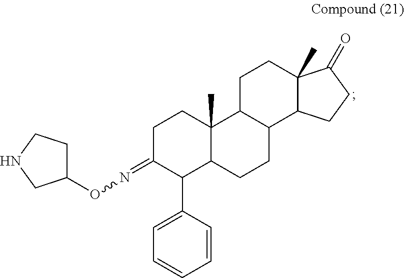 [SMILES: C[C@]12CCC(=NOC3CCNC3)C(c3ccccc3)C1CCC1C2CC[C@]2(C)C(=O)CCC12]

10,13-Dimethyl-6-methylene-4-phenyl-tetradecahydro-cyclopenta[a]phenanthrene-3,17-dione 3-(O-pyrrolidin-3-yl-oxime):

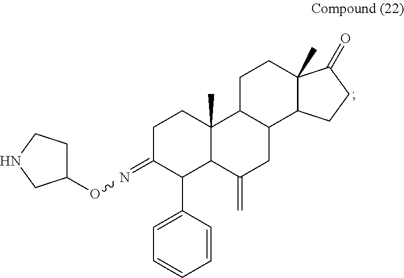 [SMILES: C=C1CC2C(CC[C@]3(C)C(=O)CCC23)[C@@]2(C)CCC(=NOC3CCNC3)C(c3ccccc3)C12]

and
10,13-Dimethyl-4-phenyl-tetradecahydro-cyclopenta[a]phenanthrene-3,6,17-trione 6-(O-methyl-oxime) 3-(O-pyrrolidin-3-yl-oxime):

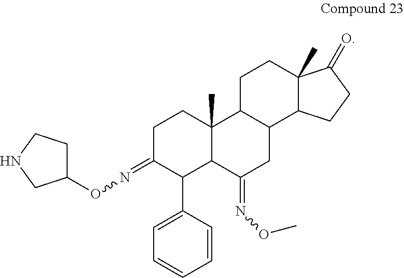 [SMILES: CON=C1CC2C(CC[C@]3(C)C(=O)CCC23)[C@@]2(C)CCC(=NOC3CCNC3)C(c3ccccc3)C12]

In another separate embodiment of the compounds, R¹³ is a hydroxyl-substituted (C₁-C₆)alkyl moiety.

In one embodiment, R⁴ is H. Another embodiment provides that R⁴ is —OH. In a certain embodiment, both of R⁹ and R¹⁰ are H. In another embodiment, one of R⁹ and R¹⁰ is H, and the other is F.

In certain instances, R⁷ and R⁸ are both H. One embodiment provides that both R² and R³ are H. Other embodiments provide that one of R² and R³ is H, and the other is an optionally substituted aryl or an optionally substituted (C₁-C₆)alkyl. R⁵ and R⁶ can be both H, or R⁵ and R⁶ together with the carbon to which they are attached form C═O or C═C(R¹⁶)₂. In certain embodiments, R¹⁶ for each occurrence is the same, and can be H or F. Still further, R⁵ and R⁶ together with the carbon to which they are attached may form C═N—OR²⁰. R²⁰ can be, for example, (C₁-C₆)alkyl.

In other instances where R¹³ is a hydroxyl-substituted (C₁-C₆)alkyl moiety, R⁷ and R⁸ together with the carbon to which they are attached may form C═O. Still further, R⁷ and R⁸ together with the carbon to which they are attached may form C═C(R¹⁶)₂. In certain embodiments, R¹⁶ is the same on each occurrence, and can be H or F.

One embodiment provides that both R² and R³ are H.

In one embodiment, the compounds have both of R⁵ and R⁶ being H. In another embodiment, one of R⁵ and R⁶ is H, and the other is (C₁-C₆)alkyl substituted by hydroxyl.

Exemplified compounds of the invention further include compounds as follows:
10-Hydroxymethyl-13-methyl-tetradecahydro-cyclopenta[a]phenanthrene-3,17-dione 3-[O-(2-amino-ethyl)-oxime]:

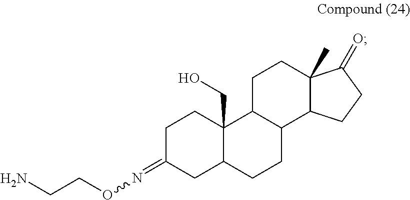 [SMILES: C[C@]12CCC3C(CCC4CC(=NOCCN)CC[C@@]43CO)C1CCC2=O]

10-Hydroxymethyl-13-methyl-tetradecahydro-cyclopenta[a]phenanthrene-3,17-dione 3-(O-pyrrolidin-3-yl-oxime):

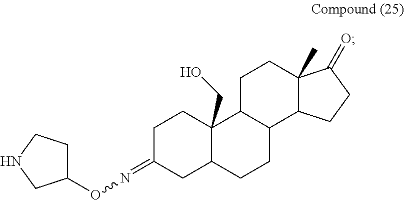 [SMILES: C[C@]12CCC3C(CCC4CC(=NOC5CCNC5)CC[C@@]43CO)C1CCC2=O]

4-Ethyl-10-hydroxymethyl-13-methyl-tetradecahydro-cyclopenta[a]phenanthrene-3,17-dione 3-(O-pyrrolidin-3-yl-oxime):

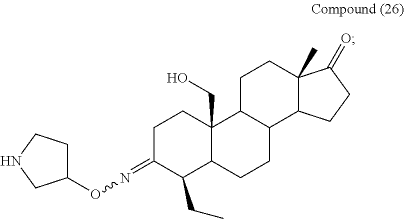 [SMILES: CC[C@H]1C(=NOC2CCNC2)CC[C@]2(CO)C3CC[C@]4(C)C(=O)CCC4C3CCC12]

10-Hydroxymethyl-13-methyl-4-phenyl-tetradecahydro-cyclopenta[a]phenanthrene-3,17-dione 3-(O-pyrrolidin-3-yl-oxime):

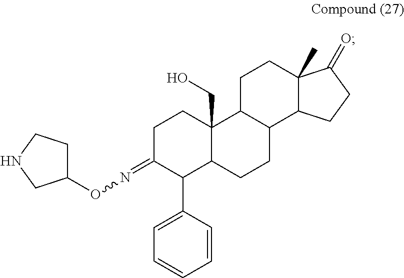 [SMILES: C[C@]12CCC3C(CCC4C(c5ccccc5)C(=NOC5CCNC5)CC[C@@]43CO)C1CCC2=O]

10-Hydroxymethyl-13-methyl-tetradecahydro-cyclopenta[a]phenanthrene-3,6,17-trione 3-(O-pyrrolidin-3-yl-oxime):

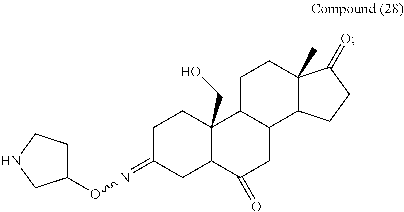 [SMILES: C[C@]12CCC3C(CC(=O)C4CC(=NOC5CCNC5)CC[C@@]43CO)C1CCC2=O]

10-Hydroxymethyl-13-methyl-6-methylene-tetradecahydro-cyclopenta[a]phenanthrene-3,17-dione 3-(O-pyrrolidin-3-yl-oxime):

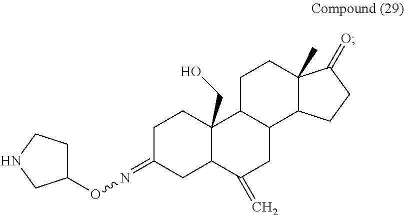 [SMILES: C=C1CC2C(CC[C@]3(C)C(=O)CCC23)[C@@]2(CO)CCC(=NOC3CCNC3)CC12]

10-Hydroxymethyl-13-methyl-tetradecahydro-cyclopenta[a]phenanthrene-3,6,17-trione 6-(O-methyl-oxime) 3-(O-pyrrolidin-3-yl-oxime):

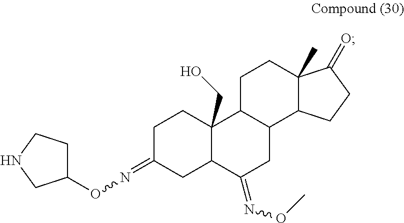 [SMILES: CON=C1CC2C(CC[C@]3(C)C(=O)CCC23)[C@@]2(CO)CCC(=NOC3CCNC3)CC12]

4-Ethyl-10-hydroxymethyl-13-methyl-tetradecahydro-cyclopenta[a]phenanthrene-3,6,17-trione 3-(O-pyrrolidin-3-yl-oxime):

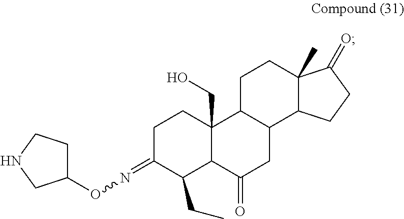 [SMILES: CC[C@H]1C(=NOC2CCNC2)CC[C@]2(CO)C3CC[C@]4(C)C(=O)CCC4C3CC(=O)C12]

4-Ethyl-10-hydroxymethyl-13-methyl-6-methylene-tetradecahydro-cyclopenta[a]phenanthrene-3,17-dione 3-(O-pyrrolidin-3-yl-oxime):

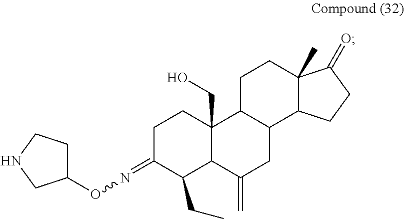 [SMILES: C=C1CC2C(CC[C@]3(C)C(=O)CCC23)[C@@]2(CO)CCC(=NOC3CCNC3)[C@H](CC)C12]

4-Ethyl-10-hydroxymethyl-13-methyl-tetradecahydro-cyclopenta[a]phenanthrene-3,6,17-trione 6-(O-methyl-oxime) 3-(O-pyrrolidin-3-yl-oxime):

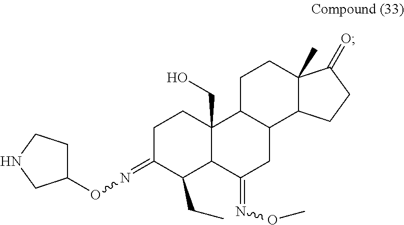 [SMILES: CC[C@H]1C(=NOC2CCNC2)CC[C@]2(CO)C3CC[C@]4(C)C(=O)CCC4C3CC(=NOC)C12]

5-Hydroxy-10-hydroxymethyl-13-methyl-6-methylene-tetradecahydro-cyclopenta[a]phenanthrene-3,17-dione 3-(O-pyrrolidin-3-yl-oxime):

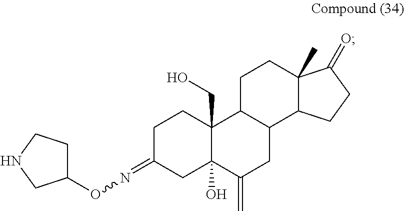 [SMILES: C=C1CC2C(CC[C@]3(C)C(=O)CCC23)[C@@]2(CO)CCC(=NOC3CCNC3)C[C@]12O]

10-Hydroxymethyl-13-methyl-7-methylene-tetradecahydro-cyclopenta[a]phenanthrene-3,17-dione 3-(O-pyrrolidin-3-yl-oxime):

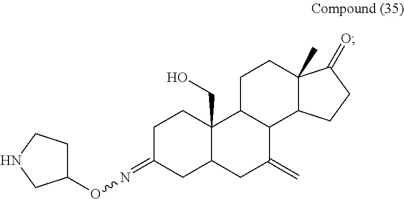 [SMILES: C=C1CC2CC(=NOC3CCNC3)CC[C@]2(CO)C2CC[C@]3(C)C(=O)CCC3C12]

7-Difluoromethylene-10-hydroxymethyl-13-methyl-tetradecahydro-cyclopenta[a]phenanthrene-3,17-dione 3-(O-pyrrolidin-3-yl-oxime):

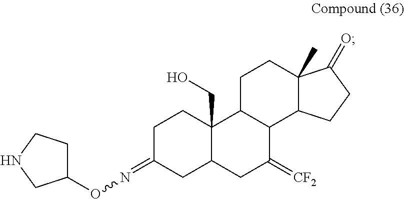

and
6,10-Bis-hydroxymethyl-13-methyl-dodecahydro-cyclopenta[a]phenanthrene-3,7,17-trione 3-(O-pyrrolidin-3-yl-oxime):

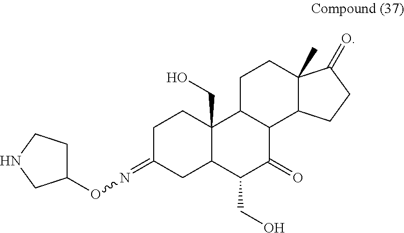 [SMILES: C[C@]12CCC3C(C(=O)[C@H](CO)C4CC(=NOC5CCNC5)CC[C@]34CO)C1CCC2=O]

In still other embodiments where R¹³ is a hydroxyl-substituted (C₁-C₆)alkyl, one of R⁷ and R⁸ is H, and the other is —OH. Certain embodiments provide that both R² and R³ are H. In one embodiment, one of R⁵ and R⁶ is H, and the other is (C₁-C₆)alkyl substituted by hydroxyl or (C₁-C₄)alkoxy.

Compounds of the invention also include, for example, the following compound:
7-Hydroxy-6,10-bis-hydroxymethyl-13-methyl-tetradecahydro-cyclopenta[a]phenanthrene-3,17-dione 3-(O-pyrrolidin-3-yl-oxime):

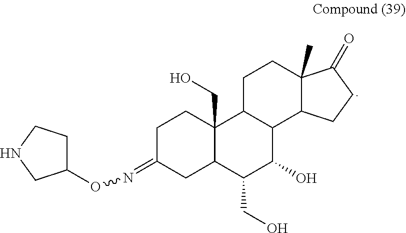 [SMILES: C[C@]12CCC3C(C1CCC2=O)[C@H](O)[C@H](CO)C1CC(=NOC2CCNC2)CC[C@]31CO]

In a separate embodiment of the invention, R¹³ is (C₁-C₆)alkyl that is substituted by —OC(O)—(C₁-C₆)alkyl. R¹³ can be, for example, —CH₂OC(O)CH₃.

Certain embodiments provide that both of R⁹ and R¹⁰ are H, or one of R⁹ and R¹⁰ is H, and the other is F.

One embodiment provides that R⁷ and R⁸ are both H. Another embodiment provides that one of R⁷ and R⁸ is H, and the other is —OH. Still another embodiment provides that R⁷ and R⁸ together with the carbon to which they are attached form C═O.

R² and R³ can be, for example, both H. Or, one of R² and R³ is H, and the other is an optionally substituted aryl or an optionally substituted (C₁-C₆)alkyl.

In one embodiment, R⁵ and R⁶ are both H. In another embodiment, one of R⁵ and R⁶ is H, the other is —CH₂OH. In another embodiment, R⁵ and R⁶ together with the carbon to which they are attached form C═CH₂. Another embodiment provides that R⁵ and R⁶ together with the carbon to which they are attached form C═N—OR²⁰. R²⁰ can be, for example, (C₁-C₆)alkyl.

The invention further provides the following exemplified compounds:
Acetic acid 13-methyl-6-methylene-17-oxo-3-(pyrrolidin-3-yloxyimino)-hexadecahydro-cyclopenta[a]phenanthren-10-ylmethyl ester:

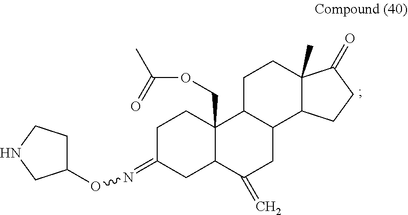 [SMILES: C=C1CC2C(CC[C@]3(C)C(=O)CCC23)[C@@]2(COC(C)=O)CCC(=NOC3CCNC3)CC12]

Acetic acid 6-methoxyimino-13-methyl-17-oxo-3-(pyrrolidin-3-yloxyimino)-hexadecahydro-cyclopenta[a]phenanthren-10-ylmethyl ester:

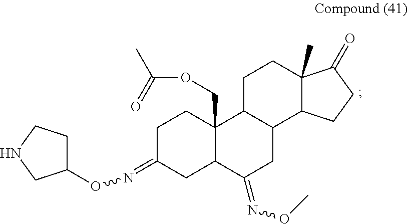 [SMILES: CON=C1CC2C(CC[C@]3(C)C(=O)CCC23)[C@@]2(COC(C)=O)CCC(=NOC3CCNC3)CC12]

Acetic acid 4-ethyl-13-methyl-6-methylene-17-oxo-3-(pyrrolidin-3-yloxyimino)-hexadecahydro-cyclopenta[a]phenanthren-10-ylmethyl ester:

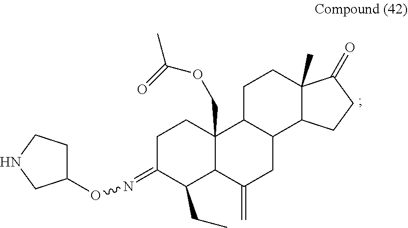 [SMILES: C=C1CC2C(CC[C@]3(C)C(=O)CCC23)[C@@]2(COC(C)=O)CCC(=NOC3CCNC3)[C@H](CC)C12]

Acetic acid 4-ethyl-6-methoxyimino-13-methyl-17-oxo-3-(pyrrolidin-3-yloxyimino)-hexadecahydro-cyclopenta[a]phenanthren-10-ylmethyl ester:

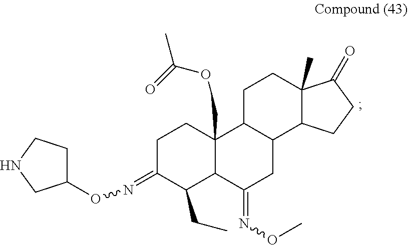 [SMILES: CC[C@H]1C(=NOC2CCNC2)CC[C@]2(COC(C)=O)C3CC[C@]4(C)C(=O)CCC4C3CC(=NOC)C12]

Acetic acid 13-methyl-6-methylene-17-oxo-4-phenyl-3-(pyrrolidin-3-yloxyimino)-hexadecahydro-cyclopenta[a]phenanthren-10-ylmethyl ester:

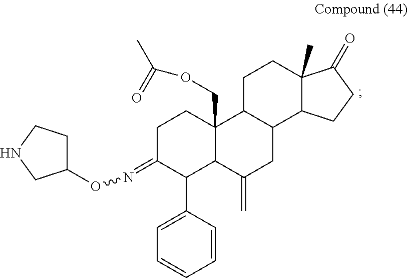 [SMILES: C=C1CC2C(CC[C@]3(C)C(=O)CCC23)[C@@]2(COC(C)=O)CCC(=NOC3CCNC3)C(c3ccccc3)C12]

Acetic acid 6-methoxyimino-13-methyl-17-oxo-4-phenyl-3-(pyrrolidin-3-yloxyimino)-hexadecahydro-cyclopenta[a]phenanthren-10-ylmethyl ester:

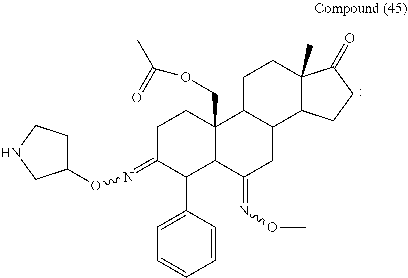 [SMILES: CON=C1CC2C(CC[C@]3(C)C(=O)CCC23)[C@@]2(COC(C)=O)CCC(=NOC3CCNC3)C(c3ccccc3)C12]

Acetic acid 6-hydroxymethyl-13-methyl-7,17-dioxo-3-(pyrrolidin-3-yloxyimino)-hexadecahydro-cyclopenta[a]phenanthren-10-ylmethyl ester:

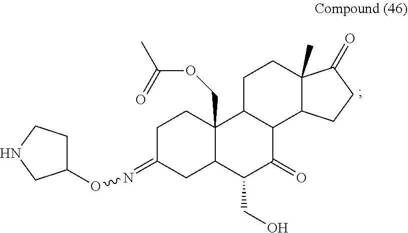 [SMILES: CC(=O)OC[C@]12CCC(=NOC3CCNC3)CC1[C@@H](CO)C(=O)C1C2CC[C@]2(C)C(=O)CCC12]

Acetic acid 7-hydroxy-6-hydroxymethyl-13-methyl-17-oxo-3-(pyrrolidin-3-yloxyimino)-hexadecahydro-cyclopenta[a]phenanthren-10-ylmethyl ester:

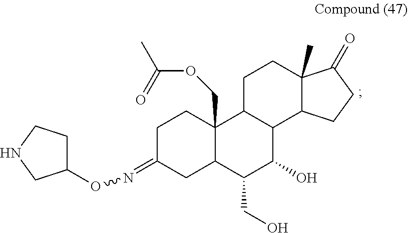 [SMILES: CC(=O)OC[C@]12CCC(=NOC3CCNC3)CC1[C@@H](CO)[C@@H](O)C1C2CC[C@]2(C)C(=O)CCC12]

and
Acetic acid 13-methyl-17-oxo-3-(pyrrolidin-3-yloxyimino)-hexadecahydro-cyclopenta[a]phenanthren-10-ylmethyl ester:

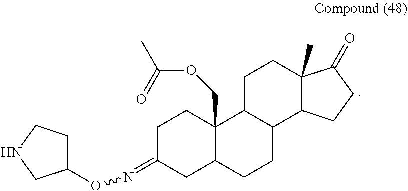 [SMILES: CC(=O)OC[C@]12CCC(=NOC3CCNC3)CC1CCC1C2CC[C@]2(C)C(=O)CCC12]

In a separate embodiment of the invention, [symbol] in the compounds of Formula (I) represents a double bond.

One embodiment provides that R¹³ is an optionally substituted (C₁-C₆)alkyl. For example, R¹³ can be methyl, —CH₂OC(O)CH₃, or —CH₂OH.

In one embodiment, both of R⁹ and R¹⁰ are H. In another embodiment, one of R⁹ and R¹⁰ is H, and the other is halogen (e.g., F).

Certain embodiments provide that R⁷ and R⁸ are both H; or one of R⁷ and R⁸ is H, the other is —OH.

In a certain embodiment, R⁵ and R⁶ are both H. In one embodiment, one of R⁵ and R⁶ is H, the other is methyl or —CH₂OH. In another embodiment, R⁵ and R⁶ together with the carbon to which they are attached form C═O or C═C(R¹⁶)₂. In certain instances, R¹⁶ for each occurrence is the same, and can be H or F. In still another embodiment, R⁵ and R⁶ together with the carbon to which they are attached form C═N—OR²⁰. R²⁰ can be, for example, (C₁-C₆)alkyl.

Exemplified compounds of the invention also include, for example, the following compounds:
16-Fluoro-10,13-dimethyl-1,6,7,8,9,10,11,12,13,14,15,16-dodecahydro-2H-cyclopenta[a]phenanthrene-3,17-dione 3-(O-pyrrolidin-3-yl-oxime):

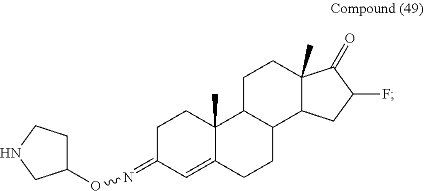 [SMILES: C[C@]12CCC(=NOC3CCNC3)C=C1CCC1C2CC[C@]2(C)C(=O)C(F)CC12]

16-Fluoro-10,13-dimethyl-6-methylene-1,6,7,8,9,10,11,12,13,14,15,16-dodecahydro-2H-cyclopenta[a]phenanthrene-3,17-dione 3-(O-pyrrolidin-3-yl-oxime):

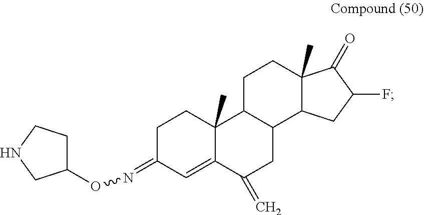 [SMILES: C=C1CC2C(CC[C@]3(C)C(=O)C(F)CC23)[C@@]2(C)CCC(=NOC3CCNC3)C=C12]

6-Ethylimino-16-fluoro-10,13-dimethyl-1,6,7,8,9,10,11,12,13,14,15,16-dodecahydro-2H-cyclopenta[a]phenanthrene-3,17-dione 3-(O-pyrrolidin-3-yl-oxime):

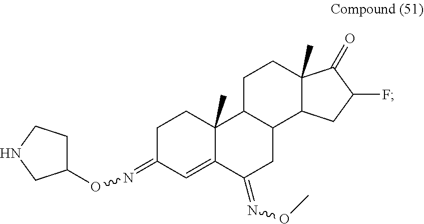 [SMILES: CON=C1CC2C(CC[C@]3(C)C(=O)C(F)CC23)[C@@]2(C)CCC(=NOC3CCNC3)C=C12]

16-Fluoro-7-hydroxy-6-hydroxymethyl-10,13-dimethyl-1,6,7,8,9,10,11,12,13,14,15,16-dodecahydro-2H-cyclopenta[a]phenanthrene-3,17-dione 3-(O-pyrrolidin-3-yl-oxime):

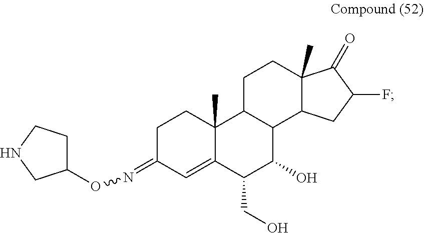 [SMILES: C[C@]12CCC(=NOC3CCNC3)C=C1[C@@H](CO)[C@@H](O)C1C2CC[C@]2(C)C(=O)C(F)CC12]

10-Hydroxymethyl-13-methyl-1,6,7,8,9,10,11,12,13,14,15,16-dodecahydro-2H-cyclopenta[a]phenanthrene-3,17-dione 3-(O-pyrrolidin-3-yl-oxime):

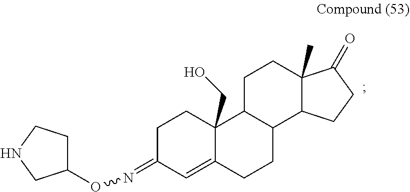 [SMILES: C[C@]12CCC3C(CCC4=CC(=NOC5CCNC5)CC[C@@]43CO)C1CCC2=O]

Acetic acid 13-methyl-17-oxo-3-(pyrrolidin-3-yloxyimino)-1,2,3,6,7,8,9,11,12,13,14,15,16,17-tetradecahydro-cyclopenta[a]phenanthren-10-ylmethyl ester:

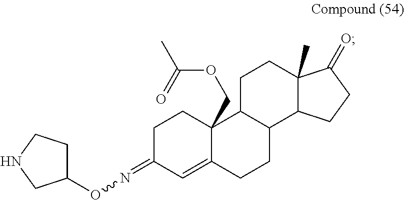 [SMILES: CC(=O)OC[C@]12CCC(=NOC3CCNC3)C=C1CCC1C2CC[C@]2(C)C(=O)CCC12]

10-Hydroxymethyl-13-methyl-6-methylene-1,6,7,8,9,10,11,12,13,14,15,16-dodecahydro-2H-cyclopenta[a]phenanthrene-3,17-dione 3-(O-pyrrolidin-3-yl-oxime):

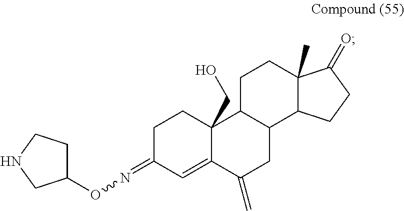 [SMILES: C=C1CC2C(CC[C@]3(C)C(=O)CCC23)[C@@]2(CO)CCC(=NOC3CCNC3)C=C12]

6-Ethylimino-10-hydroxymethyl-13-methyl-1,6,7,8,9,10,11,12,13,14,15,16-dodecahydro-2H-cyclopenta[a]phenanthrene-3,17-dione 3-(O-pyrrolidin-3-yl-oxime):

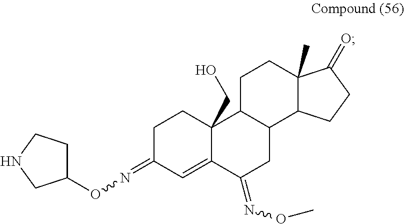 [SMILES: CON=C1CC2C(CC[C@]3(C)C(=O)CCC23)[C@@]2(CO)CCC(=NOC3CCNC3)C=C12]

7-Hydroxy-6,10-bis-hydroxymethyl-13-methyl-1,6,7,8,9,10,11,12,13,14,15,16-dodecahydro-2H-cyclopenta[a]phenanthrene-3,17-dione 3-(O-pyrrolidin-3-yl-oxime):

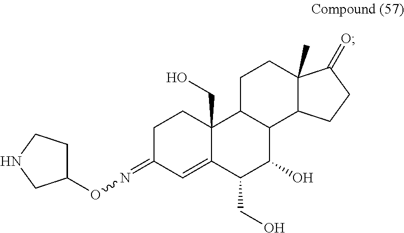 [SMILES: C[C@]12CCC3C(C1CCC2=O)[C@H](O)[C@H](CO)C1=CC(=NOC2CCNC2)CC[C@@]13CO]

16-Fluoro-10,13-dimethyl-6-methylene-1,6,7,8,9,10,11,12,13,14,15,16-dodecahydro-2H-cyclopenta[a]phenanthrene-3,17-dione 3-[O-(2-piperidin-1-yl-ethyl)-oxime]:

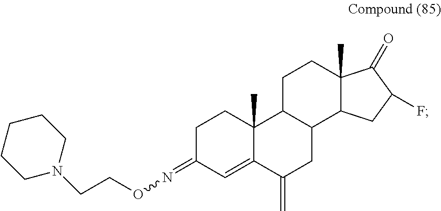 [SMILES: C=C1CC2C(CC[C@]3(C)C(=O)C(F)CC23)[C@@]2(C)CCC(=NOCCN3CCCCC3)C=C12]

and
16-Fluoro-6,10,13-trimethyl-1,6,7,8,9,10,11,12,13,14,15,16-dodecahydro-2H-cyclopenta[a]phenanthrene-3,17-dione 3-(O-pyrrolidin-3-yl-oxime):

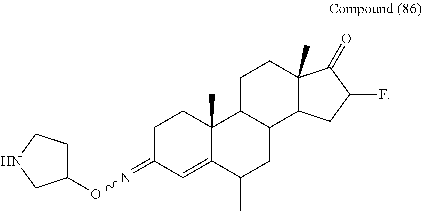 [SMILES: CC1CC2C(CC[C@]3(C)C(=O)C(F)CC23)[C@@]2(C)CCC(=NOC3CCNC3)C=C12]

In a related aspect, the invention provides compounds of Formula (II):

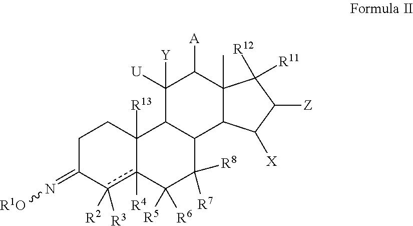

wherein:

[symbol] represents a single or double bond;

A and X, for each occurrence, independently is H or halogen;

One of U and Y is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸;

or U and Y together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;

Z is halogen;

R¹ is amino(C₁-C₆)alkyl, or a 4- to 6-membered heterocyclic ring, or (C₁-C₆)alkyl substituted by a 4- to 6-membered heterocyclic ring (e.g., aminoethyl, pyrrolidinyl, or piperidinylethyl);

One of R² and R³ is H, halogen, optionally substituted (C₁-C₆)alkyl, OH, or absent, and the other is H, halogen, optionally substituted aryl, optionally substituted heteroaryl, (C₁-C₆)alkyl, OH, or absent;

Or R² and R³ together with the carbon to which they are attached form cycloalkyl;

R⁴ is H, —OH, or absent;

One of R⁵ and R⁶ is H, —OH, or halogen, and the other is H; —OR¹⁴; alkyl optionally substituted with hydroxyl or alkoxy; —C(O)NH₂; —C(O)—O-alkyl; —NHR¹⁵; or alkynyl;

or R⁵ and R⁶ together with the carbon to which they are attached form C═O, C═C(R¹⁶)₂, or C═N—OR²⁰;

One of R⁷ and R⁸ is H, or halogen, and the other is H; —OR¹⁴; alkyl optionally substituted with hydroxyl or alkoxy; —C(O)—NH₂; —C(O)—O-alkyl; —NHR¹⁵; alkynyl; or R⁷ and R⁸ together with the carbon to which they are attached form C═O or C═C(R¹⁶)₂;

R¹¹ and R¹² together with the carbon atom to which they are attached form C═O;

R¹³ is an optionally substituted (C₁-C₆)alkyl (e.g., —CH₃, —CH₂OC(O)CH₃ and —CH₂OH);

R¹⁴ is H or alkyl;

R¹⁵ is H or formyl;

R¹⁶, for each occurrence, independently, is H or F;

R¹⁷ and R¹⁸, for each occurrence, independently is H, alkyl, alkenyl, alkynyl, cycloalkyl, aryl, aminocarbonyl, alkoxycarbonyl, amino, or halogen; wherein R¹⁷ and R¹⁸ independently are further optionally substituted with one or more moieties selected from the group of alkyl, alkenyl, alkynyl, amino, alkoxy, and cycloalkyl,

R²⁰ is (C₁-C₆)alkyl.

In certain embodiments, Z is fluorine. Specific embodiments of such compounds of Formula (II) include, but are not limited to, Compound Nos. 1, 3, 5, 7, 49-52, 70-72, 79-81, 85 and 86 20, 49-52, 64-66, 70-72, 79-81, supra.

In another aspect, the invention provides a compound of Formula (Ia):

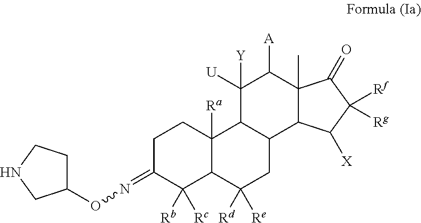

wherein:

A and X, for each occurrence, independently is H or halogen;

One of U and Y is H, and the other is H or halogen; or U and Y together with the carbon to which they are attached form C═O;

R^{a} is H, or (C₁-C₆)alkyl that is further optionally substituted by hydroxyl or —OC(O)—(C₁-C₆)alkyl;

One of R^{b} and R^{c} is H, or halogen, and the other is H, halogen, optionally substituted aryl, optionally substituted heteroaryl, alkyl, or OH;

One of R^{d} and R^{e} is H or OH, and the other is H or —OH; or R^{d} and R^{e} together with the carbon to which they are attached form C═O, C═C(R)₂, or C═N—OR^{k};

One of R^{f} and R^{g} is H, and the other is H or halogen;

R^{j} is the same or different on each occurrence and is H or halogen;

R^{k} is amino(C₁-C₆)alkyl or (C₁-C₆)alkyl;

or a tautomer, stereoisomer, Z and/or E isomer, optical isomer, N-oxide, hydrate, solvate, polymorph, pharmaceutically acceptable ester, amide, salt, prodrug, and isotopic derivative thereof;

provided that

when R^{a} is methyl and R^{f} and R^{g} are both hydrogen, one of R^{b} and R^{c} must be an optionally substituted aryl or optionally substituted heteroaryl.

In one embodiment, both of A and X are H. In another embodiment, both of U and Y are H.

In one embodiment, both of R^{f} and R^{g} are H. In another embodiment, one of R^{f} and R^{g} is H, the other is halogen.

R^{a} can be, for example, methyl, —CH₂OH, or —CH₂OC(O)CH₃.

One embodiment provides that R^{d} and R^{e} are both H. Other embodiments provide that R^{d} and R^{e} together with the carbon to which they are attached form C═O, C═CH₂, or C═N—OR^{k}. R^{K} can be, for example, methyl. In certain embodiments, R^{b} and R^{c} are both H; or one of R^{b} and R^{c} is H, and the other is optionally substituted aryl.

Exemplified compounds of the invention include a compound selected from the group consisting of:
16-Fluoro-10,13-dimethyl-tetradecahydro-cyclopenta[a]phenanthrene-3,17-dione 3-(O-pyrrolidin-3-yl-oxime):

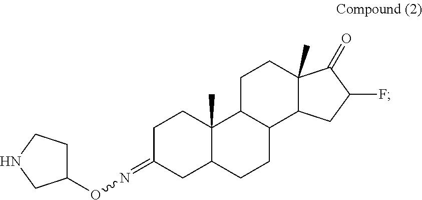 [SMILES: C[C@]12CCC(=NOC3CCNC3)CC1CCC1C2CC[C@]2(C)C(=O)C(F)CC12]

16-Fluoro-10,13-dimethyl-tetradecahydro-cyclopenta[a]phenanthrene-3,6,17-trione 3-(O-pyrrolidin-3-yl-oxime):

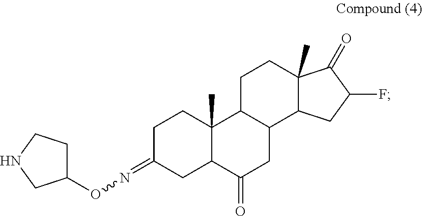 [SMILES: C[C@]12CCC(=NOC3CCNC3)CC1C(=O)CC1C2CC[C@]2(C)C(=O)C(F)CC12]

16-Fluoro-10,13-dimethyl-6-methylene-tetradecahydro-cyclopenta[a]phenanthrene-3,17-dione 3-(O-pyrrolidin-3-yl-oxime)

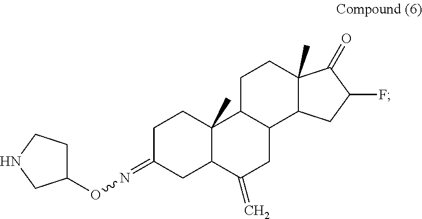 [SMILES: C=C1CC2C(CC[C@]3(C)C(=O)C(F)CC23)[C@@]2(C)CCC(=NOC3CCNC3)CC12]

16-Fluoro-10,13-dimethyl-tetradecahydro-cyclopenta[a]phenanthrene-3,6,17-trione 6-(O-methyl-oxime) 3-(O-pyrrolidin-3-yl-oxime):

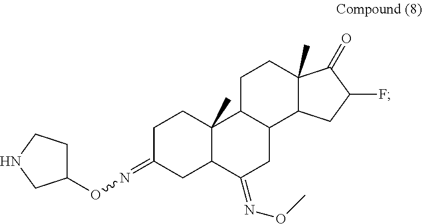 [SMILES: CO/N=C1\CC2C(CC[C@]3(C)C(=O)C(F)CC23)[C@@]2(C)CCC(=NOC3CCNC3)CC12]

10,13-Dimethyl-4-phenyl-tetradecahydro-cyclopenta[a]phenanthrene-3,17-dione 3-(O-pyrrolidin-3-yl-oxime):

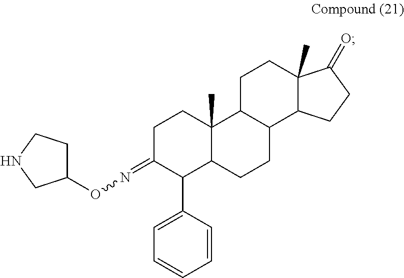 [SMILES: C[C@]12CCC(=NOC3CCNC3)C(c3ccccc3)C1CCC1C2CC[C@]2(C)C(=O)CCC12]

10-Hydroxymethyl-13-methyl-tetradecahydro-cyclopenta[a]phenanthrene-3,17-dione 3-(O-pyrrolidin-3-yl-oxime):

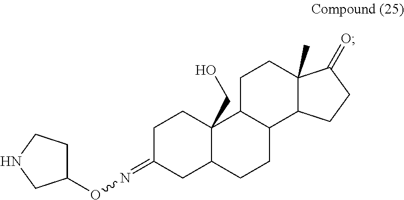 [SMILES: C[C@]12CCC3C(CCC4CC(=NOC5CCNC5)CC[C@@]43CO)C1CCC2=O]

Acetic acid 13-methyl-17-oxo-3-(pyrrolidin-3-yloxyimino)-hexadecahydro-cyclopenta[a]phenanthren-10-ylmethyl ester:

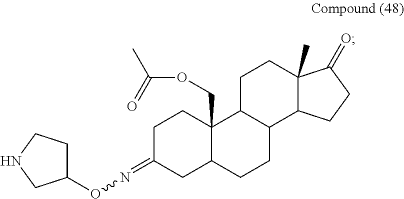 [SMILES: CC(=O)OC[C@]12CCC(=NOC3CCNC3)CC1CCC1C2CC[C@]2(C)C(=O)CCC12]

10-Hydroxymethyl-13-methyl-tetradecahydro-cyclopenta[a]phenanthrene-3,6,17-trione 3-(O-pyrrolidin-3-yl-oxime):

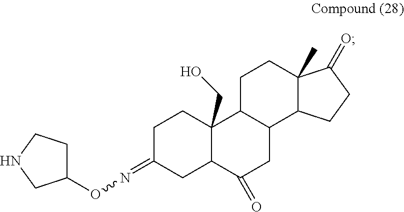 [SMILES: C[C@]12CCC3C(CC(=O)C4CC(=NOC5CCNC5)CC[C@@]43CO)C1CCC2=O]

and
10-Hydroxymethyl-13-methyl-tetradecahydro-cyclopenta[a]phenanthrene-3,6,17-trione 6-(O-methyl-oxime) 3-(O-pyrrolidin-3-yl-oxime):

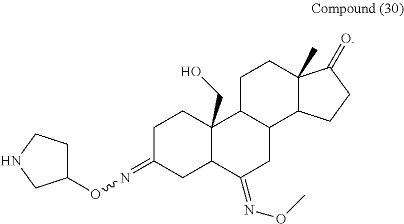 [SMILES: CO/N=C1\CC2C(CC[C@]3(C)C(=O)CCC23)[C@@]2(CO)CCC(=NOC3CCNC3)CC12]

In one embodiment, U and Y together with the carbon to which they are attached to form C═O. In one embodiment, A is halogen and X is H. Certain exemplified compounds are provided infra.

In another embodiment, both of U and Y are H. In one embodiment, A is H, and X is F. Certain exemplified compounds are provided infra.

In yet another embodiment, one of U and Y is H, and the other is halogen. In one instance, both of A and X are H (see below for the exemplified compounds).

In related embodiments, the invention also provides compounds of Formula (IIa):

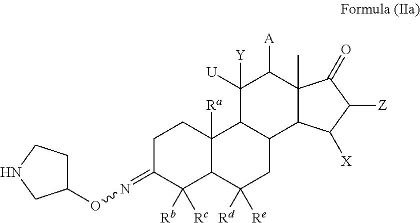

wherein:

A and X, for each occurrence, independently is H or halogen;

One of U and Y is H, and the other is H or halogen; or U and Y together with the carbon to which they are attached form C═O;

Z is halogen;

R^{a} is H, or (C₁-C₆)alkyl that is further optionally substituted by hydroxyl or —OC(O)—(C₁-C₆)alkyl;

One of R^{b} and R^{c} is H, or halogen, and the other is H, halogen, optionally substituted aryl, optionally substituted heteroaryl, alkyl, or OH;

One of R^{d} and R^{e} is H or OH, and the other is H or —OH; or R^{d} and R^{e} together with the carbon to which they are attached form C═O, C═C(R^{j})₂, or C═N—OR^{k};

R^{j} is the same or different on each occurrence and is H or halogen;

R^{k} is amino(C₁-C₆)alkyl or (C₁-C₆)alkyl; or a tautomer, stereoisomer, Z and/or E isomer, optical isomer, N-oxide, hydrate, solvate, polymorph, pharmaceutically acceptable ester, amide, salt, prodrug, and isotopic derivative thereof;

provided that

when R^{a} is methyl and R^{f} and R^{g} are both hydrogen, one of R^{b} and R^{c} must be an optionally substituted aryl or optionally substituted heteroaryl.

In certain embodiments, Z is fluorine. Examples of the such compounds of Formula (IIa) include, but are not limited to, Compound Nos. 2, 4, 6, 8, 64, 65 and 66, supra.

In another aspect, the invention provides a method for reducing the growth, proliferation or survival of a neoplastic cell. The method includes contacting the cell with an effective amount of a compound of the invention. The compound reduces the growth, proliferation or survival of a neoplastic cell.

In one embodiment, the method further comprises selecting the compound for binding to a membrane androgen receptor or for competing with a ligand for binding to said receptor.

In a separate embodiment, neoplasia is a solid tumor or hematological cancer. Yet another embodiment provides that the neoplastic cell is derived from a tissue selected from the group consisting of lung, breast, CNS, colon, prostate, ovary, pancreas, kidney and melanoma.

In one embodiment, the cell expresses MDR-1 or P-glycoprotein.

Yet another aspect of the invention provides a method of inducing cell death in a neoplastic cell. The method includes contacting the cell with a therapeutically effective amount of a compound of the invention, which thereby induces cell death. In one embodiment, the cell is in a subject. Another embodiment provides that the cell death is apoptotic cell death.

A further aspect of this invention provides a method of preventing or treating a neoplasia in a subject. This method includes administering to a subject in need thereof a therapeutically effective amount of a compound of the invention, which thereby prevents or treats neoplasia in the subject.

One embodiment provides that the subject is a mammal. Another embodiment provides that the subject is a human patient.

In certain embodiments, the methods of the invention reduce the growth or proliferation of a neoplasia in a subject. The neoplasia recited in the methods of the invention can be, but is not limited to, a lung, breast, CNS, colon, prostate, ovary, pancreas, kidney or skin cancer.

In some instances, the neoplasia is resistant to one or more therapeutic agents. In other instances, the neoplasia is multidrug resistant. Certain embodiments provide that the neoplasia has alterations in the expression or activity of an ABC transporter, tubulin, or topoisomerase polypeptide or polynucleotide. In other embodiments, the neoplasia has an increase in the expression or activity of MDR1 or P-glycoprotein.

In another aspect, the invention provides a method for the treatment of a subject having a multidrug resistant or refractory neoplasia. This method includes administering to the subject in need thereof a therapeutically effective amount of a compound of the invention, which thereby treats a subject having a multidrug resistant or refractory neoplasia. In one embodiment, the subject is a human patient. In a separate embodiment, the method reduces the growth or proliferation of the neoplasia. In another embodiment, the method induces the death of a neoplastic cell.

In one embodiment, the neoplasia is resistant to one or more therapeutic agents. In another embodiment, the neoplasia has alterations in the expression or activity of an ABC transporter, tubulin, or topoisomerase polypeptide or polynucleotide. Other embodiments provide that the neoplasia has an increase in the expression or activity of MDR1 or P-glycoprotein.

The method may further comprise administering a compound selected from the group consisting of vinca alkaloids, taxanes, epothilones, antifolates, purine analogs, pyrimidine analogs, DNA intercalators, topoisomerase inhibitors, topotecan, alkylating agents, platinum-based agents, receptor antagonists, hormone agents, anthracyclines, epipodophyllotoxins, antibiotics, antimicrotubule drugs, protein synthesis inhibitors, toxic peptides, enzyme inhibitors and anti-mitotics. The method may also treat a patient having an end-stage disease.

This invention also provides a composition for the treatment of a neoplasia. The composition includes a therapeutically effective amount of a compound of the invention and a pharmaceutically acceptable excipient or carrier. In one embodiment, the composition further includes a therapeutically effective amount of a chemotherapeutic compound. Examples of the chemotherapeutic compounds include, but are not limited to, vinca alkaloids, taxanes, epothilones, antifolates, purine analogs, pyrimidine analogs, DNA intercalators, topoisomerase inhibitors, topotecan, alkylating agents, platinum-based agents, receptor antagonists, hormone agents, anthracyclines, epipodophyllotoxins, antibiotics, antimicrotubule drugs, protein synthesis inhibitors, toxic peptides, enzyme inhibitors and anti-mitotics. In another embodiment, the composition further includes a therapeutically effective amount of a taxane.

A further aspect of this invention provides a packaged pharmaceutical for the treatment of neoplasia. The packaged pharmaceutical includes a therapeutically effective amount of a compound of the invention, and written instructions for administration of the compound. A therapeutically effective amount of one or more chemotherapeutic compounds may be also included in the packaged pharmaceutical. Examples of such chemotherapeutic compounds that may be included are listed supra. In one instance, the chemotherapeutic compound is a taxane.

In another aspect, the invention provides a method of preventing or treating a neoplasia (e.g., a membrane androgen positive solid tumor or hematological malignancy) in a subject (e.g., mouse, dog, human) by administering to the subject an effective amount of a compound of the invention. It is believed that compounds of the invention act as Na⁺K⁺ ATPase inhibitors that inhibit ligand binding to a membrane androgen receptor, thereby preventing or treating the neoplasia. In one embodiment, the Na⁺K⁺ ATPase inhibitor binds a Na⁺K⁺ ATPase and inhibits Na⁺K⁺ ATPase activity. In another embodiment, the Na⁺K⁺ ATPase inhibitor binds to a membrane androgen receptor and competitively inhibits ligand binding to the receptor. In another embodiment, the Na⁺K⁺ ATPase inhibitor induces cell death in a neoplastic cell of the neoplasia. In one embodiment, the neoplasia is a membrane androgen positive solid tumor or hematological malignancy. In still other embodiments, the neoplasia is a prostate cancer, breast cancer, or colon cancer.

In certain embodiments, compounds for use in these aspects of the invention include:
16-Fluoro-10,13-dimethyl-tetradecahydro-cyclopenta[a]phenanthrene-3,17-dione 3-(O-pyrrolidin-3-yl-oxime):

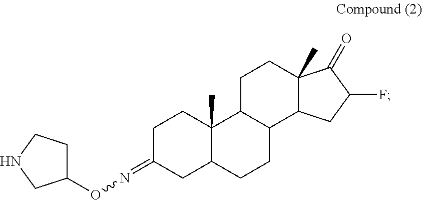 [SMILES: C[C@]12CCC(=NOC3CCNC3)CC1CCC1C2CC[C@]2(C)C(=O)C(F)CC12]

16-Fluoro-10,13-dimethyl-tetradecahydro-cyclopenta[a]phenanthrene-3,6,17-trione 3-(O-pyrrolidin-3-yl-oxime):

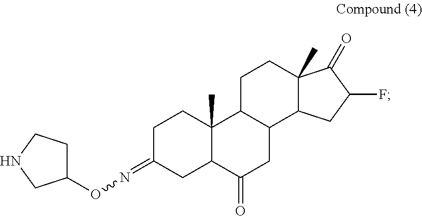 [SMILES: C[C@]12CCC(=NOC3CCNC3)CC1C(=O)CC1C2CC[C@]2(C)C(=O)C(F)CC12]

16-Fluoro-10,13-dimethyl-6-methylene-tetradecahydro-cyclopenta[a]phenanthrene-3,17-dione 3-(O-pyrrolidin-3-yl-oxime)

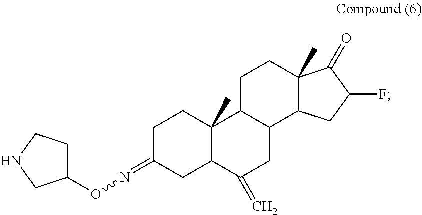 [SMILES: C=C1CC2C(CC[C@]3(C)C(=O)C(F)CC23)[C@@]2(C)CCC(=NOC3CCNC3)CC12]

16-Fluoro-10,13-dimethyl-tetradecahydro-cyclopenta[a]phenanthrene-3,6,17-trione 6-(O-methyl-oxime) 3-(O-pyrrolidin-3-yl-oxime):

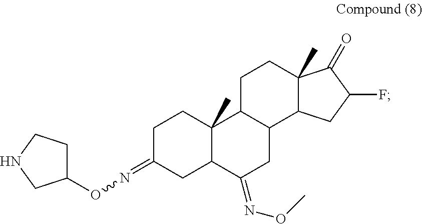 [SMILES: CO/N=C1\CC2C(CC[C@]3(C)C(=O)C(F)CC23)[C@@]2(C)CCC(=NOC3CCNC3)CC12]

10,13-Dimethyl-4-phenyl-tetradecahydro-cyclopenta[a]phenanthrene-3,17-dione 3-(O-pyrrolidin-3-yl-oxime)

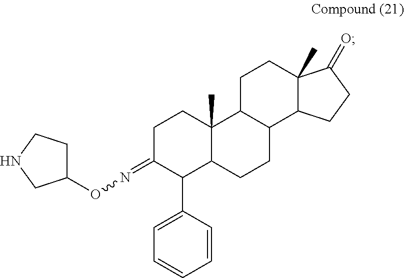 [SMILES: C[C@]12CCC(=NOC3CCNC3)C(c3ccccc3)C1CCC1C2CC[C@]2(C)C(=O)CCC12]

10-Hydroxymethyl-13-methyl-tetradecahydro-cyclopenta[a]phenanthrene-3,17-dione 3-(O-pyrrolidin-3-yl-oxime):

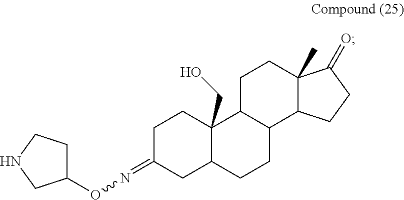 [SMILES: C[C@]12CCC3C(CCC4CC(=NOC5CCNC5)CC[C@@]43CO)C1CCC2=O]

Acetic acid 13-methyl-17-oxo-3-(pyrrolidin-3-yloxyimino)-hexadecahydro-cyclopenta[a]phenanthren-10-ylmethyl ester:

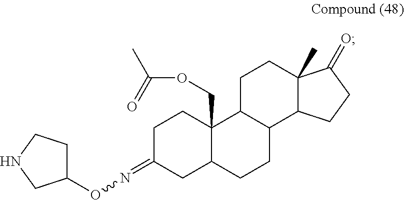 [SMILES: CC(=O)OC[C@]12CCC(=NOC3CCNC3)CC1CCC1C2CC[C@]2(C)C(=O)CCC12]

10-Hydroxymethyl-13-methyl-tetradecahydro-cyclopenta[a]phenanthrene-3,6,17-trione 3-(O-pyrrolidin-3-yl-oxime):

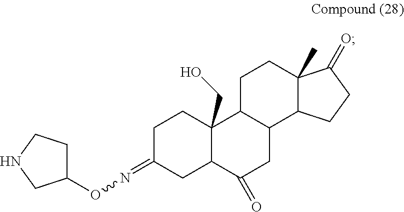 [SMILES: C[C@]12CCC3C(CC(=O)C4CC(=NOC5CCNC5)CC[C@@]43CO)C1CCC2=O]

10-Hydroxymethyl-13-methyl-tetradecahydro-cyclopenta[a]phenanthrene-3,6,17-trione 6-(O-methyl-oxime) 3-(O-pyrrolidin-3-yl-oxime):

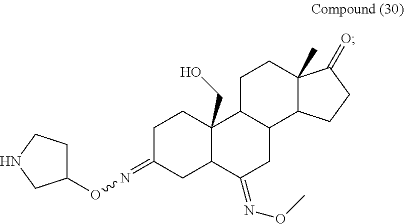 [SMILES: CO/N=C1\CC2C(CC[C@]3(C)C(=O)CCC23)[C@@]2(CO)CCC(=NOC3CCNC3)CC12]

and
16-Fluoro-6,10,13-trimethyl-1,6,7,8,9,10,11,12,13,14,15,16-dodecahydro-2H-cyclopenta[a]phenanthrene-3,17-dione 3-(O-pyrrolidin-3-yl-oxime):

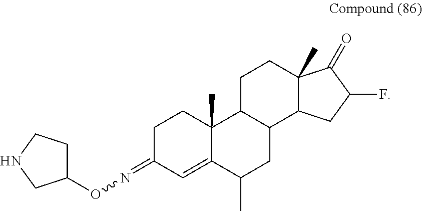 [SMILES: CC1CC2C(CC[C@]3(C)C(=O)C(F)CC23)[C@@]2(C)CCC(=NOC3CCNC3)C=C12]

In yet another aspect, the invention provides a method for treating or preventing prostate cancer in a subject. The method involves administering to the subject an effective amount of a compound of the invention that is capable of binding and inhibiting a Na⁺K⁺ ATPase. In certain embodiment, the compound is further capable of competitively inhibiting ligand binding to the membrane androgen receptor on a prostate cancer cell. In one embodiment, the method induces cell death (e.g., apoptosis) in a cell of the prostate cancer. In another embodiment, the compound binds the membrane androgen receptor. In certain embodiments, compounds for use in this aspect of the invention include compound Nos. 2, 4, 6, 8, 21, 25, 48, 28, 30, and 86 shown above.

In still another aspect, the invention provides methods for treating neoplasia through

administering to the subject a therapeutically effective amount of a compound of the invention. It is believed that the compound inhibits a Na⁺K⁺ ATPase and binds to a membrane androgen receptor, thereby preventing or treating the neoplasia (such as, a prostate cancer). The compound may further induce cell death in a neoplastic cell of said neoplasia.

The invention further provides compositions for the treatment or prevention of a neoplasia. The compositions include a therapeutically effective amount of a compound of the invention, and a pharmaceutically acceptable excipient. The compound may further competitively inhibit ligand binding to the membrane androgen receptor on a neoplastic cell. In certain embodiments, compounds for use in this aspect include compound Nos. 2, 4, 6, 8, 21, 25, 48, 28, 30, and 86 as shown above.

The invention further provides packaged pharmaceuticals for the treatment of neoplasia. Compositions and articles defined by the invention were isolated or otherwise manufactured in connection with the examples provided below. Other features and advantages of the invention will be apparent from the detailed description, and from the claims.

### BRIEF DESCRIPTION OF THE DRAWINGS

FIGS. 1A-1F are micrographs showing 4′,6-diamidino-2-phenylindole (DAPI) and membrane androgen receptor immunofluorescent staining in DU145 cells obtained with testosterone-human serum albumin-Fluorescein isothiocyanate (TAC-FITC) conjugates with or without treatment with compound 2. Membrane fluorescence was largely reduced in compound 2 pre-treated cells, which indicates that compound 2 precludes binding of TAC-FITC to the membrane androgen receptor.

### DETAILED DESCRIPTION OF THE INVENTION

The invention features compounds delineated herein and methods of using such compounds for the treatment or prevention of neoplasia. In particular embodiments, the compounds of the invention are useful for the treatment of multidrug resistant neoplasia.

The invention is based, at least in part, on the discovery that a compound of the invention has potent anti-neoplastic activity in vitro. In certain instances, a compound of the invention is a Na+/K+ ATPase inhibitor that also has potent anti-neoplastic activity. As shown herein below, Compound Nos. 2, 4, 6, 8, 21, 25, 28, 30, 48 and 86 reduced the viability and/or cell proliferation of cell lines representative of lung, breast, CNS, prostate, ovarian, colon and renal neoplasias.

The invention also provides a number of targets that are useful for the development of highly specific drugs to treat or prevent a disorder or disease characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

### DEFINITIONS

Before a further description of the present invention, and in order that the invention may be more readily understood, certain terms are first defined and collected here for convenience.

The term “administration” or “administering” includes routes of introducing a compound(s) to a subject to perform their intended function. Examples of routes of administration that can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, topical by lotion or ointment; and rectal by suppositories. Oral administration is preferred. The injection can be bolus or can be continuous infusion. Depending on the route of administration, the compound can be coated with or disposed in a selected material to protect it from natural conditions which may detrimentally effect its ability to perform its intended function.

The compound can be administered alone, or in conjunction with either another agent as described above (e.g. another chemotherapeutic agent) or with a pharmaceutically-acceptable carrier, or both. The compound can be administered prior to the administration of the other agent, simultaneously with the agent, or after the administration of the agent. Furthermore, the compound can also be administered in a proform which is converted into its active metabolite, or more active metabolite in vivo.

By “ABC transporter activity” is meant the ATP-dependent directed translocation of substrates across membranes. Methods for assaying ABC transporter activity are known in the art. See, for example, Glavinas et al., Current Drug Delivery, 2004, Vol. 1, No. I 33, which is incorporated by reference.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The term “alkoxy,” as used herein, refers to an alkyl or a cycloalkyl group which is linked to another moiety though an oxygen atom. Alkoxy groups can be optionally substituted with one or more substituents.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “ameliorate” means to decrease, suppress, attenuate, diminish, arrest, or stabilize the development or progression of a disease.

The term “alteration” refers to a change (increase or decrease) in a parameter as detected by standard art known methods, such as those described herein.

The term “aryl” refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like.

Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “cancer” refers to a malignant tumor of potentially unlimited growth that expands locally by invasion and systemically by metastasis.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “chiral” refers to molecules which have the property of non-superimposability of the mirror image partner, while the term “achiral” refers to molecules which are superimposable on their mirror image partner.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. “Detect” refers to identifying the presence, absence or amount of the object to be detected.

By “disease” is meant any condition or disorder that damages or interferes with the normal function of a cell, tissue, or organ.

The term “diastereomers” refers to stereoisomers with two or more centers of dissymmetry and whose molecules are not mirror images of one another.

The term “effective amount” refers to the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

A therapeutically effective amount of a compound delineated herein (i.e., an effective dosage) may range from about 0.1 g to 20 milligram per kilogram of body weight per day (mg/kg/day) (e.g., 0.1 μg/kg to 2 mg/kg, 0.3-3 μg/kg, 0.18-0.54 mg/kg). In other embodiments, the amount varies from about 0.1 mg/kg/day to about 100 mg/kg/day. In still other embodiments, the amount varies from about 0.001 g to about 100 μg/kg (e.g., of body weight). One of skill in the art can readily extrapolate from dosages shown to be effective in in vivo testing to dosages that are likely to be effective in humans. In one embodiment, about 0.1-200 mg/kg/day a compound of the invention (e.g., any of compound Nos. 2, 4, 6, 8, 21, 25, 28, 48 and 30) is administered to a mouse, preferably 1-100 mg/kg, more preferably 5-50 mg/kg. In another embodiment, a dog receives 1-20 mg/kg of such compounds. In another embodiment, a human subject receives 0.1 μg/kg to 2 mg/kg of a compound of the invention (e.g., any of compounds 2, 4, 6, 8, 21, 25, 28, 48 and 30) per day. In yet another embodiment, 0.3-3 μg/kg of such compounds is administered to a human subject. In still another embodiment, 0.18-0.54 mg/kg total per day is administered to a human subject. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound delineated herein can include a single treatment or, preferably, can include a series of treatments. In one example, a subject is treated with a compound delineated herein in the range of between about 0.1 g to 20 milligram per kilogram of body weight per day (mg/kg/day) (e.g., 0.1 μg/kg to 2 mg/kg, 0.3-3 μg/kg, 0.18-0.54 mg/kg). In other embodiments, the amount varies from about 0.1 mg/kg/day to about 100 mg/kg/day. In still other embodiments, the amount varies from about 0.001 g to about 100 μg/kg (e.g., of body weight). If desired, the dosage is administered one time per day, two times per day, or one time per week. Treatment is carried out for between about 1 to 10 weeks, preferably between 2 to 8 weeks, more preferably between about 3 to 7 weeks, and even more preferably for about 4, 5, or 6 weeks. It will also be appreciated that the effective dosage of a compound delineated herein used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, with said heteroatoms selected from O, N, and S, and the remainder ring atoms being carbon. Heteroaryl groups may be optionally substituted with one or more substituents. Examples of heteroaryl groups include, but are not limited to, pyridyl, furanyl, benzodioxolyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, and indolyl. In one embodiment of the invention, heteroaryl refers to thienyl, furyl, pyridyl, or indolyl.

The term “heterocyclic” as used herein, refers to organic compounds that contain at least at least one atom other than carbon (e.g., S, O, N) within a ring structure. The ring structure in these organic compounds can be either aromatic or non-aromatic. Some examples of heterocyclic moieties include, are not limited to, pyridine, pyrimidine, pyrrolidine, furan, tetrahydrofuran, tetrahydrothiophene, and dioxane.

By “inhibit Na⁺K⁺ ATPase” is meant detectably reduce the enzymatic activity of a Na⁺K⁺ ATPase. Methods for assaying Na⁺K⁺ ATPase activity are known in the art and described herein at Example 10.

The term “isomers” or “stereoisomers” refers to compounds which have identical chemical constitution, but differ with regard to the arrangement of the atoms or groups in space.

The term “isotopic derivatives” includes derivatives of compounds in which one or more atoms in the compounds are replaced with corresponding isotopes of the atoms.

For example, an isotopic derivative of a compound containing a carbon atom (C¹²) would be one in which the carbon atom of the compound is replaced with the C¹³ isotope.

The term “multidrug resistant” refers to a neoplasia that has increased MDR1 expression or activity relative to a reference neoplasia, or that fails to respond to a chemotherapeutic agent or has reduced susceptibility to one or more chemotherapeutic agents relative to a reference neoplasia. In one embodiment, the response to the chemotherapeutic agent is reduced by at least about 10%, 25%, 50%, 75% 95% or more.

The term “P-glycoprotein polypeptide” refers to a protein having at least about 85% or more amino acid identity to NCBI Accession No. CAA41558 or a fragment thereof that has ABC transporter activity.

The term “MDR1 polynucleotide” refers to a nucleic acid sequence encoding a P-glycoprotein polypeptide.

The term “neoplastic” refers to those cells having the capacity for autonomous growth, i.e., an abnormal state or condition characterized by rapidly proliferating cell growth. A neoplastic disease state may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The language “inhibiting the growth” of the neoplasm includes the slowing, interrupting, arresting or stopping its growth and metastases and does not necessarily indicate a total elimination of the neoplastic growth.

The term “modulate” refers to increases or decreases in a parameter in response to exposure to a compound of the invention.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the term neoplasia generally refers to cells experiencing abnormal cell growth rates. Neoplasias include “tumors,” which may be either benign, premalignant or malignant.

The term “obtaining” as in “obtaining compound” is intended to include purchasing, synthesizing or otherwise acquiring the compound.

The term “optical isomers” as used herein includes molecules, also known as chiral molecules, are exact non-superimposable mirror images of one another.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “polymorph” as used herein, refers to solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Certain prodrug moieties are, for example, propionoic acid esters and acyl esters. Prodrugs which are converted to active forms through other mechanisms in vivo are also included.

The language “a prophylactically effective anti-neoplastic amount” of a compound refers to an amount of a compound delineated herein or otherwise described herein which is effective, upon single or multiple dose administration to the patient, in preventing or delaying the occurrence of the onset of a neoplastic disease state.

Furthermore, the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Both configurations, cis/trans and/or Z/E are encompassed by the compounds of the invention.

With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

By “reference” is meant a standard or control condition.

By “refractory neoplasia” is meant a neoplasia resistant to treatment with standard chemotherapeutic agents.

The term “subject” includes organisms which are capable of suffering from a neoplasia or who could otherwise benefit from the administration of a compound of the invention, such as human and non-human animals. Preferred human animals include human patients suffering from or prone to suffering from a neoplasia, as described herein. The term “non-human animals” of the invention includes all vertebrates, e.g., mammals, e.g., rodents, e.g., mice, and non-mammals, such as, non-human primates, also sheep, dog, cow, chickens, amphibians, and reptiles.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

As used herein, the term “tautomers” refers to isomers of organic molecules that readily interconvert by tautomerization, in which a hydrogen atom or proton migrates in the reaction, accompanied in some occasions by a switch of a single bond and an adjacent double bond.

The structures of the compounds of the invention may include asymmetric carbon atoms. Accordingly, the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases, such as brucine, quinine, ephedrine, strychnine, and the like. Alternatively, diastereomeric esters can be formed with enantiomerically pure chiral alcohols, such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### Na⁺/K⁺-ATPase, or Na⁺/K⁺ Pump

The Na⁺/K⁺-ATPase, or Na⁺/K⁺ pump is a complex of integral membrane proteins that actively transports sodium and potassium ions across the cell plasma membrane. In addition to pumping ions across the membrane, the enzyme functions as a receptor for cardiac glycosides, such as ouabain, digoxin, marinobufagenin and others (reviewed in Mijatovic T et al., Biochim Biophys Acta. 2007; 1776:32-57). Surprisingly, compounds of the invention were discovered to have Na⁺/K⁺-ATPase inhibitory activity.

### Compounds of the Invention

Novel compounds of the invention specifically exclude compounds of the prior art, including those disclosed and/or claimed in WO 2009/047101, PCT/EP2010/058842, and PCT/EP2010/058843. Accordingly, the invention contemplates one or more subgenuses of compounds of Formula I and/or Formula Ia described herein resulting from the exclusion of one of one or more compounds of the prior art, including those disclosed and/or claimed in WO 2009/047101, PCT/EP2010/058842, and PCT/EP2010/058843.

In one aspect, the invention provides a compound of Formula (I):

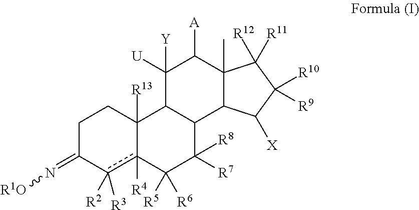

wherein:

[symbol] represents a single bond or double bond in the ring structure of Formula (I);

A and X, for each occurrence, independently is H or halogen;

One of U and Y is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸;

or U and Y together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;

R¹ is aminoalkyl, alkyl, or a 4- to 6-membered heterocyclic ring, wherein said aminoalkyl is optionally substituted by one or more alkyl or acetyl; said alkyl, for each occurrence, independently is optionally substituted by guanidinyl, heteroaryl, or a 4- to 6-membered heterocyclic ring; and each 4- to 6-membered heterocyclic ring independently is optionally substituted by one or more alkyl;

One of R² and R³ is H, halogen, optionally substituted (C₁-C₆)alkyl, OH, or absent, and the other is H, halogen, optionally substituted aryl, optionally substituted heteroaryl, (C₁-C₆)alkyl, OH, or absent;

or R² and R³ together with the carbon to which they are attached form cycloalkyl;

R⁴ is H, —OH, or absent;

One of R⁵ and R⁶ is H, OH, or halogen, and the other is H; —OR¹⁴; alkyl optionally substituted with hydroxyl or alkoxy; —C(O)—NH₂; —C(O)—O-alkyl; —NHR¹⁵; alkynyl; or R⁵ and R⁶ together with the carbon to which they are attached form C═O, C═C(R¹⁶)₂, or C═N—OR²⁰;

One of R⁷ and R⁸ is H, or halogen, and the other is H; —OR¹⁴; alkyl optionally substituted with hydroxyl or alkoxy; —C(O)—NH₂; —C(O)—O-alkyl; —NHR¹⁵; alkynyl; or R⁷ and R⁸ together with the carbon to which they are attached form C═O, C═C(R¹⁶)₂, C═N—OR²⁰, or a cyclopropyl ring;

One of R⁹ and R¹⁰ is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸; or R⁹ and R¹⁰ together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;

One of R¹¹ and R¹² is H, and the other is —OH; alkoxy optionally substituted with a hydroxyl or an amino group; optionally substituted heterocyclyl; optionally substituted heteroaryl; or —OC(O)R¹⁹; or R¹¹ and R¹² together with the carbon atom to which they are attached form C═O;

R¹³ is H, —CH₂OR¹⁷, —CHO, —COOR¹⁷, —COR¹⁷, —C≡C—R¹⁷, —C(O)NR¹⁷R¹⁸, —(C₁-C₆)alkyl, or a heteroaryl, wherein said (C₁-C₆)alkyl is further optionally substituted by one or more substituents selected from the group of (C₁-C₄)alkyl, hydroxyl, halogen, alkoxy, aryl, —C(O)—NR¹⁷R¹⁸, —OC(O)-alkyl, —C(O)—O-alkyl, cycloalkyl, heteroaryl, and —NR¹⁷R¹⁸; and said heteroaryl is further optionally substituted by (C₁-C₄)alkyl, halogen, hydroxyl, alkoxy, arylalkyl, or cycloalkyl(C₁-C₄)alkyl;

R¹⁴ is H, alkyl, or —NO₂;

R¹⁵ is H or formyl;

R¹⁶, for each occurrence, is the same or different and is H or halogen;

R¹⁷ and R¹⁸, for each occurrence, independently is H, alkyl, alkenyl, alkynyl, cycloalkyl, aryl, aminocarbonyl, alkoxycarbonyl, amino, or halogen; wherein R¹⁷ and R¹⁸ independently are further optionally substituted with one or more moieties selected from the group of alkyl, alkenyl, alkynyl, amino, alkoxy, and cycloalkyl,

R¹⁹ is optionally substituted alkyl, or optionally substituted aryl;

R²⁰ is amino(C₁-C₆)alkyl, (C₁-C₆)alkyl, or a 4- to 6-membered heterocyclic ring, wherein said amino(C₁-C₆)alkyl is optionally substituted by one or more (C₁-C₆)alkyl or acetyl; each heterocyclic ring independently is optionally substituted by one or more (C₁-C₆)alkyl; and each (C₁-C₆)alkyl, independently, is optionally substituted by guanidinyl, heteroaryl, or a 4- to 6-membered heterocyclic ring;

or a tautomer, stereoisomer, Z and/or E isomer, optical isomer, N-oxide, hydrate, solvate, polymorph, pharmaceutically acceptable ester, amide, salt, prodrug, and isotopic derivative thereof;

provided that

when [symbol] represents a single bond, R¹³ is methyl, and R⁹ and R¹⁰ are both hydrogen, one of R² and R³ must be an optionally substituted aryl or optionally substituted heteroaryl; and

when [symbol] represents a double bond and R¹³ is methyl, R⁹ and R¹⁰ cannot be both hydrogen at the same time.

In one embodiment, A and X are both H. In another embodiment, both of U and Y are H.

One embodiment provides that R¹¹ and R¹² together with the carbon atom to which they are attached form C═O.

A separate embodiment provides that R¹ is amino(C₁-C₆)alkyl a 4- to 6-membered heterocyclic ring, or (C₁-C₆)alkyl substituted by a 4- to 6-membered heterocyclic ring. Examples of R¹ include, but are not limited to, aminoethyl, pyrrolidinyl, or piperidinylethyl.

One embodiment provides that the “ [symbol] ” in Formula (I) represents a single bond in the ring structure. A separate embodiment provides that the “ [symbol] ” in Formula (I) represents a double bond.

In a particular embodiment, R¹³ is unsubstituted (C₁-C₆)alkyl (e.g., methyl). In another embodiment, R¹³ is (C₁-C₆)alkyl that is substituted by hydroxyl. R¹³ may be, for example, hydroxylmethyl (i.e., —CH₂OH). In still another embodiment, R¹³ is (C₁-C₆)alkyl that is substituted by —OC(O)—(C₁-C₆)alkyl. Examples of R¹³ include, but are not limited to, —CH₂OC(O)CH₃.

In one embodiment, R⁴ is H or —OH, when [symbol] represents a single bond. In a separate embodiment, R⁴ is absent, when [symbol] represents a double bond.

One embodiment provides that one of R⁹ and R¹⁰ is H, and the other is halogen (e.g., F). In another embodiment, both of R⁹ and R¹⁰ are H.

In one embodiment, the compounds have both of R⁷ and R⁸ being H. In another embodiment, one of R⁷ and R⁸ is H, and the other is —OH. Still in another embodiment, R⁷ and R⁸ together with the carbon to which they are attached form C═O. In yet another embodiment, R⁷ and R⁸ together with the carbon to which they are attached form C═C(R¹⁶)₂. R¹⁶ may be same or different at each occurrence. Certain instances provide that R¹⁶ is the same at each occurrence and can be H or F. In a further embodiment, R⁷ and R⁸ together with the carbon to which they are attached form C═N—OR²⁰.

One embodiment of the invention provides the compounds with both of R² and R³ being H. Another embodiment provides that one of R² and R³ is H, and the other is (C₁-C₆)alkyl that is substituted or unsubstituted. For instance, one of R² and R³ is H, and the other is ethyl. Still another embodiment provides that one of R² and R³ is H, and the other is aryl that is substituted or unsubstituted. For example, one of R² and R³ is H, and the other is phenyl.

In one embodiment, the compounds of Formula (I) have both of R⁵ and R⁶ being H. In another embodiment, one of R⁵ and R⁶ is H, and the other is (C₁-C₆)alkyl, which is optionally substituted by hydroxyl or (C₁-C₄)alkoxy. For instance, one of R⁵ and R⁶ is H, and the other is methyl or hydroxymethyl (i.e., —CH₂OH). In a separate embodiment, R⁵ and R⁶ together with the carbon to which they are attached form C═O. In another embodiment, R⁵ and R⁶ together with the carbon to which they are attached form C═C(R¹⁶)₂. R¹⁶ may be the same or different for each occurrence, and can be, for example, H or F. In still another embodiment, R⁵ and R⁶ together with the carbon to which they are attached form C═N—OR²⁰. And R²⁰ is, for example, (C₁-C₆)alkyl.

Compounds of Formula (I), in accordance with the invention, include those described in Table 1 as follows:

TABLE 1

| | |
|---|---|
| 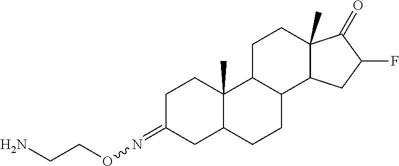 [SMILES: C[C@]12CCC(=NOCCN)CC1CCC1C2CC[C@]2(C)C(=O)C(F)CC12] | 1 |
| 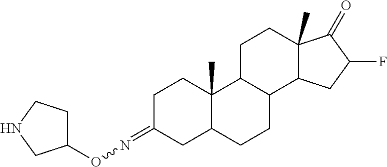 [SMILES: C[C@]12CCC(=NOC3CCNC3)CC1CCC1C2CC[C@]2(C)C(=O)C(F)CC12] | 2 |
| 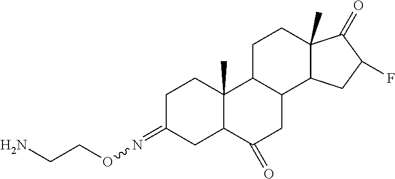 [SMILES: C[C@]12CCC(=NOCCN)CC1C(=O)CC1C2CC[C@]2(C)C(=O)C(F)CC12] | 3 |
| 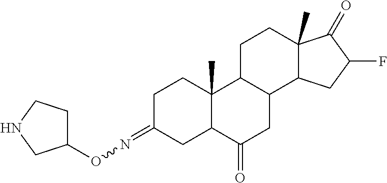 [SMILES: C[C@]12CCC(=NOC3CCNC3)CC1C(=O)CC1C2CC[C@]2(C)C(=O)C(F)CC12] | 4 |
| 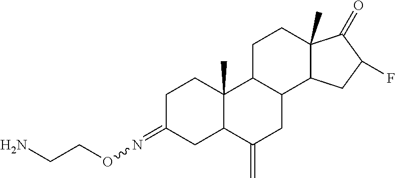 [SMILES: C=C1CC2C(CC[C@]3(C)C(=O)C(F)CC23)[C@@]2(C)CCC(=NOCCN)CC12] | 5 |
| 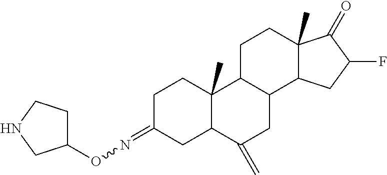 [SMILES: C=C1CC2C(CC[C@]3(C)C(=O)C(F)CC23)[C@@]2(C)CCC(=NOC3CCNC3)CC12] | 6 |
| 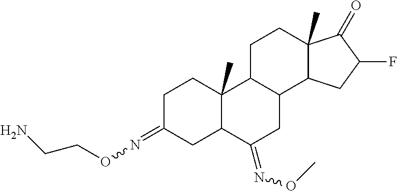 [SMILES: CON=C1CC2C(CC[C@]3(C)C(=O)C(F)CC23)[C@@]2(C)CCC(=NOCCN)CC12] | 7 |
| 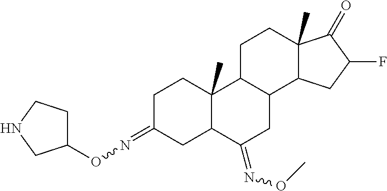 [SMILES: CON=C1CC2C(CC[C@]3(C)C(=O)C(F)CC23)[C@@]2(C)CCC(=NOC3CCNC3)CC12] | 8 |
| 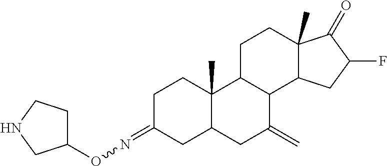 [SMILES: C=C1CC2CC(=NOC3CCNC3)CC[C@]2(C)C2CC[C@]3(C)C(=O)C(F)CC3C12] | 9 |
| 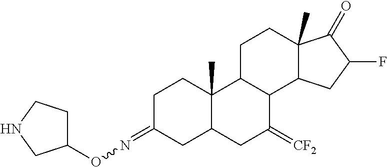 | 10 |
| 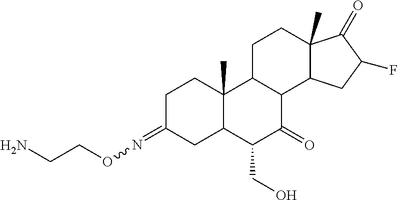 [SMILES: C[C@]12CCC(=NOCCN)CC1[C@@H](CO)C(=O)C1C2CC[C@]2(C)C(=O)C(F)CC12] | 11 |
| 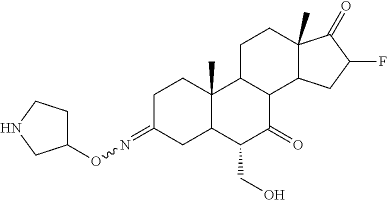 [SMILES: C[C@]12CCC(=NOC3CCNC3)CC1[C@@H](CO)C(=O)C1C2CC[C@]2(C)C(=O)C(F)CC12] | 12 |
| 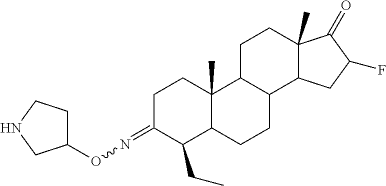 [SMILES: CC[C@H]1C(=NOC2CCNC2)CC[C@]2(C)C3CC[C@]4(C)C(=O)C(F)CC4C3CCC12] | 13 |
| 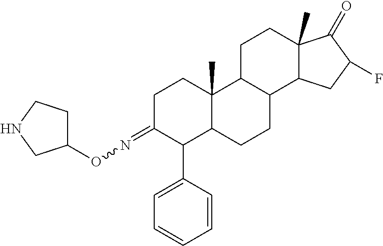 [SMILES: C[C@]12CCC(=NOC3CCNC3)C(c3ccccc3)C1CCC1C2CC[C@]2(C)C(=O)C(F)CC12] | 14 |
| 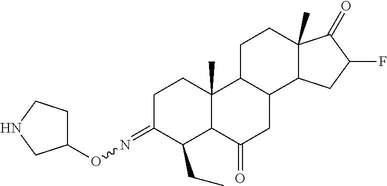 [SMILES: CC[C@H]1C(=NOC2CCNC2)CC[C@]2(C)C3CC[C@]4(C)C(=O)C(F)CC4C3CC(=O)C12] | 15 |
| 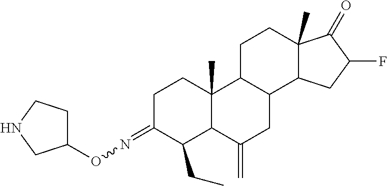 [SMILES: C=C1CC2C(CC[C@]3(C)C(=O)C(F)CC23)[C@@]2(C)CCC(=NOC3CCNC3)[C@H](CC)C12] | 16 |
| 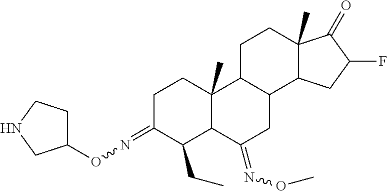 [SMILES: CC[C@H]1C(=NOC2CCNC2)CC[C@]2(C)C3CC[C@]4(C)C(=O)C(F)CC4C3CC(=NOC)C12] | 17 |
| 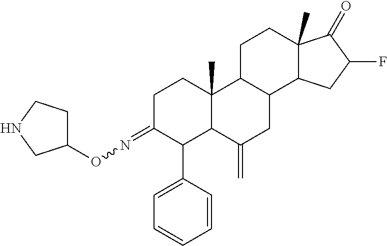 [SMILES: C=C1CC2C(CC[C@]3(C)C(=O)C(F)CC23)[C@@]2(C)CCC(=NOC3CCNC3)C(c3ccccc3)C12] | 18 |
| 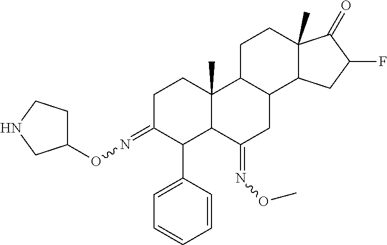 [SMILES: CON=C1CC2C(CC[C@]3(C)C(=O)C(F)CC23)[C@@]2(C)CCC(=NOC3CCNC3)C(c3ccccc3)C12] | 19 |
| 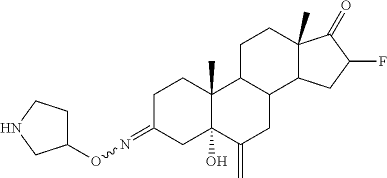 [SMILES: C=C1CC2C3CC(F)C(=O)[C@@]3(C)CCC2[C@@]2(C)CCC(=NOC3CCNC3)C[C@]12O] | 20 |
| 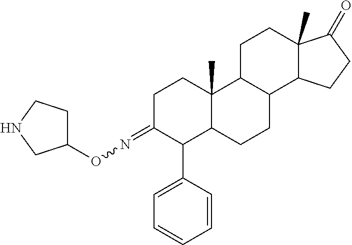 [SMILES: C[C@]12CCC(=NOC3CCNC3)C(c3ccccc3)C1CCC1C2CC[C@]2(C)C(=O)CCC12] | 21 |
| 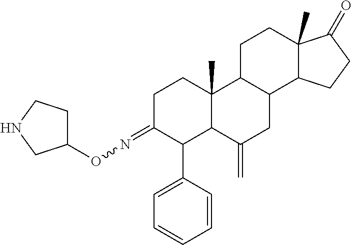 [SMILES: C=C1CC2C(CC[C@]3(C)C(=O)CCC23)[C@@]2(C)CCC(=NOC3CCNC3)C(c3ccccc3)C12] | 22 |
| 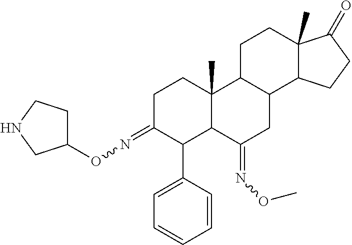 [SMILES: CON=C1CC2C(CC[C@]3(C)C(=O)CCC23)[C@@]2(C)CCC(=NOC3CCNC3)C(c3ccccc3)C12] | 23 |
| 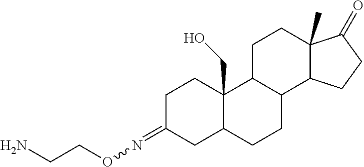 [SMILES: C[C@]12CCC3C(CCC4CC(=NOCCN)CC[C@@]43CO)C1CCC2=O] | 24 |
| 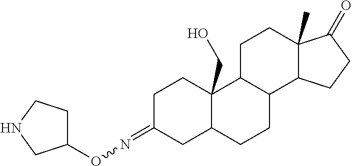 [SMILES: C[C@]12CCC3C(CCC4CC(=NOC5CCNC5)CC[C@@]43CO)C1CCC2=O] | 25 |
| 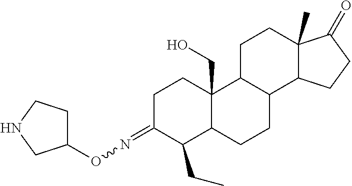 [SMILES: CC[C@H]1C(=NOC2CCNC2)CC[C@]2(CO)C3CC[C@]4(C)C(=O)CCC4C3CCC12] | 26 |
| 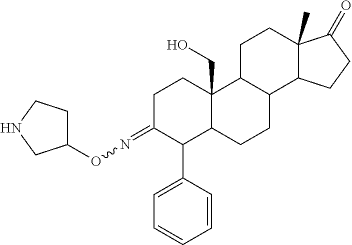 [SMILES: C[C@]12CCC3C(CCC4C(c5ccccc5)C(=NOC5CCNC5)CC[C@@]43CO)C1CCC2=O] | 27 |
| 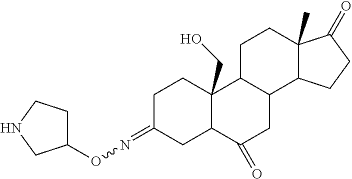 [SMILES: C[C@]12CCC3C(CC(=O)C4CC(=NOC5CCNC5)CC[C@@]43CO)C1CCC2=O] | 28 |
| 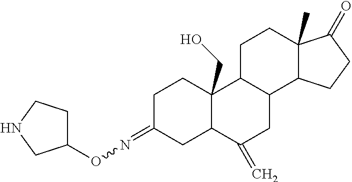 [SMILES: C=C1CC2C(CC[C@]3(C)C(=O)CCC23)[C@@]2(CO)CCC(=NOC3CCNC3)CC12] | 29 |
| 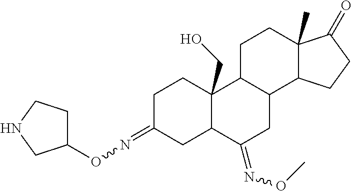 [SMILES: CON=C1CC2C(CC[C@]3(C)C(=O)CCC23)[C@@]2(CO)CCC(=NOC3CCNC3)CC12] | 30 |
| 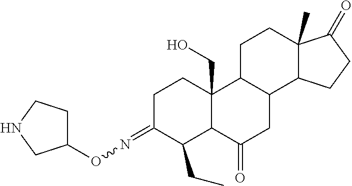 [SMILES: CC[C@H]1C(=NOC2CCNC2)CC[C@]2(CO)C3CC[C@]4(C)C(=O)CCC4C3CC(=O)C12] | 31 |
| 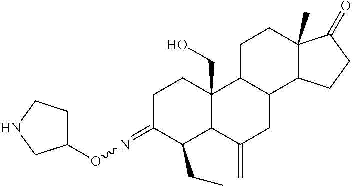 [SMILES: C=C1CC2C(CC[C@]3(C)C(=O)CCC23)[C@@]2(CO)CCC(=NOC3CCNC3)[C@H](CC)C12] | 32 |
| 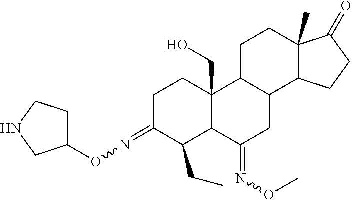 [SMILES: CC[C@H]1C(=NOC2CCNC2)CC[C@]2(CO)C3CC[C@]4(C)C(=O)CCC4C3CC(=NOC)C12] | 33 |
| 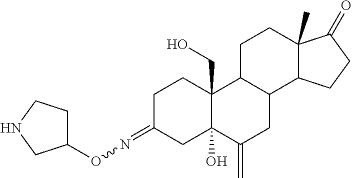 [SMILES: C=C1CC2C(CC[C@]3(C)C(=O)CCC23)[C@@]2(CO)CCC(=NOC3CCNC3)C[C@]12O] | 34 |
| 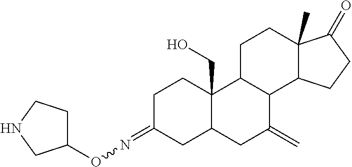 [SMILES: C=C1CC2CC(=NOC3CCNC3)CC[C@]2(CO)C2CC[C@]3(C)C(=O)CCC3C12] | 35 |
| 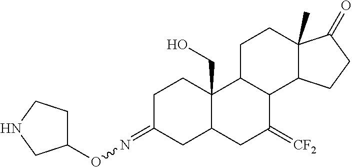 | 36 |
| 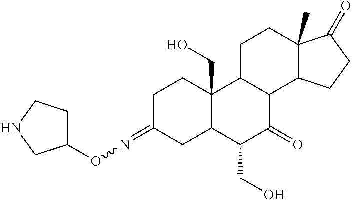 [SMILES: C[C@]12CCC3C(C(=O)[C@H](CO)C4CC(=NOC5CCNC5)CC[C@]34CO)C1CCC2=O] | 37 |
| 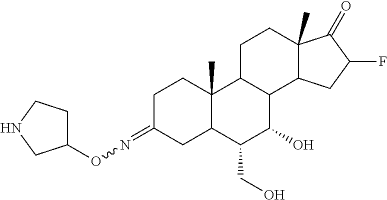 [SMILES: C[C@]12CCC(=NOC3CCNC3)CC1[C@@H](CO)[C@@H](O)C1C2CC[C@]2(C)C(=O)C(F)CC12] | 38 |
| 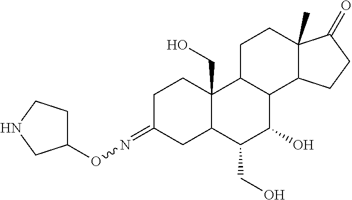 [SMILES: C[C@]12CCC3C(C1CCC2=O)[C@H](O)[C@H](CO)C1CC(=NOC2CCNC2)CC[C@]31CO] | 39 |
| 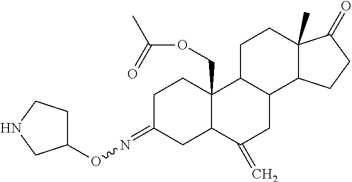 [SMILES: C=C1CC2C(CC[C@]3(C)C(=O)CCC23)[C@@]2(COC(C)=O)CCC(=NOC3CCNC3)CC12] | 40 |
| 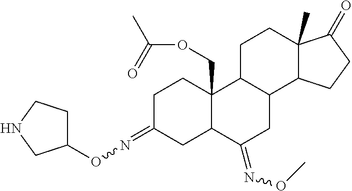 [SMILES: CON=C1CC2C(CC[C@]3(C)C(=O)CCC23)[C@@]2(COC(C)=O)CCC(=NOC3CCNC3)CC12] | 41 |
| 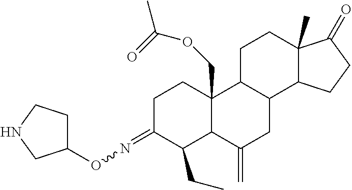 [SMILES: C=C1CC2C(CC[C@]3(C)C(=O)CCC23)[C@@]2(COC(C)=O)CCC(=NOC3CCNC3)[C@H](CC)C12] | 42 |
| 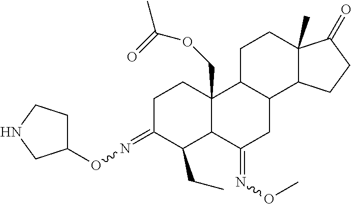 [SMILES: CC[C@H]1C(=NOC2CCNC2)CC[C@]2(COC(C)=O)C3CC[C@]4(C)C(=O)CCC4C3CC(=NOC)C12] | 43 |
| 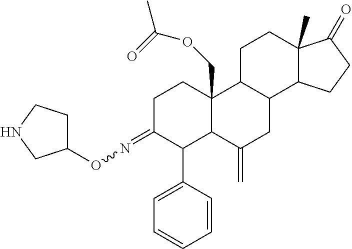 [SMILES: C=C1CC2C(CC[C@]3(C)C(=O)CCC23)[C@@]2(COC(C)=O)CCC(=NOC3CCNC3)C(c3ccccc3)C12] | 44 |
| 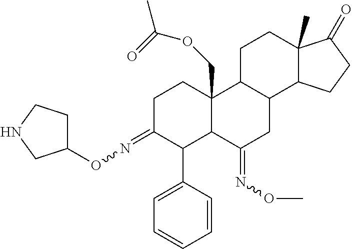 [SMILES: CON=C1CC2C(CC[C@]3(C)C(=O)CCC23)[C@@]2(COC(C)=O)CCC(=NOC3CCNC3)C(c3ccccc3)C12] | 45 |
| 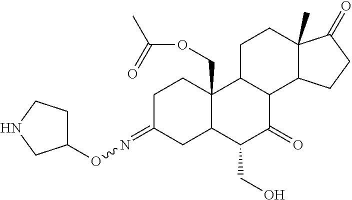 [SMILES: CC(=O)OC[C@]12CCC(=NOC3CCNC3)CC1[C@@H](CO)C(=O)C1C2CC[C@]2(C)C(=O)CCC12] | 46 |
| 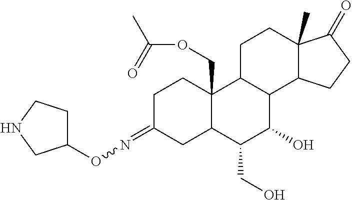 [SMILES: CC(=O)OC[C@]12CCC(=NOC3CCNC3)CC1[C@@H](CO)[C@@H](O)C1C2CC[C@]2(C)C(=O)CCC12] | 47 |
| 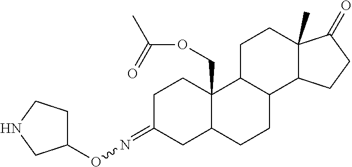 [SMILES: CC(=O)OC[C@]12CCC(=NOC3CCNC3)CC1CCC1C2CC[C@]2(C)C(=O)CCC12] | 48 |
| 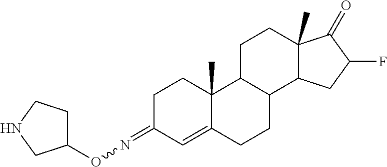 [SMILES: C[C@]12CCC(=NOC3CCNC3)C=C1CCC1C2CC[C@]2(C)C(=O)C(F)CC12] | 49 |
| 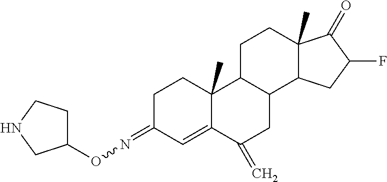 [SMILES: C=C1CC2C(CC[C@]3(C)C(=O)C(F)CC23)[C@@]2(C)CCC(=NOC3CCNC3)C=C12] | 50 |
| 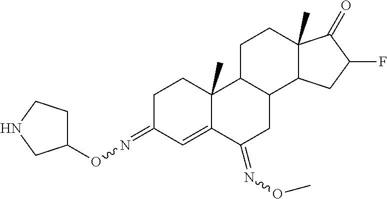 [SMILES: CON=C1CC2C(CC[C@]3(C)C(=O)C(F)CC23)[C@@]2(C)CCC(=NOC3CCNC3)C=C12] | 51 |
| 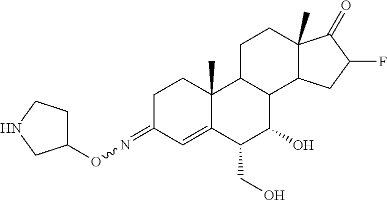 [SMILES: C[C@]12CCC(=NOC3CCNC3)C=C1[C@@H](CO)[C@@H](O)C1C2CC[C@]2(C)C(=O)C(F)CC12] | 52 |
| 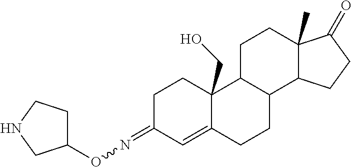 [SMILES: C[C@]12CCC3C(CCC4=CC(=NOC5CCNC5)CC[C@@]43CO)C1CCC2=O] | 53 |
| 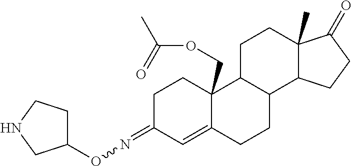 [SMILES: CC(=O)OC[C@]12CCC(=NOC3CCNC3)C=C1CCC1C2CC[C@]2(C)C(=O)CCC12] | 54 |
| 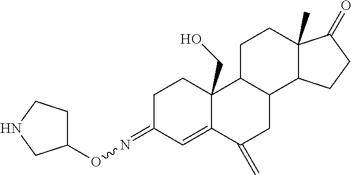 [SMILES: C=C1CC2C(CC[C@]3(C)C(=O)CCC23)[C@@]2(CO)CCC(=NOC3CCNC3)C=C12] | 55 |
| 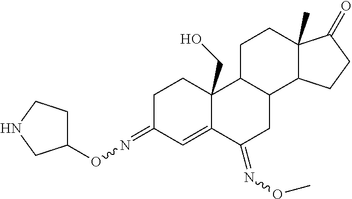 [SMILES: CON=C1CC2C(CC[C@]3(C)C(=O)CCC23)[C@@]2(CO)CCC(=NOC3CCNC3)C=C12] | 56 |
| 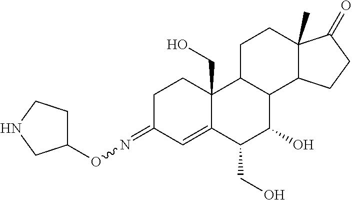 [SMILES: C[C@]12CCC3C(C1CCC2=O)[C@H](O)[C@H](CO)C1=CC(=NOC2CCNC2)CC[C@@]13CO] | 57 |
| 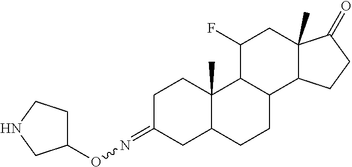 [SMILES: C[C@]12CCC(=NOC3CCNC3)CC1CCC1C2C(F)C[C@]2(C)C(=O)CCC12] | 58 |
| 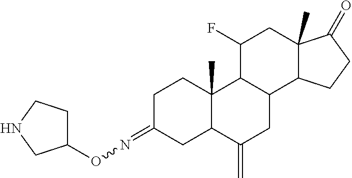 [SMILES: C=C1CC2C(C(F)C[C@]3(C)C(=O)CCC23)[C@@]2(C)CCC(=NOC3CCNC3)CC12] | 59 |
| 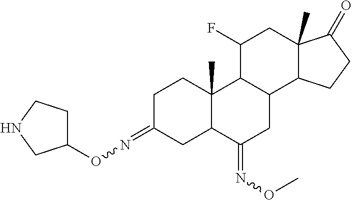 [SMILES: CON=C1CC2C(C(F)C[C@]3(C)C(=O)CCC23)[C@@]2(C)CCC(=NOC3CCNC3)CC12] | 60 |
| 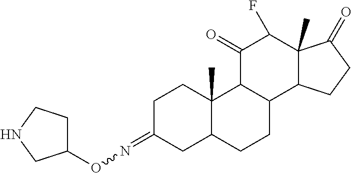 [SMILES: C[C@]12CCC(=NOC3CCNC3)CC1CCC1C2C(=O)C(F)[C@]2(C)C(=O)CCC12] | 61 |
| 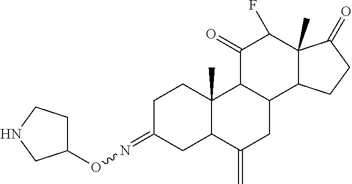 [SMILES: C=C1CC2C(C(=O)C(F)[C@]3(C)C(=O)CCC23)[C@@]2(C)CCC(=NOC3CCNC3)CC12] | 62 |
| 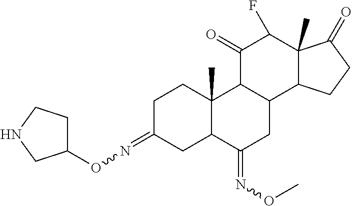 [SMILES: CON=C1CC2C(C(=O)C(F)[C@]3(C)C(=O)CCC23)[C@@]2(C)CCC(=NOC3CCNC3)CC12] | 63 |
| 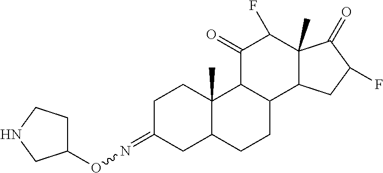 [SMILES: C[C@]12CCC(=NOC3CCNC3)CC1CCC1C2C(=O)C(F)[C@]2(C)C(=O)C(F)CC12] | 64 |
| 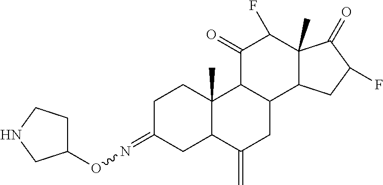 [SMILES: C=C1CC2C(C(=O)C(F)[C@]3(C)C(=O)C(F)CC23)[C@@]2(C)CCC(=NOC3CCNC3)CC12] | 65 |
| 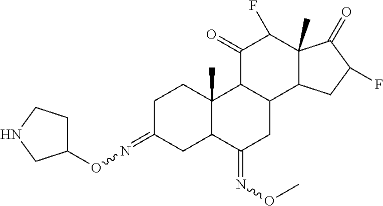 [SMILES: CON=C1CC2C(C(=O)C(F)[C@]3(C)C(=O)C(F)CC23)[C@@]2(C)CCC(=NOC3CCNC3)CC12] | 66 |
| 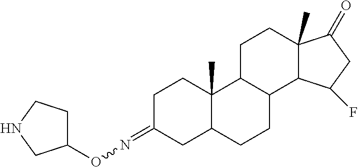 [SMILES: C[C@]12CCC(=NOC3CCNC3)CC1CCC1C2CC[C@]2(C)C(=O)CC(F)C12] | 67 |
| 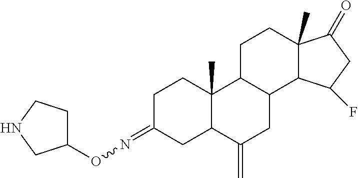 [SMILES: C=C1CC2C(CC[C@]3(C)C(=O)CC(F)C23)[C@@]2(C)CCC(=NOC3CCNC3)CC12] | 68 |
| 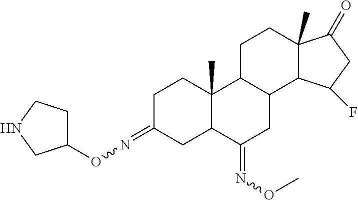 [SMILES: CON=C1CC2C(CC[C@]3(C)C(=O)CC(F)C23)[C@@]2(C)CCC(=NOC3CCNC3)CC12] | 69 |
| 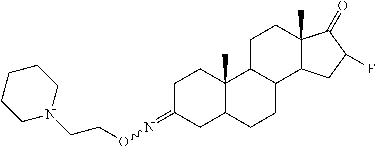 [SMILES: C[C@]12CCC(=NOCCN3CCCCC3)CC1CCC1C2CC[C@]2(C)C(=O)C(F)CC12] | 70 |
| 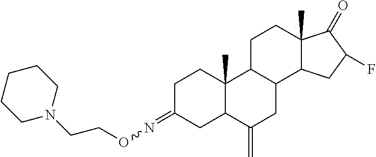 [SMILES: C=C1CC2C(CC[C@]3(C)C(=O)C(F)CC23)[C@@]2(C)CCC(=NOCCN3CCCCC3)CC12] | 71 |
| 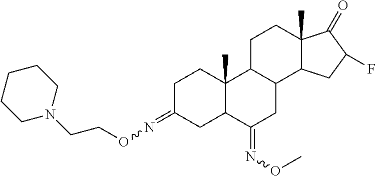 [SMILES: CON=C1CC2C(CC[C@]3(C)C(=O)C(F)CC23)[C@@]2(C)CCC(=NOCCN3CCCCC3)CC12] | 72 |
| 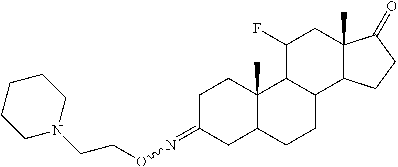 [SMILES: C[C@]12CCC(=NOCCN3CCCCC3)CC1CCC1C2C(F)C[C@]2(C)C(=O)CCC12] | 73 |
| 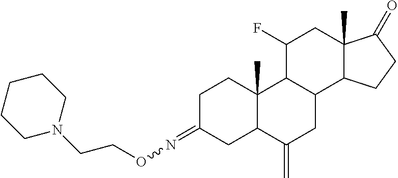 [SMILES: C=C1CC2C(C(F)C[C@]3(C)C(=O)CCC23)[C@@]2(C)CCC(=NOCCN3CCCCC3)CC12] | 74 |
| 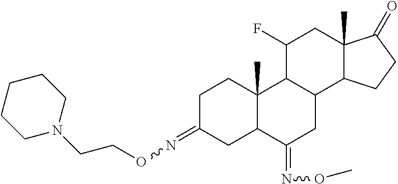 [SMILES: CON=C1CC2C(C(F)C[C@]3(C)C(=O)CCC23)[C@@]2(C)CCC(=NOCCN3CCCCC3)CC12] | 75 |
| 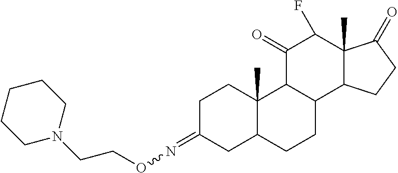 [SMILES: C[C@]12CCC(=NOCCN3CCCCC3)CC1CCC1C2C(=O)C(F)[C@]2(C)C(=O)CCC12] | 76 |
| 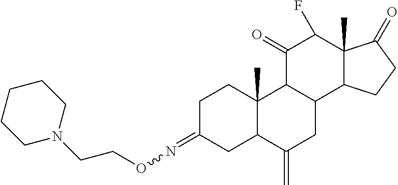 [SMILES: C=C1CC2C(C(=O)C(F)[C@]3(C)C(=O)CCC23)[C@@]2(C)CCC(=NOCCN3CCCCC3)CC12] | 77 |
| 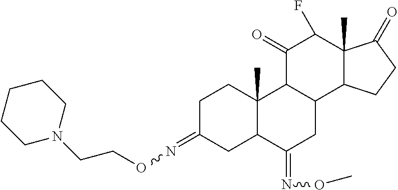 [SMILES: CON=C1CC2C(C(=O)C(F)[C@]3(C)C(=O)CCC23)[C@@]2(C)CCC(=NOCCN3CCCCC3)CC12] | 78 |
| 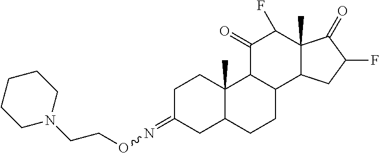 [SMILES: C[C@]12CCC(=NOCCN3CCCCC3)CC1CCC1C2C(=O)C(F)[C@]2(C)C(=O)C(F)CC12] | 79 |
| 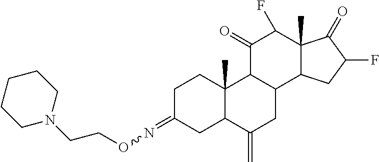 [SMILES: C=C1CC2C(C(=O)C(F)[C@]3(C)C(=O)C(F)CC23)[C@@]2(C)CCC(=NOCCN3CCCCC3)CC12] | 80 |
| 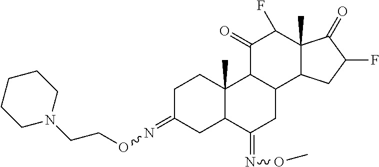 [SMILES: CON=C1CC2C(C(=O)C(F)[C@]3(C)C(=O)C(F)CC23)[C@@]2(C)CCC(=NOCCN3CCCCC3)CC12] | 81 |
| 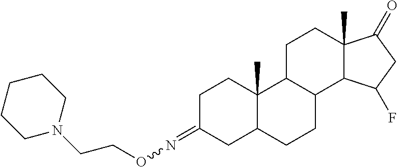 [SMILES: C[C@]12CCC(=NOCCN3CCCCC3)CC1CCC1C2CC[C@]2(C)C(=O)CC(F)C12] | 82 |
| 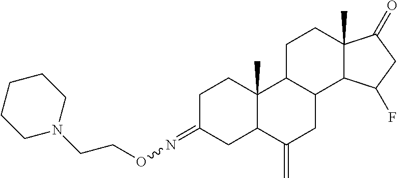 [SMILES: C=C1CC2C(CC[C@]3(C)C(=O)CC(F)C23)[C@@]2(C)CCC(=NOCCN3CCCCC3)CC12] | 83 |
| 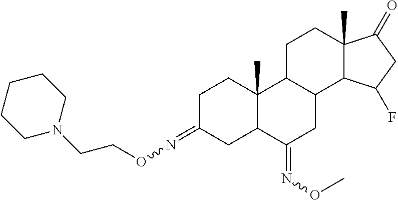 [SMILES: CON=C1CC2C(CC[C@]3(C)C(=O)CC(F)C23)[C@@]2(C)CCC(=NOCCN3CCCCC3)CC12] | 84 |
| 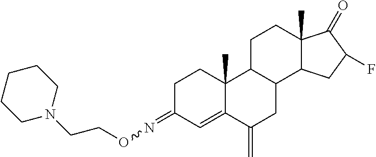 [SMILES: C=C1CC2C(CC[C@]3(C)C(=O)C(F)CC23)[C@@]2(C)CCC(=NOCCN3CCCCC3)C=C12] | 85 |
| 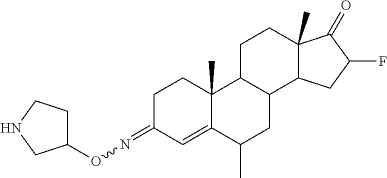 [SMILES: CC1CC2C(CC[C@]3(C)C(=O)C(F)CC23)[C@@]2(C)CCC(=NOC3CCNC3)C=C12] | 86 |

In a separate embodiment, U and Y together with the carbon to which they are attached to form C═O. In one embodiment, A is halogen and X is H. Examples of the compounds include, but are not limited to, Compound Nos. 61-66 and 76-81 as shown in Table 1 supra.

In another embodiment, both of U and Y are H. In one embodiment, A is H, and X is F. Examples of the compounds include, but are not limited to, Compound Nos. 67-69 and 82-84 as shown in Table 1 supra.

In yet another embodiment, one of U and Y is H, and the other is halogen. In one instance, both A and X are H (see Compound Nos. 58-60 and 73-75 provided in Table 1 supra.).

In a particular embodiment, the invention provides compounds of Formula (I) as follows:

[symbol] represents a single bond;

Both of A and X are H;

Both of U and Y are H;

R¹¹ and R¹² together with the carbon atom to which they are attached form C═O;

R¹ is amino(C₁-C₆)alkyl, a 4- to 6-membered heterocyclic ring (e.g., aminoethyl or pyrrolidinyl), or (C₁-C₆)alkyl substituted by a 4- to 6-membered heterocyclic ring;

R¹³ is unsubstituted (C₁-C₆)alkyl, or (C₁-C₆)alkyl that is substituted by hydroxyl or —OC(O)—(C₁-C₆)alkyl;

both of R² and R³ are H; or one of R² and R³ is H, the other is an optionally substituted (C₁-C₆)alkyl or an optionally substituted aryl;

R⁴ is H or —OH;

R⁵ and R⁶ are both H; or one of R⁵ and R⁶ is H, the other is (C₁-C₆)alkyl optionally substituted by hydroxyl or (C₁-C₄)alkoxy; or R⁵ and R⁶ together with the carbon to which they are attached form C═O, C═C(R¹⁶)₂, or C═N—OR²⁰;

one of R⁹ and R¹⁰ is H, and the other is H or halogen (e.g., F);

R⁷ and R⁸ are both H; or one of R⁷ and R⁸ is H, the other is —OH; or R⁷ and R⁸ together with the carbon to which they are attached form C═O, C═C(R¹⁶)₂, or C═N—OR²⁰;

R¹⁶ for each occurrence is the same and is H or F; and

R²⁰ is (C₁-C₆)alkyl.

Examples of the compounds include, but are not limited to, Compound Nos. 1-48, and 70-72 as shown in Table 1 supra.

In another embodiment, the invention provides compounds of Formula (I) as follows:

[symbol] represents a single bond;

One of A and X is H, and the other is halogen (e.g., F);

One of U and Y is H, or the other is H or halogen (e.g., F); or U and Y together with the carbon to which they are attached to form C═O;

R¹¹ and R¹² together with the carbon atom to which they are attached form C═O;

R¹ is amino(C₁-C₆)alkyl, 4- to 6-membered heterocyclic ring (e.g., aminoethyl or pyrrolidinyl), or (C₁-C₆)alkyl substituted by a 4- to 6-membered heterocyclic ring;

R¹³ is unsubstituted (C₁-C₆)alkyl, or (C₁-C₆)alkyl that is substituted by hydroxyl or —OC(O)—(C₁-C₆)alkyl;

both of R² and R³ are H; or one of R² and R³ is H, the other is an optionally substituted (C₁-C₆)alkyl or an optionally substituted aryl;

R⁴ is H or —OH;

R⁵ and R⁶ are both H; or one of R⁵ and R⁶ is H, the other is (C₁-C₆)alkyl optionally substituted by hydroxyl or (C₁-C₄)alkoxy; or R⁵ and R⁶ together with the carbon to which they are attached form C═O, C═C(R¹⁶)₂, or C═N—OR²⁰;

one of R⁹ and R¹⁰ is H, and the other is H or halogen (e.g., F);

R⁷ and R⁸ are both H; or one of R⁷ and R⁸ is H, the other is —OH; or R⁷ and R⁸ together with the carbon to which they are attached form C═O, C═C(R¹⁶)₂, or C═N—OR²⁰;

R¹⁶ for each occurrence is the same and is H or F; and

R²⁰ is (C₁-C₆)alkyl.

Examples of the compounds include, but are not limited to, Compound Nos. 58-69 and 73-84 as shown in Table 1 supra.

In another embodiment, the invention provides compounds of Formula (I) as follows:

[symbol] represents a double bond;

Both of A and X are H;

Both of U and Y are H;

R¹¹ and R¹² together with the carbon atom to which they are attached form C═O;

R¹ is amino(C₁-C₆)alkyl, a 4- to 6-membered heterocyclic ring, or (C₁-C₆)alkyl substituted by a 4- to 6-membered heterocyclic ring (e.g., aminoethyl, pyrrolidinyl, piperidinylethyl);

R¹³ is an optionally substituted (C₁-C₆)alkyl (e.g., —CH₃, —CH₂OC(O)CH₃ and —CH₂OH);

One of R² and R³ is absent, and the other is H;

R⁴ is H;

One of R⁵ and R⁶ is H, and the other is H or (C₁-C₆)alkyl optionally substituted by hydroxyl; or R⁵ and R⁶ together with the carbon to which they are attached form C═O, C═C(R¹⁶)₂, or C═N—OR²⁰;

One of R⁷ and R⁸ is H, and the other is H or —OH;

One of R⁹ and R¹⁰ is H, and the other is H or halogen;

R¹⁶ for each occurrence is the same and is H or F;

R²⁰ is (C₁-C₆)alkyl.

Examples of the compounds include, but are not limited to, Compound Nos. 49-57 and 85-86 as shown in Table 1 supra.

In another aspect, the invention provides a compound of Formula (Ia):

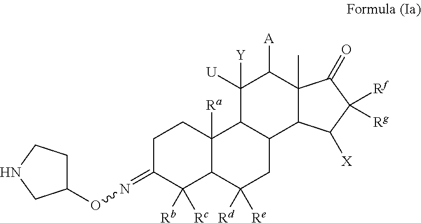

wherein:

A and X, for each occurrence, independently is H or halogen;

One of U and Y is H, and the other is H, or halogen; or U and Y together with the carbon to which they are attached form C═O;

R^{a} is H or (C₁-C₆)alkyl that is further optionally substituted by hydroxyl or —OC(O)—(C₁-C₆)alkyl;

One of R^{b} and R^{c} is H or halogen, and the other is H, halogen, optionally substituted aryl, optionally substituted heteroaryl, alkyl, or OH;

One of R^{d} and R^{e} is H or OH, and the other is H or —OH; or R^{d} and R^{e} together with the carbon to which they are attached form C═O, C═C(R^{j})₂, or C═N—OR^{k}; One of R^{f} and R^{g} is H, and the other is H or halogen;

R^{j} is the same or different on each occurrence and is H or halogen;

R^{k} is amino(C₁-C₆)alkyl or (C₁-C₆)alkyl;

or a tautomer, stereoisomer, Z and/or E isomer, optical isomer, N-oxide, hydrate, solvate, polymorph, pharmaceutically acceptable ester, amide, salt, prodrug, and isotopic derivative thereof;

provided that

when R^{a} is methyl and R^{f} and R^{g} are both H, one of R^{b} and R^{c} must be an optionally substituted aryl or optionally substituted heteroaryl.

In one embodiment, both of A and X are H. In another embodiment, both of U and Y are H.

In one embodiment, the invention provides compounds of Formula (Ia) with both of R^{f} and R^{g} being H.

In an embodiment, R^{a} is (C₁-C₆)alkyl (e.g., methyl). In another embodiment, R^{a} is (C₁-C₆)alkyl substituted by hydroxyl or —OC(O)—(C₁-C₆)alkyl. For instance, R^{a} can be —CH₂OH or —CH₂OC(O)CH₃.

One embodiment provides that R^{d} and R^{e} are both H. In another embodiment, R^{d} and R^{e} together with the carbon to which they are attached form C═O. In another embodiment provides that R^{d} and R^{e} together with the carbon to which they are attached form C═C(R^{j})₂. R^{j}, in certain circumstances, is the same on each occurrence and is H or halogen (e.g., F). Still further, R^{d} and R^{e} together with the carbon to which they are attached form C═N—OR^{k}. R^{K} can be, for example, (C₁-C₆)alkyl (e.g., methyl).

In an embodiment of the invention, one of R^{b} and R^{c} is H, and the other is an optionally substituted aryl (e.g., phenyl). In another embodiment, both of R^{b} and R^{c} are H.

In one embodiment, one of R^{f} and R^{g} is H, and the other is halogen (such as, F). In another embodiment, both of R^{f} and R^{g} are H.

In a separate embodiment, U and Y together with the carbon to which they are attached to form C═O. In one embodiment, A is halogen and X is H.

In another embodiment, both of U and Y are H. In one embodiment, A is H, and X is F.

In yet another embodiment, one of U and Y is H, and the other is halogen. In one instance, both of A and X are H.

Compounds of Formula (Ia), in accordance with the invention, include those described in Table 2 as follows:

TABLE 2

| | |
|---|---|
| 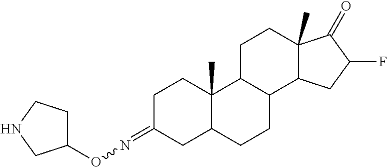 [SMILES: C[C@]12CCC(=NOC3CCNC3)CC1CCC1C2CC[C@]2(C)C(=O)C(F)CC12] | 2 |
| 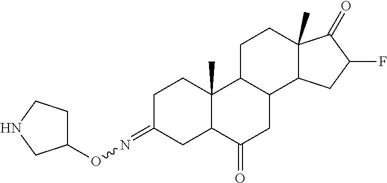 [SMILES: C[C@]12CCC(=NOC3CCNC3)CC1C(=O)CC1C2CC[C@]2(C)C(=O)C(F)CC12] | 4 |
| 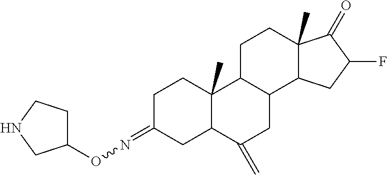 [SMILES: C=C1CC2C(CC[C@]3(C)C(=O)C(F)CC23)[C@@]2(C)CCC(=NOC3CCNC3)CC12] | 6 |
| 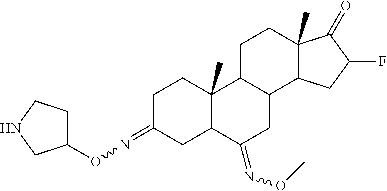 [SMILES: CON=C1CC2C(CC[C@]3(C)C(=O)C(F)CC23)[C@@]2(C)CCC(=NOC3CCNC3)CC12] | 8 |
| 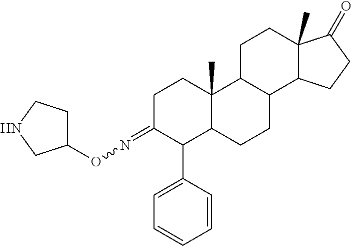 [SMILES: C[C@]12CCC(=NOC3CCNC3)C(c3ccccc3)C1CCC1C2CC[C@]2(C)C(=O)CCC12] | 21 |
| 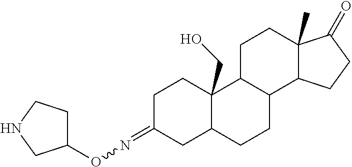 [SMILES: C[C@]12CCC3C(CCC4CC(=NOC5CCNC5)CC[C@@]43CO)C1CCC2=O] | 25 |
| 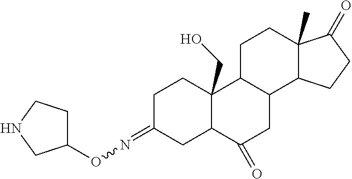 [SMILES: C[C@]12CCC3C(CC(=O)C4CC(=NOC5CCNC5)CC[C@@]43CO)C1CCC2=O] | 28 |
| 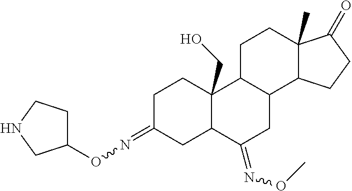 [SMILES: CON=C1CC2C(CC[C@]3(C)C(=O)CCC23)[C@@]2(CO)CCC(=NOC3CCNC3)CC12] | 30 |
| 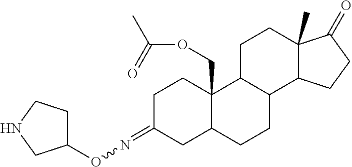 [SMILES: CC(=O)OC[C@]12CCC(=NOC3CCNC3)CC1CCC1C2CC[C@]2(C)C(=O)CCC12] | 48 |
| 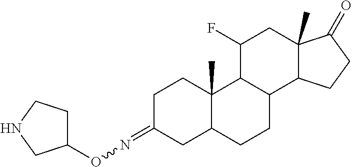 [SMILES: C[C@]12CCC(=NOC3CCNC3)CC1CCC1C2C(F)C[C@]2(C)C(=O)CCC12] | 58 |
| 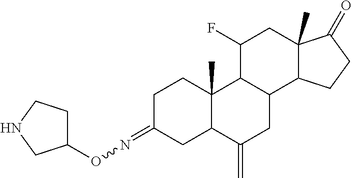 [SMILES: C=C1CC2C(C(F)C[C@]3(C)C(=O)CCC23)[C@@]2(C)CCC(=NOC3CCNC3)CC12] | 59 |
| 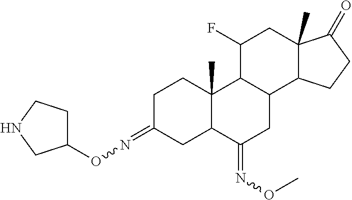 [SMILES: CON=C1CC2C(C(F)C[C@]3(C)C(=O)CCC23)[C@@]2(C)CCC(=NOC3CCNC3)CC12] | 60 |
| 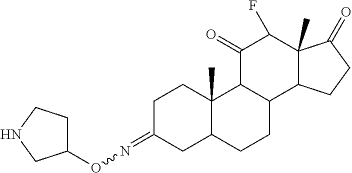 [SMILES: C[C@]12CCC(=NOC3CCNC3)CC1CCC1C2C(=O)C(F)[C@]2(C)C(=O)CCC12] | 61 |
| 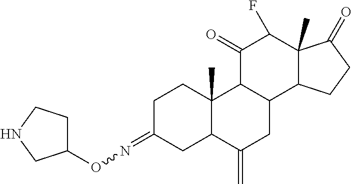 [SMILES: C=C1CC2C(C(=O)C(F)[C@]3(C)C(=O)CCC23)[C@@]2(C)CCC(=NOC3CCNC3)CC12] | 62 |
| 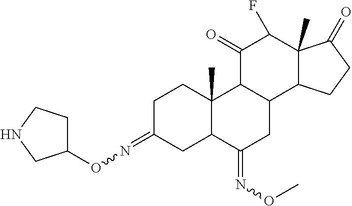 [SMILES: CON=C1CC2C(C(=O)C(F)[C@]3(C)C(=O)CCC23)[C@@]2(C)CCC(=NOC3CCNC3)CC12] | 63 |
| 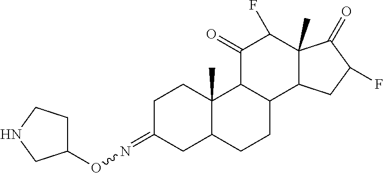 [SMILES: C[C@]12CCC(=NOC3CCNC3)CC1CCC1C2C(=O)C(F)[C@]2(C)C(=O)C(F)CC12] | 64 |
| 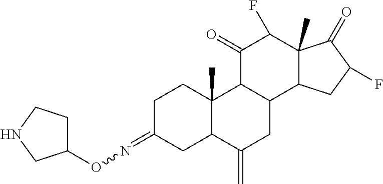 [SMILES: C=C1CC2C(C(=O)C(F)[C@]3(C)C(=O)C(F)CC23)[C@@]2(C)CCC(=NOC3CCNC3)CC12] | 65 |
| 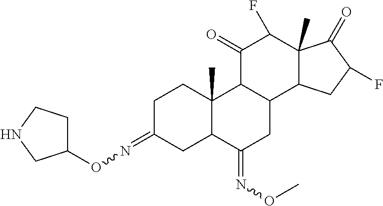 [SMILES: CON=C1CC2C(C(=O)C(F)[C@]3(C)C(=O)C(F)CC23)[C@@]2(C)CCC(=NOC3CCNC3)CC12] | 66 |
| 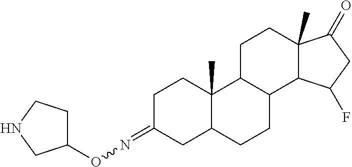 [SMILES: C[C@]12CCC(=NOC3CCNC3)CC1CCC1C2CC[C@]2(C)C(=O)CC(F)C12] | 67 |
| 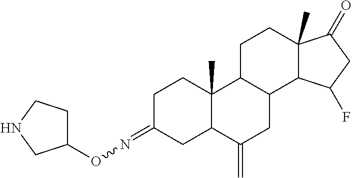 [SMILES: C=C1CC2C(CC[C@]3(C)C(=O)CC(F)C23)[C@@]2(C)CCC(=NOC3CCNC3)CC12] | 68 |
| 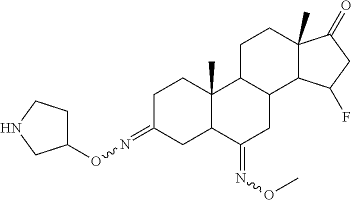 [SMILES: CON=C1CC2C(CC[C@]3(C)C(=O)CC(F)C23)[C@@]2(C)CCC(=NOC3CCNC3)CC12] | 69 |

In another aspect, the invention provides compounds of Formula (II):

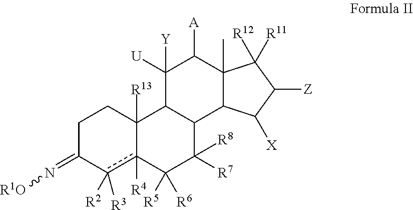

wherein:

[symbol] represents a single or double bond;

A and X, for each occurrence, independently is H or halogen;

One of U and Y is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸;

or U and Y together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;

Z is halogen;

R¹ is amino(C₁-C₆)alkyl, or a 4- to 6-membered heterocyclic ring, or (C₁-C₆)alkyl substituted by a 4- to 6-membered heterocyclic ring (e.g., aminoethyl, pyrrolidinyl, or piperidinylethyl);

One of R² and R³ is H, halogen, optionally substituted (C₁-C₆)alkyl, OH, or absent, and the other is H, halogen, optionally substituted aryl, optionally substituted heteroaryl, (C₁-C₆)alkyl, OH, or absent;

Or R² and R³ together with the carbon to which they are attached form cycloalkyl;

R⁴ is H, —OH, or absent;

One of R⁵ and R⁶ is H, —OH, or halogen, and the other is H; —OR¹⁴; alkyl optionally substituted with hydroxyl or alkoxy; —C(O)NH₂; —C(O)—O-alkyl; —NHR¹⁵; or alkynyl;

or R⁵ and R⁶ together with the carbon to which they are attached form C═O, C═C(R¹⁶)₂, or C═N—OR²⁰;

One of R⁷ and R⁸ is H, or halogen, and the other is H; —OR¹⁴; alkyl optionally substituted with hydroxyl or alkoxy; —C(O)—NH₂; —C(O)—O-alkyl; —NHR¹⁵; alkynyl; or R⁷ and R⁸ together with the carbon to which they are attached form C═O or C═C(R¹⁶)₂;

R¹¹ and R¹² together with the carbon atom to which they are attached form C═O;

R¹³ is an optionally substituted (C₁-C₆)alkyl (e.g., —CH₃, —CH₂OC(O)CH₃ and —CH₂OH);

R¹⁴ is H or alkyl;

R¹⁵ is H or formyl;

R¹⁶, for each occurrence, independently, is H or F;

R¹⁷ and R¹⁸, for each occurrence, independently is H, alkyl, alkenyl, alkynyl, cycloalkyl, aryl, aminocarbonyl, alkoxycarbonyl, amino, or halogen; wherein R¹⁷ and R¹⁸ independently are further optionally substituted with one or more moieties selected from the group of alkyl, alkenyl, alkynyl, amino, alkoxy, and cycloalkyl,

R²⁰ is (C₁-C₆)alkyl.

In certain embodiments, Z is fluorine. Specific embodiments of such compounds of Formula (II) include, but are not limited to, Compound Nos. 1, 3, 5, 7, 49-52, 70-72, 79-81, 85 and 86 20, 49-52, 64-66, 70-72, 79-81, and 85-86 as shown in Table 1 supra.

In certain embodiments of the compounds of Formula II, the invention provides compounds of Formula (IIa):

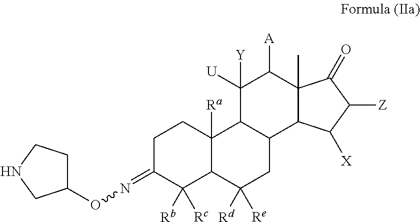

wherein:

A and X, for each occurrence, independently is H or halogen;

One of U and Y is H, and the other is H or halogen; or U and Y together with the carbon to which they are attached form C═O;

Z is halogen;

R^{a} is H, or (C₁-C₆)alkyl that is further optionally substituted by hydroxyl or —OC(O)—(C₁-C₆)alkyl;

One of R^{b} and R^{c} is H, or halogen, and the other is H, halogen, optionally substituted aryl, optionally substituted heteroaryl, alkyl, or OH;

One of R^{d} and R^{e} is H or OH, and the other is H or —OH; or R^{d} and R^{e} together with the carbon to which they are attached form C═O, C═C(R)₂, or C═N—OR^{k};

R^{j} is the same or different on each occurrence and is H or halogen;

R^{k} is amino(C₁-C₆)alkyl or (C₁-C₆)alkyl;

or a tautomer, stereoisomer, Z and/or E isomer, optical isomer, N-oxide, hydrate, solvate, polymorph, pharmaceutically acceptable ester, amide, salt, prodrug, and isotopic derivative thereof;

provided that

when R^{a} is methyl and R^{f} and R^{g} are both hydrogen, one of R^{b} and R^{c} must be an optionally substituted aryl or optionally substituted heteroaryl.

In certain embodiments, Z is fluorine. Examples of the such compounds of Formula (IIa) include, but are not limited to, Compound Nos. 2, 4, 6, 8, 64, 65 and 66 as shown in Table 2 supra.

As noted above, certain embodiments of the invention provide compounds of Formulae (II) and (IIa) in which Z is halogen. In further embodiments, the halogen is fluorine, providing 16-fluoro compounds.

It is believed that certain fluorinated compounds of the invention offer good pharmacological properties, good water solubility, and/or good specificity toward hormonal receptors. In certain instances, the fluorinated compounds of the invention have good metabolic stability, which is one of the key factors in determining the bioavailability of a compound. In other instances, certain fluorinated compounds of the invention induce a reduction in the activity of cytochrome p450 CYP1A1 and CYP1B1, enzymes typically linked to the metabolism of steroids.

It has been observed that certain fluorinated compounds of the invention have a good solubility in water.

It is believed that certain fluorinated compounds of the invention have relatively long half-lives, which may contribute to a reduced dosing regimen. It is also believed that certain fluorinated compounds of the invention have good compound lipophilicity, which in turn offers good binding affinity to a target protein. In certain instances, the fluorinated compounds of the invention exhibit highly reduced hormone binding (e.g., reduced mineralcorticoid activity), reduced glucocorticoid activity, and/or reduced estrogen receptor binding activity. In particular, it is believed that certain fluorinated compounds of the invention are devoid of estrogenic and androgenic activities.

In certain embodiments, it is believed that a compound of the invention is capable of preventing or treating a neoplasia (e.g., a membrane androgen positive solid tumor or hematological malignancy) in a subject. The compounds of the invention are believed to act as Na⁺K⁺ ATPase inhibitors, which inhibit ligand binding to a membrane androgen receptor, thereby preventing or treating the neoplasia.

In certain instances, it is believed that a compound of the invention binds a Na⁺K⁺ ATPase and inhibits Na⁺K⁺ ATPase activity. A compound of the invention may bind to a membrane androgen receptor and competitively inhibit ligand binding to the receptor. In other instances, it is believed that a compound of the invention induces cell death in a neoplastic cell of the neoplasia. In some embodiments, the neoplasia is a lung cancer, prostate cancer, breast cancer, CNS cancer, kidney cancer, or colon cancer.

A compound of the invention may also be used in treating or preventing prostate cancer in a subject. The compound may bind and inhibit a Na⁺K⁺ ATPase, and also inhibit ligand binding to the membrane androgen receptor on a prostate cancer cell. In one instance, a compound of the invention may induce cell death (e.g., apoptosis) in a cell of the prostate cancer. In another instance, a compound of the invention may bind the membrane androgen receptor.

### Uses of the Compounds of the Invention

The invention also provides methods for treating a subject for a neoplasia by administering to the subject an effective amount of a compound of the invention. In certain embodiments, the subject is a mammal, in particular a human.

In accordance with the invention, compounds are administered in combination with a pharmaceutically diluent or acceptable carrier. In one embodiment, the compound can be administered using a pharmaceutically acceptable formulation. In advantageous embodiments, the pharmaceutically-acceptable carrier provides sustained delivery of the compound to a subject for at least four weeks after administration to the subject.

In certain embodiments, the compound is administered orally. In other embodiments, the compound is administered intravenously. In yet other embodiments, the compound is administered topically. In still other embodiments, the compound is administered topically or parenterally.

Although dosages may vary depending on the particular indication, route of administration and subject, the compounds are typically administered at a concentration of about 0.1 g to 20 milligram per kilogram of body weight per day (mg/kg/day) (e.g., 0.1 μg/kg to 2 mg/kg, 0.3-3 μg/kg, 0.18-0.54 mg/kg). In other embodiments, the amount varies from about 0.1 mg/kg/day to about 100 mg/kg/day. In still other embodiments, the amount varies from about 0.001 g to about 100 μg/kg (e.g., of body weight).

Determination of a therapeutically effective amount or a prophylactically effective amount of a compound described herein can readily be made by one skilled in the art. The dosages may be varied depending upon the requirements of the patient, the severity of the condition being treated and the particular compound being employed. In determining the therapeutically effective amount or dose, and the prophylactically effective amount or dose, a number of factors are considered, including, but not limited to: the specific hyperplastic/neoplastic cell involved; pharmacodynamic characteristics of the particular agent and its mode and route of administration; the desired time course of treatment; the species of mammal; its size, age, and general health; the specific disease involved; the degree of or involvement or the severity of the disease; the response of the individual subject; the particular compound administered; the mode of administration; the bioavailability characteristics of the preparation administered; the dose regimen selected; the kind of concurrent treatment (i.e., the interaction of the compounds of the invention with other co-administered therapeutics); and other relevant circumstances. U.S. Pat. No. 5,427,916, for example, describes method for predicting the effectiveness of antineoplastic therapy in individual patients, and illustrates certain methods which can be used in conjunction with the treatment protocols of the instant invention.

Treatment can be initiated with smaller dosages which are less than the optimum dose of the compound. Thereafter, the dosage should be increased by small increments until the optimum effect under the circumstances is reached. For convenience, the total daily dosage may be divided and administered in portions during the day if desired. A therapeutically effective amount and a prophylactically effective amount of a compound of the invention is expected to vary from about 0.1 g to 20 milligram per kilogram of body weight per day (mg/kg/day) (e.g., 0.1 μg/kg to 2 mg/kg, 0.3-3 μg/kg, 0.18-0.54 mg/kg). In other embodiments, the amount varies from about 0.1 mg/kg/day to about 100 mg/kg/day. In still other embodiments, the amount varies from about 0.001 g to about 100 μg/kg (e.g., of body weight).

Compounds which are determined to be effective for the prevention or treatment of neoplasias in animals, e.g., dogs, rodents, may also be useful in treatment of neoplasias in humans. Those skilled in the art of treating neoplasias in humans will know, based upon the data obtained in animal studies, the dosage and route of administration of the compound to humans. In general, the dosage and route of administration in humans is expected to be similar to that in animals.

The identification of those patients who are in need of prophylactic treatment for hyperplastic/neoplastic disease states is well within the ability and knowledge of one skilled in the art. Certain of the methods for identification of patients who are at risk of developing neoplastic disease states which can be treated by the subject method are appreciated in the medical arts, such as family history of the development of a particular disease state and the presence of risk factors associated with the development of that disease state in the subject patient. A clinician skilled in the art can readily identify such candidate patients, by the use of, for example, clinical tests, physical examination and medical/family history.

Another aspect of the invention comprises obtaining the compound of the invention.

### Neoplasia

The invention features methods for inhibiting the proliferation, growth, or viability of a neoplastic cell by contacting the cells with a compound of the invention. In general, the method includes a step of contacting a neoplastic cell with an effective amount of a compound of the invention. The present method can be performed on cells in culture, e.g., in vitro or ex vivo, or can be performed on cells present in an animal subject, e.g., as part of an in vivo therapeutic protocol. The therapeutic regimen can be carried out on a human or other subject.

The compounds of the invention or otherwise described herein can be tested initially in vitro for their inhibitory effects on the proliferation or survival of neoplastic cells. Examples of cell lines that can be used are lung cancer cell lines (e.g., H460, EKVX, A549), breast cancer cell lines (e.g., MCF7), CNS cancer cell lines (e.g., SF268, U251), colon cancer cell lines (e.g., HCT116, HCT15), prostate (e.g., PC3, DU145), ovarian cancer cell lines (e.g., IGROV1, OVCAR5, OVCAR3, NCI-ADRRES), pancreatic cancer cell lines (e.g., SU8686), renal cancer cell lines (e.g., CAKI), and melanoma cancer cell lines (e.g., LOXIMVI, SKMEL28, MB435). Alternatively, the antineoplastic activity of compounds of the invention can be tested in vivo using various animal models known in the art. For example, xenographs of human neoplastic cells or cell lines are injected into immunodeficient mice (e.g., nude or SCID) mice. Compounds of the invention are then administered to the mice and the growth and/or metastasis of the tumor is compared in mice treated with a compound of the invention relative to untreated control mice. Agents that reduce the growth or metastasis of a tumor or increase mice survival are identified as useful in the methods of the invention.

The methods discussed herein can be used to inhibit the proliferation of virtually any neoplastic cell. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

### Multidrug Resistant Neoplasias

It is believed that neoplasias that are resistant or refractory to anti-neoplastic therapies are likely to be susceptible to treatment with the compounds delineated herein (e.g., compounds 2, 4, 6, 8, 21, 25, 28, 30, and 48). Neoplasias that display resistance to a wide variety of chemotherapeutic agents are described as multidrug resistant. Multidrug resistant neoplasias are characterized by their ability to resist treatment with compounds having diverse structures and mechanisms of action. Multidrug resistance is generally related to alterations in a family of proteins known as ATP-binding cassette (ABC) transporters.

Multidrug resistant neoplasias often display increased expression of ATP-binding cassette (ABC) transporters, which function as ATP-dependent efflux pumps. These pumps actively transport a wide array of anti-cancer and cytotoxic drugs out of the cell. In mammals, the superfamily of ABC transporters includes P-glycoprotein (P-gp) transporters (MDR1 and MDR3 genes in human), the MRP subfamily, and bile salt export protein (ABCB 11; Cancer Res (1998) 58, 4160-4167), MDR-3 (Nature Rev Cancer (2002) 2, 48-58), lung resistance protein (LRP) and breast cancer resistant protein (BCRP). These proteins can recognize and efflux numerous substrates with unrelated chemical structures, including many chemotherapeutics. Other causes of multidrug resistance have been attributed to changes in topoisomerase II, protein kinase C and specific glutathione transferase enzymes.

In particular embodiments, compounds of the invention (e.g., compounds 2, 4, 6, 8, 21, 25, 28, 30, and 48) are particularly useful for neoplasias showing alterations in the activity or expression of MDR1, MDR2, or P-gP). In particular embodiments, the drug resistance of the tumor is mediated through the overexpression of P-gp. Numerous mechanisms can lead to overexpression of P-gp, including amplification of the MDR-1 gene (Anticancer Res (2002) 22, 2199-2203), increased transcription of the MDR-1 gene (J Clin Invest (1995) 95, 2205-2214; Cancer Lett (1999) 146, 195-199; Clin Cancer Res (1999) 5, 3445-3453; Anticancer Res (2002) 22, 2199-2203), by mutations in the MDR-1 gene (Cell (1988) 53, 519-529; Proc Natl Acad Sci USA (1991) 88, 7289-7293; Proc Natl Acad Sci USA (1992) 89, 4564-4568) and chromosomal rearrangements involving the MDR-1 gene (J Clin Invest (1997) 99, 1947-1957).

Therapeutic agents to which resistance is conferred via the action of P-gp include, but are not limited to: vinca alkaloids (e.g., vinblastine), the anthracyclines (e.g., adriamycin, doxorubicin), the epipodophyllotoxins (e.g., etoposide), taxanes (e.g., paclitaxel, docetaxel), antibiotics (e.g., actinomycin D and gramicidin D), antimicrotubule drugs (e.g., colchicine), protein synthesis inhibitors (e.g., puromycin), toxic peptides (e.g., valinomycin), topoisomerase Inhibitors (e.g., topotecan), DNA intercalators (e.g., ethidium bromide) and anti-mitotics. See WO 99/20791. The methods and pharmaceutical compositions of the present invention are useful for treating tumors resistant to any one or more of above-listed drugs.

In still other embodiments, the methods of the invention are useful for treating resistant or refractory neoplasias, where the resistance is conferred by an alteration in a topoisomerase (e.g., topoisomerase II), protein kinase C and specific glutathione transferase enzyme. Methods of the invention are also useful for the treatment of neoplasias showing resistance to taxanes (e.g., paclitaxel and docetaxel). Such resistance is typically mediated by alterations in tubulin. In other embodiments, compounds delineated herein are useful for treating neoplasias that are refectory to platinum-based chemotherapeutic agents, including carboplatin, cisplatin, oxaliplatin, iproplatin, tetraplatin, lobaplatin, DCP, PLD-147, JM118, JM216, JM335, and satraplatin. Such platinum-based chemotherapeutic agents also include the platinum complexes disclosed in EP 0147926, U.S. Pat. No. 5,072,011, U.S. Pat. Nos. 5,244,919, 5,519,155, 6,503,943 (LA-12/PLD-147), 6,350,737, and WO 01/064696 (DCP).

In sum, the methods and pharmaceutical compositions of the invention are generally useful for treating resistant and/or refractory neoplasias to any one or more of drugs known in the art or described herein. In particular embodiments, the methods and compositions of the invention are useful for the treatment of patients having end-stage disease, which includes patients for whom no effective therapeutic regimen exists or patients identified as having less than about 3, 6, 9 or 12 months to live.

### Combination Therapies

In certain embodiments, the compounds of the invention are administered in combination with any other standard anti-neoplasia therapy or conventional chemotherapeutic agent, such as an alkylating agent; such methods are known to the skilled artisan and described in Remington's Pharmaceutical Sciences by E. W. Martin. For example, if desired, agents of the invention (e.g., compounds 2, 4, 6, 8, 21, 25, 28, 30, and 48) are administered in combination with any conventional anti-neoplastic therapy, including but not limited to, surgery, radiation therapy, or chemotherapy. Conventional chemotherapeutic agents include, but are not limited to, abiraterone, alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine. In particular embodiments, a combination of the invention comprises any one or more of the following: vinca alkaloids (e.g., vinblastine), taxanes (e.g., paclitaxel, docetaxel), epothilones (e.g., ixabepilone), antifolates (e.g., Methotrexate), purine analogs (e.g., fludarabine), pyrimidine analogs (e.g., gemcitabine), DNA intercalators (e.g., ethidium bromide), topoisomerase Inhibitors (e.g., topotecan), alkylating agents (e.g., carmustine, bendamustine), platinum-based agents (e.g., cisplatin, oxaliplatin), receptor antagonists (e.g., atrasentan), hormone agents (e.g. anti-androgens, aromatase inhibitors), anthracyclines (e.g., adriamycin, doxorubicin), epipodophyllotoxins (e.g., etoposide), antibiotics (e.g., actinomycin D and gramicidin D), antimicrotubule drugs (e.g., colchicine), protein synthesis inhibitors (e.g., puromycin), toxic peptides (e.g., valinomycin), enzyme inhibitors (e.g. CDK inhibitors) and anti-mitotics.

The compounds can be administered concurrently or sequentially with chemotherapy. Alternatively, they can be administered using specific administration regimens. For example, to treat a prostate tumor xenograft in mice, the compounds of the invention could be administered intraperitoneally (ip) daily for 5 consecutive days followed by 2 days rest for 4 weekly cycles in total [QdX5; 2)ₓ₄], whereas a chemotherapeutic drug (e.g. a taxane) can be administered (ip) every 4 days for a total of 3 injections (Q4Dx3). Alternatively, in colon cancer xenograft models in mice, the compounds of the invention could be administered via implanted, pre-filled pumps (e.g Alzet pumps) continuously delivering the compound at a pre-determined rate, whereas the chemotherapeutic agent (e.g. irinotecan) can be administered (ip) via a Q4Dx3 scheme.

### Pharmaceutical Compositions

The invention also provides pharmaceutical compositions for the treatment of a neoplasia, comprising an effective amount a compound of the invention and a pharmaceutically acceptable carrier. In particular embodiments, compositions of the invention comprise a compound described herein in combination with a conventional chemotherapeutic agent. In still other embodiments, such compositions are labeled for the treatment of cancer. In a further embodiment, the effective amount is effective to reduce the growth, proliferation, or survival of a neoplastic cell or to otherwise treat or prevent a neoplasia in a subject, as described herein.

In an embodiment, the compound is administered to the subject using a pharmaceutically-acceptable formulation. In certain embodiments, these pharmaceutical compositions are suitable for oral or parenteral administration to a subject. In still other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. In certain embodiments, the subject is a mammal, e.g., a primate, e.g., a human.

The methods of the invention further include administering to a subject a therapeutically effective amount of a compound in combination with a pharmaceutically acceptable excipient. The phrase “pharmaceutically acceptable” refers to those compounds of the invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable excipient” includes pharmaceutically-acceptable material, composition or vehicle, such as a liquid or solid filler, diluent, carrier, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a compound(s) with the carrier and, optionally, one or more accessory ingredients. In general, the formulations are prepared by uniformly and intimately bringing into association a compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants, such as wetting agents, emulsifying and suspending agents, sweetening, flavoring, coloring, perfuming and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound(s) include powders, sprays, ointments, pastes, creams, lotions, gels, solutions, patches and inhalants. The active compound(s) may be mixed under sterile conditions with a pharmaceutically-acceptable carrier, and with any preservatives, buffers, or propellants which may be required.

The ointments, pastes, creams and gels may contain, in addition to compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound(s), excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids, such as glycine, buffers, salts, sugars or sugar alcohols. Aerosols generally are prepared from isotonic solutions.

Transdermal patches have the added advantage of providing controlled delivery of a compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound(s) in biodegradable polymers, such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. An exemplary dose range is from about 0.1 g to 20 milligram per kilogram of body weight per day (mg/kg/day) (e.g., 0.1 μg/kg to 2 mg/kg, 0.3-3 μg/kg, 0.18-0.54 mg/kg). In other embodiments, the amount varies from about 0.1 mg/kg/day to about 100 mg/kg/day. In still other embodiments, the amount varies from about 0.001 g to about 100 μg/kg (e.g., of body weight). Ranges intermediate to the above-recited values are also intended to be part of the invention.

### Kits

The invention provides kits for the treatment or prevention of neoplasia. In one embodiment, the kit includes a therapeutic or prophylactic composition containing an effective amount of a compound of the invention in unit dosage form. In some embodiments, a compound of the invention is provided in combination with a conventional chemotherapeutic agent. In other embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, a compound of the invention is provided together with instructions for administering the compound to a subject having or at risk of developing neoplasia. The instructions will generally include information about the use of the composition for the treatment or prevention of neoplasia. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXEMPLIFICATION OF THE INVENTION

The following examples are put forth so as to provide those of ordinary skill in the art with a complete disclosure and description of how to make and use the assay, screening, and therapeutic methods of the invention, and are not intended to limit the scope of what the inventors regard as their invention.

### I. CHEMICAL EXAMPLES

### Synthesis and Methods of Preparation

Compounds of the invention, in particular, the compounds listed in Table 2 supr., can be synthesized by methods described in this section, the examples, and the chemical literature.

### A. Preparation of Intermediates

### Preparation of 3(R)-Pyrrolidinyloxyamine dihydrochloride (Intermediate (I))

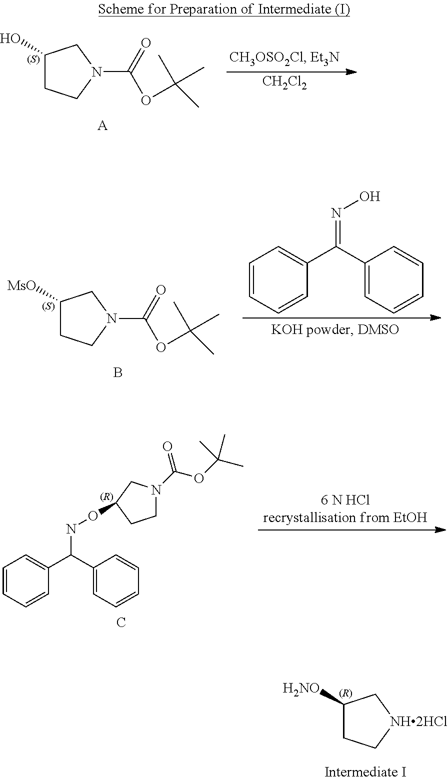

To a solution of N-tert-butoxycarbonyl-(S)-pyrrolidinol (A) (10.0 g) and triethylamine (8.2 mL) in CH₂Cl₂ (150 mL) at 0° C., methanesulfonyl chloride (4.34 mL) was added. After stirring at room temperature for 3 h, the reaction mixture was poured into ice/water and extracted with CH₂Cl₂. The organic phase was washed with 5% aqueous NaHCO₃, water, and brine, dried and evaporated to dryness to give an oil which solidified after standing overnight in the refrigerator. The solid was triturated with Et₂O to give N-tert-butoxycarbonyl-(S)-3-pyrrolidinyl methansulfonate (13.0 g, 92%) (Compound B) as a light yellow solid. ¹H-NMR (300 MHz, DMSO-d₆, ppm from TMS): δ 5.23 (1H, m), 3.60-3.10 (4H, m), 3.23 (3H, s), 2.11 (2H, m), 1.39 (9H, s).

To a suspension of KOH powder (4.86 g) in DMSO (250 mL) under vigorous stirring, benzophenone oxime (7.86 g) was added. After stirring at room temperature for 30 min, a solution of N-tert-butoxycarbonyl-(S)-3-pyrrolidinyl methansulfonate (10 g) (Compound B) in DMSO (70 mL) was added. After 18 h at room temperature, the reaction was poured into iced water (900 mL) and extracted with Et₂O. The combined organic layers were washed with water and brine, dried and the solvent evaporated. Benzophenone O—[(R)-3-pyrrolidinyl]oxime (Compound C) was obtained (13.0 g, 96%) as a white solid, which was used in the next step without further purification. ¹H-NMR (300 MHz, DMSO-d₆, ppm from TMS): δ 7.50-7.20 (10H, m), 4.84 (1H, m), 3.50-3.00 (4H, m), 2.01 (2H, m), 1.38 (9H, s).

Compound C (13.0 g) was suspended in 6N HCl (250 mL) and the mixture was refluxed for 2 h. After cooling, the reaction was extracted with Et₂O. The aqueous layer was evaporated to give a crude brown solid which was treated with 0.34 g of activated carbon in absolute EtOH (255 mL) at reflux for 2 h. The solid obtained after evaporation was crystallized with 96% EtOH (40 mL) to give the 3(R)-pyrrolidinyloxyamine dihydrochloride (Intermediate (I)) (2.98 g, 72%), as an off-white solid. ¹H-NMR (300 MHz, DMSO-d₆, ppm from TMS): δ 11.22 (3H, bb), 9.74 (1H, bb), 9.54 (1H, bb), 4.98 (1H, m), 3.60-3.00 (4H, m), 2.40-2.00 (2H, m).

### B. Preparation of Compounds of The Invention

### Example 1

### Synthesis of the Oxalate Salt of Compound 2: (5S,10S,13S,16R)-16a-fluoro-10,13-dimethyl-3-((S)-pyrrolidin-3-yloxyimino)-tetradecahydro-2H-cyclopenta[a]phenanthren-17(14H)-one

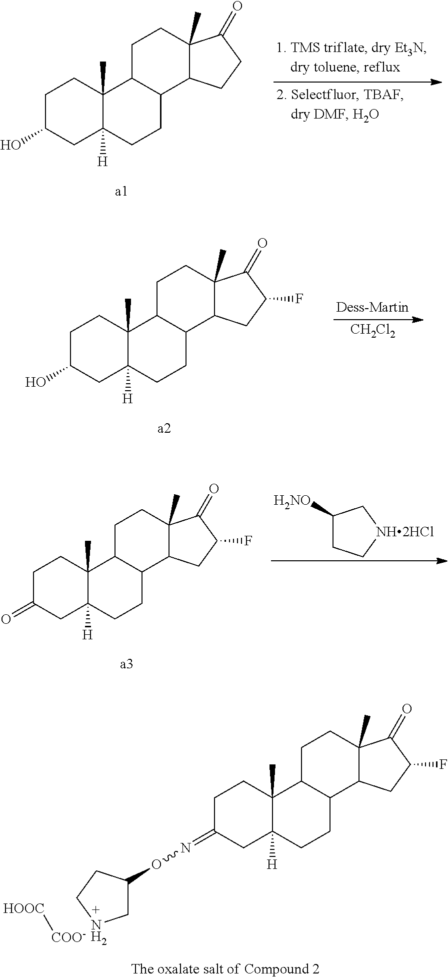

To a solution of 3α-hydroxy-5α-androstane-17-one (compound al) (200 mg) in dry toluene (1.2 mL) under Ar atmosphere, dry triethylamine (2.3 mL) and TMS triflate (0.4 mL) were added and the mixture was refluxed for 2 hours. After cooling to room temperature, hexane (20 mL) was added and the mixture was washed with sat. NaHCO₃. The organic layer was dried over anhydrous sodium sulphate and evaporated to dryness under reduced pressure. The yellow residue was dissolved in dry DMF (10 mL) and F-TEDA-BF₄ (270 mg) was added, under inert atmosphere. The mixture was stirred for 40 minutes and TBAF (0.75 mL, 1M in THF) was added. After 10 minutes, the reaction mixture was slowly poured with stirring into 25 mL H₂O, and a white solid precipitated out. After 2 hours, the mixture was filtered through a celite pad and washed with water to give 16α-fluoro-3α-hydroxy-5α-androstane-17-one (compound a2) (170 mg, 80%) as a white solid. ¹H-NMR (300 MHz, CDCl₃) δ 5.09 (dd, 1H, J=50.7 Hz, 6 Hz, CHF), 4.06 (m, 1H, CHOH), 2.20-0.81 (m, 21H), 0.91 (s, 3H), 0.81 (s, 3H).

Compound a2 (170 mg) was dissolved in dichloromethane (40 mL) and cooled to 0° C. Dess-Martin periodinane (585 mg) was added and the mixture was allowed to warm to room temperature. After 2 hours, the reaction mixture was quenched with a mixture of sat. NaHCO₃/Na₂S₂O₃ (1/3; 40 mL) and the resulting mixture was stirred for 45 minutes. The mixture was extracted with dichloromethane (3×15 mL) and washed with brine. The combined organic layers were dried over anhydrous sodium sulphate and evaporated to dryness. The residue was purified by flash chromatography (SiO₂, dichloromethane/diethyl ether 95/5) to give 16α-fluoro-5α-androstane-3,17-dione (compound a3) (130 mg, 78%) as a white solid. ¹H-NMR (300 MHz, CDCl₃) δ 5.10 (dd, 1H, J₁=50.4, 7.5 Hz, CHF), 2.45-0.81 (m, 20H), 1.04 (s, 3H), 0.94 (s, 3H). ¹⁹F-NMR (282.1 MHz, CDCl₃) δ −192.5 (m).

A solution of 3-(R)-pyrrolidinyloxyamine dihydrochloride (Intermediate I; 110 mg) in H₂O (3.1 mL) was slowly added to a solution of 16α-fluoro-5α-androstane-3,17-dione (compound a3) (130 mg) in THF (4.2 mL). After 2.5 hours, NaCl (250 mg) was added and the mixture was stirred for 15 minutes. The mixture was extracted with THF (3×10 mL) and the combined organic layers were dried over sodium sulphate and evaporated to dryness under reduced pressure. Then, chloroform was added and the mixture was washed with H₂O/NaOH 1N (pH=10). The combined organic layers were dried over anhydrous sodium sulphate and evaporated to dryness under reduced pressure. The residue was dissolved in ethyl acetate/methanol (8:2) and was treated with stoichiometric amount of oxalic acid (53 mg) to give the oxalate salt of Compound 2 (140 mg, 70%) as a pale white solid. ¹H-NMR (600 MHz, DMSO) δ 5.31 (dd, 1H, J₁=50.4, 7.2 Hz, CHF), 4.73 (m, 1H), 3.28 (m, 3H), 3.16-3.14 (m, 1H), 3.03-3.01 (m, 0.5H), 2.82-2.80 (m, 0.5H), 2.18 (m, 1H), 2.04-0.74 (m, 23H), 0.88 (s, 3H), 0.87 (s, 3H). ¹³C-NMR (75 MHz, CDCl₃) δ 212.8, 164.4, 160.9, 90.2 (d, J=182 Hz, CF), 79.4, 52.8, 49.3, 47.7, 47.1, 46.1, 43.3, 36.6, 35.8, 33.9, 33.6, 30.9, 29.8, 27.7, 27.0, 20.9, 19.5, 13.4, 11.0. ¹⁹F-NMR (282.1 MHz, CDCl₃) δ −181.0 (m), −192.1 (m).

### Example 2

### Synthesis of the Oxalate Salt of Compound 4: (10R,13S,16R)-16a-fluoro-10,13-dimethyl-3-((S)-pyrrolidin-3-yloxyimino)-tetradecahydro-14H-cyclopenta[a]phenanthrene-6,17-dione

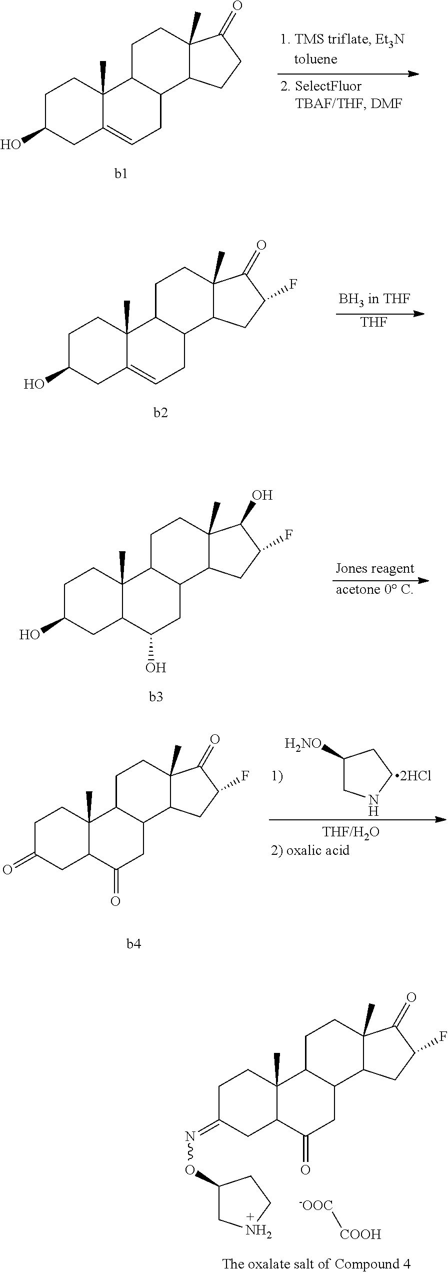

To a stirred solution of DHEA (compound b1) (3.0 g, 10.40 mmol) in dry toluene (18 mL), Et₃N (3.48 mL, 24.96 mmol) and TMS triflate (5.65 mL, 31.2 mmol) were added and the reaction mixture was refluxed at 100° C. for 5 h. The reaction mixture was cooled to room temperature, hexane (250 mL) was added and the mixture was extracted with sat. aq. NaHCO₃ (150 mL), dried over Na₂SO₄, filtered and evaporated to dryness under reduced pressure. The residue was dissolved in dry DMF (160 mL) and SelectFluor (4.42 g, 12.48 mmol) was added under Ar at room temperature. After 60 min., TBAF in THF (12.48 mL, 12.48 mmol) was added, and the reaction mixture was stirred for 15 min and then poured slowly into 250 mL of H₂O with stirring. The resulting precipitate was stirred for 2 h, filtered and washed with H₂O to obtain the product, which was further purified by column chromatography (ethyl acetate/hexane 4/6) which afforded 3.04 g (95%) of (3S,10R,13S,16R)-16-fluoro-3-hydroxy-10,13-dimethyl-1,3,4,7,8,9,10,11,12,13,15,16-dodecahydro-2H-cyclopenta[a]phenanthren-17(14H)-one (i.e., 16α-fluoro-3β-hydroxy-5-androsten-17-one; compound b2) as a white solid. ¹H-NMR (300 MHz, CDCl₃) δ 5.35 (1H, s), 5.08 (1H, dd, J=48.0, 6.0 Hz), 3.51 (1H, m), 2.49-1.07 (18H, m), 1.01 (3H, s), 0.92 (3H, s).

To a stirred solution of compound b2 (200 mg, 0.65 mmol) in anhydrous THF (5 mL) at −10° C. under Ar was added a solution of BH₃ in THF (1M) (1.96 mL, 1.96 mmol), and the reaction mixture was stirred at room temperature for 3.5 h. Water (6 mL) was added dropwise, followed by the addition of NaBO₃.4H₂O (201 mg, 1.31 mmol). The reaction mixture was stirred overnight at room temperature. The white precipitate was filtered through a celite pad, washed with THF and the filtrate was transferred into a separatory funnel. The two layers were separated and the aqueous layer was washed with THF (×3). The combined organic layers were dried over anhydrous Na₂SO₄, filtered and evaporated under reduced pressure. The residue was recrystallised from MeOH/AcOEt 1/5 to afford (3S,6S,10R,13S,16R,17R)-16α-fluoro-10,13-dimethyl-hexadecahydro-1H-cyclopenta[a]phenanthrene-3,6,17-triol (compound b3) as a white solid (120 mg, 57%). ¹H-NMR (300 MHz, CD₃OD) δ 4.84 (1H, ddd, J=54.2, 6.9, 4.7 Hz), 3.63 (1H, dd, J=29.2, 4.7 Hz), 3.47 (1H, m), 3.33 (1H, m), 2.19-0.65 (18H, m), 0.85 (3H, s), 0.73 (3H, s). ¹⁹F-NMR (282.1 MHz, CD₃OD) δ −180.8 (m).

To a stirred solution of compound b3 (100 mg, 0.31 mmol) in acetone (10 mL) at 0° C., Jones reagent was added dropwise until no further decolorization was observed (˜600 μL). At that point, i-PrOH was added at 0° C. until the Jones reagent was completely dissolved and the reaction mixture became blue (˜2 mL). The reaction mixture was stirred for an additional 15 min, filtered through a celite pad, washed with ethyl acetate and the filtrate was extracted with water. The aqueous layer was washed with ethyl acetate (×3) and the combined organic layers were dried over anhydrous Na₂SO₄, was filtered and evaporated under reduced pressure. The residue was purified by flash column chromatography (elution solvent cyclohexane/ethyl acetate 6/4) to afford (10R,13S,16R)-16α-fluoro-10,13-dimethyl-dodecahydro-2H-cyclopenta[a]phenanthrene-3,6,17(14H)-trione (compound b4) in the amount of 80 mg (yield: 81%) as pale yellow solid. ¹H-NMR (300 MHz, CDCl₃) δ 5.11 (1H, dd, J =51.0, 6.5 Hz), 2.68-0.85 (18H, m), 0.99 (3H, s), 0.95 (3H, s). ¹⁹F-NMR (282.1 MHz, CDCl₃) δ −192.2 (m)

A solution of 3-(R)-pyrrolidinyloxyamine dihydrochloride (Intermediate I; 24 mg, 0.137 mmol) in H₂O (0.75 mL) was slowly added to a solution of compound b4 (40 mg, 0.125 mmol) in THF (1.7 mL). After 1 h NaCl (47 mg, 0.811 mmol) was added, the reaction mixture was stirred for an additional 15 min and the two layers were separated. The aqueous layer was washed with THF (×2) and the combined organic phases were dried over anhydrous Na₂SO₄, filtered and evaporated under reduced pressure. The residue was purified by flash column chromatography (elution solvent chloroform/methanol/ammonia 90/10/1) to afford 36 mg (71%) of (10R,13S,16R)-16-fluoro-10,13-dimethyl-3-((S)-pyrrolidin-3-yloxyimino)-tetradecahydro-14H-cyclopenta[a]phenanthrene-6,17-dione (Compound 4) as a pale yellow solid. ¹H-NMR (600 MHz, CDCl₃) δ 5.36 (1H, bs), 5.08 (1H, dd, J=50.5, 7.1 Hz), 4.74 (1H, s), 3.32-3.04 (5H, m), 2.56-1.22 (19H, m), 0.91 (3H, s), 0.84 (3H, s). ¹³C NMR (75 MHz, CDCl₃) δ 212.0, 211.9, 209.1, 208.6, 159.4, 159.0, 91.1, 88.6, 58.0, 56.6, 53.5, 53.3, 48.6, 48.5, 48.1, 45.1, 41.8, 38.0, 37.1, 37.1, 37.0, 30.9, 29.9, 29.6, 27.3, 26.9, 20.8, 20.7, 20.4, 20.4, 14.6, 14.1, 12.6, 12.6. ¹⁹F-NMR (282.1 MHz, CDCl₃) δ −184.9 (m), −192.3 (m).

To obtain the oxalate salt, compound 4 as above obtained was dissolved in MeOH (0.1 mL), followed by the addition of an equimolar amount of oxalic acid. The corresponding oxalate salt was precipitated by the addition of diethyl ether. The precipitates were then filtered and dried to afford 25 mg of the oxalate salt of Compound 4.

### Example 3

### Synthesis of the Oxalate Salt of Compound 6: (5S,10R,13S,16R)-16a-fluoro-6-methylene-10,13-dimethyl-3-((R)-pyrrolidin-3-yloxyimino)-tetradecahydro-2H-cyclopenta[a]phenanthren-17(14H)-one

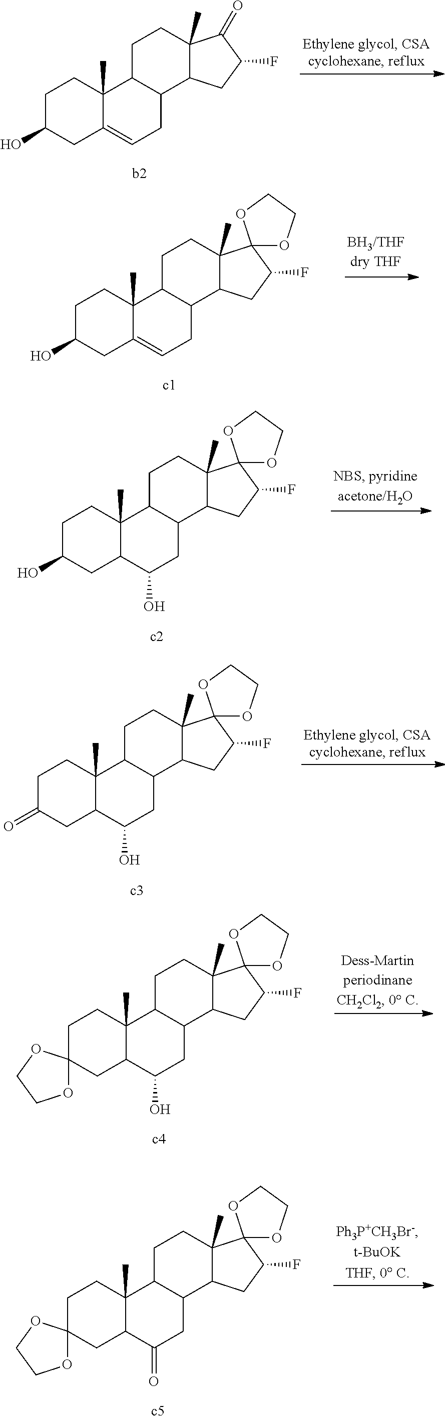 [SMILES: C[C@]12CC[C@H](O)CC1=CCC1C2CC[C@]2(C)C(=O)[C@H](F)CC12 ; C[C@]12CC[C@H](O)CC1=CCC1C2CC[C@@]2(C)C1C[C@@H](F)C21OCCO1 ; C[C@]12CC[C@H](O)CC1[C@@H](O)CC1C2CC[C@@]2(C)C1C[C@@H](F)C21OCCO1 ; C[C@]12CCC(=O)CC1[C@@H](O)CC1C2CC[C@@]2(C)C1C[C@@H](F)C21OCCO1 ; C[C@]12CCC3(CC1[C@@H](O)CC1C2CC[C@@]2(C)C1C[C@@H](F)C21OCCO1)OCCO3 ; C[C@]12CCC3(CC1C(=O)CC1C2CC[C@@]2(C)C1C[C@@H](F)C21OCCO1)OCCO3]

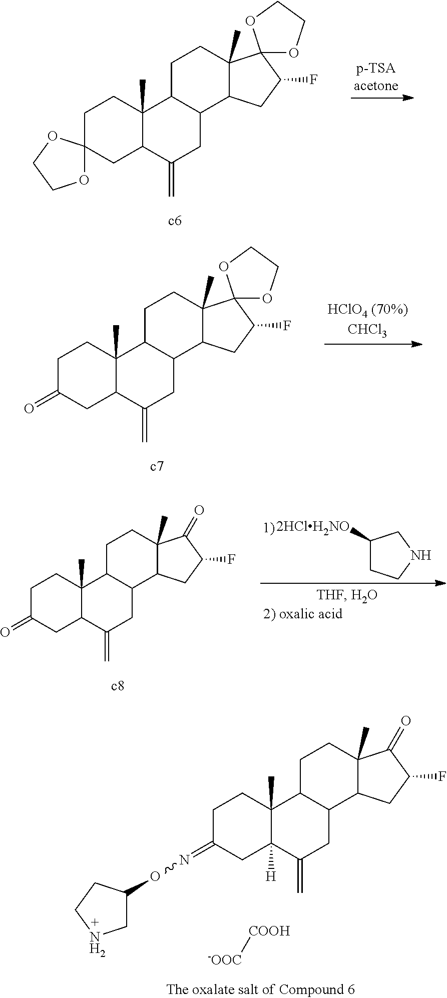

A mixture of 16α-fluoro-5-dehydroepiandrosterone (compound b2) (2.0 g) and ethylene glycol (16.4 mL) in cyclohexane (110 mL) was heated at 85° C. Then CSA (46 mg) was added and the mixture was stirred at reflux (105° C.) with a Dean-Stark trap, overnight. After cooling to room temperature, the mixture was diluted with ether (30 mL) and washed with 5% NaHCO₃ and brine. The organic layer was dried over anhydrous sodium sulfate and evaporated to dryness under reduced pressure. The crude product c1 (2.36 g) was pure enough to be used for the next step without any more purification. ¹H-NMR (600 MHz, CDCl₃) δ 5.32 (m, 1H), 5.02 (dm, J=52.8 Hz, 1H, CHF), 4.06-3.84 (m, 4H), 3.53-3.48 (m, 1H), 2.29-2.19 (m, 2H), 2.00-0.84 (m, 16H), 0.99 (s, 3H), 0.84 (s, 3H).

A solution of BH₃.THF (1M in THF, 13.1 mL) was added at −10° C. to a solution of crude cl (2.29 g) in anhydrous THF (50 mL) under argon atmosphere. The mixture was stirred at room temperature for 3 hours. Subsequently, 60 mL of H₂O were carefully added, followed by the addition of NaBO₃.4H₂O (2.01 g) and the mixture was stirred overnight. The mixture was filtered and the white solid was washed with cold THF. Then NaCl was added for the separation of the layers and the organic layer was extracted, dried over anhydrous sodium sulphate and evaporated to dryness. The residue was purified by flash chromatography (Petroleum ether/EtOAc 7/3) to afford 1.5 g (75%) of compound c2 as a white solid. ¹H-NMR (600 MHz, CDCl₃) δ 5.02 (dm, J=51.6 Hz, 1H, CHF), 4.09-3.84 (m, 4H), 3.61-3.56 (m, 1H), 3.42-3.38 (m, 1H), 2.20-2.17 (m, 1H), 1.97-1.93 (m, 1H), 1.82-0.72 (m, 18H), 0.83 (s, 3H), 0.81 (s, 3H). ¹⁹F-NMR (282.1 MHz, CDCl₃) δ −195.0 (m).

To a solution of compound c2 (1.42 g) in acetone/water 5/1 (120 mL), pyridine (5 mL) and NBS (1.37 g) were added and the mixture was stirred at room temperature overnight. The reaction mixture was quenched with conc. HCl to pH 3. After 10 minutes, NaOH 1N was added to make the mixture to pH=8. The organic solvent was evaporated to dryness and the aqueous layer was extracted with EtOAc (3×20 mL). The combined organic layers were dried over anhydrous sodium sulphate and evaporated to dryness. The residue was purified by flash chromatography (Chloroform/Methanol 95/5) to afford 1.1 g (78%) of compound c3 as a white solid. ¹H-NMR (600 MHz, CDCl₃) δ 4.98 (dm, J=55.8 Hz, 1H, CHF), 4.06-3.79 (m, 4H), 3.39-3.37 (m, 1H), 2.68-2.64 (m, 1H), 2.43-0.77 (m, 18H), 0.96 (s, 3H), 0.80 (s, 3H).

¹⁹F-NMR (282.1 MHz, CDCl₃) δ −195.0 (m). ¹³C-NMR (75 MHz, CDCl₃) δ 211.7, 115.3 (d, J=15 Hz), 97.3 (d, J=188 Hz), 77.2, 69.2, 65.8, 65.7, 65.3, 52.9, 52.8, 52.7, 47.6, 45.0, 40.3, 39.3, 38.2, 37.5, 36.3, 33.8, 30.7, 30.4, 29.2, 19.8, 14.4, 12.5.

Ketalisation of the ketone in compound c3 (1.1 g) was carried out following the above procedure for making compound cl. The corresponding diketal c4 was obtained in the amount of 1.32 g. The crude product c4 was used in the next step without further purification.

Diketal c4 (1.24 g) was dissolved in dichloromethane (65 mL) and the solution was cooled to 0° C. Dess-Martin periodinane (2.56 g) was added, and the mixture was warmed to room temperature. After 2 hours, the reaction mixture was quenched with a mixture of sat. NaHCO₃/Na₂S₂O₃ 1/3 (65 mL) and was stirred for 45 minutes. The mixture was extracted with dichloromethane (3×30 mL) and the organic layer was washed with brine. The combined organic layers were dried over anhydrous sodium sulphate and evaporated to dryness. The residue was purified by flash column chromatography (SiO₂, cyclohexane/ethyl acetate 75/25) to give compound c5 (1 g, 81% combined yield from compound c3) as a white solid. ¹H-NMR (600 MHz, CDCl₃) δ 4.97 (dm, J=53.4 Hz, 1H, CHF), 4.03-3.80 (m, 8H), 2.47 (dd, J₁=11.1, 4.2 Hz, 1H), 2.38-2.25 (m, 1H), 2.22 (dd, J₁=13.5, 4.2 Hz, 1H), 1.98-1.89 (m, 2H), 1.81-0.70 (m, 13H), 0.78 (s, 3H), 0.70 (s, 3H). ¹⁹F-NMR (282.1 MHz, CDCl₃) δ −195.0 (m). ¹³C-NMR (75 MHz, CDCl₃) δ 210.3, 115.2 (d, J=15 Hz), 108.7, 97.1, (d, J=188 Hz), 65.9, 65.8, 65.3, 64.1, 64.0, 55.8, 52.9, 48.3, 45.5, 45.4, 40.6, 37.2, 35.4, 30.5, 30.4, 30.2, 29.6, 29.1, 20.0, 14.4, 12.3.

To a stirred solution of the methyl triphenyl phosphonium bromide (1.11 mg, 3.12 mmol) in dry THF (8.5 mL) at 0° C. under Ar, t-BuOK (338 mg, 3.01 mmol) was added and the resulting solution was stirred for 10 min, at which point a solution of compound c5 (300 mg, 0.734 mmol) in THF (8.5 mL) was added dropwise during 5 min. The reaction mixture was stirred for 30 min at room temperature under Ar, followed by the addition of 5% NaH₂PO₄ (10 mL). The organic solvent was then evaporated. The aqueous layer was extracted with Et₂O (×3) and the combined organic layers were washed with brine, dried over Na₂SO₄, filtered and evaporated under reduced pressure. The residue was purified by flash column chromatography (elution solvent cyclohexane/ethyl acetate 9/1) to afford 290 mg (97%) of compound c6 as a white solid. ¹H-NMR (600 MHz, CDCl₃) δ 5.07 (0.5H, m), 4.89 (0.5H, m), 4.66 (1H, s), 4.37 (1H, s), 4.05-3.81 (8H, m), 2.21-0.93 (18H, m), 0.78 (3H, s), 0.64 (3H, s). ¹³C NMR (75 MHz, CDCl₃) δ 148.9, 115.50 (d, J=14.9 Hz), 109.4, 105.8, 97.50 (d, J=192.3 Hz), 77.2, 65.9, 65.9, 65.3, 64.1, 64.1, 53.8, 48.1, 48.0, 45.3, 45.2, 40.9, 37.6, 37.6, 37.0, 35.3, 33.4, 30.8, 30.7, 30.4, 29.4, 20.0, 14.5, 11.5. ¹⁹F-NMR (282.1 MHz, CDCl₃) δ −195.0 (m).

A mixture of compound c6 (260 mg, 0.64 mmol) and p-TSA (490 mg, 2.55 mmol) in acetone (26 mL) was stirred at room temperature overnight. The solution was neutralized by addition of 5% NaHCO₃ and the organic solvent was evaporated to dryness. The residue was diluted with water and extracted with dichloromethane (3×10 mL). The combined organic layers were dried over anhydrous sodium sulfate and evaporated to dryness under reduced pressure to afford 195 mg (84%) of compound c7 as a white solid. ¹H-NMR (600 MHz, CDCl₃) δ 5.08 (0.5H, m), 5.00 (0.5H, m), 4.80 (1H, s), 4.44 (1H, s), 4.08-3.92 (4H, m), 2.48-0.99 (18H, m), 0.89 (3H, s), 0.85 (3H, s). ¹⁹F-NMR (282.1 MHz, CDCl₃) δ −195.0 (m).

Perchloric acid (70%, 0.4 mL) was added at −30° C. to a solution of compound c7 (80 mg, 0.221 mmol) in chloroform (10 mL). The mixture was stirred at the same temperature for 0.5 h and saturated NaHCO₃ was added. The crude product was extracted with chloroform (3×10 mL) and the combined organic layers were dried over anhydrous sodium sulfate and evaporated to dryness under reduced pressure. The residue was purified by flash column chromatography (SiO₂, petroleum ether/ethyl acetate 9/1) to afford 37 mg (53%) of compound c8 as a white solid. ¹H-NMR (600 MHz, CDCl₃) δ 5.09 (1H, dd, J=50.6, 7.5 Hz), 4.85 (1H, s), 4.48 (1H, s), 2.53-0.80 (18H, m), 0.92 (3H, s), 0.91 (3H, s). ¹³C NMR (75 MHz, CDCl₃) δ 212.7 (d, J=12.6 Hz), 211.7, 146.8, 107.8, 90.1 (d, J=187.0 Hz), 65.6, 54.1, 50.8, 48.2, 47.9, 40.6, 40.3, 38.2, 37.9, 37.8, 36.6, 31.2, 30.0, 29.7, 20.6, 14.2, 11.8. ¹⁹F-NMR (282.1 MHz, CDCl₃) δ −192.4 (m).

To a stirred solution of compound c8 (37 mg, 0.116 mmol) in THF (3.0 mL) was added dropwise a solution of 3-(R)-pyrrolidinyloxyamine dihydrochloride (Intermediate I; 22 mg, 0.128 mmol) in water (1.2 mL). After 45 min, NaCl (44 mg, 0.754 mmol) was added. The reaction mixture was stirred for an additional 15 min and extracted. The aqueous phase was washed with THF (×2) and the combined organic phases were dried over Na₂SO₄, filtered and evaporated under reduced pressure. The residue was purified by flash column chromatography (elution solvent chloroform/methanol/ammonia 90/10/1) to afford compound 6 in the amount of 23 mg (yield: 50%) as white solid. ¹H-NMR (300 MHz, CDCl₃) δ 5.09 (1H, dd, J=50.6, 7.0 Hz), 4.84 and 4.82 (1H, s), 4.73 (1H, m), 4.58 and 4.54 (1H, s), 3.27-2.84 (6H, m), 2.38-1.04 (18H, m), 0.91 (3H, s), 0.80 (3H, s). ¹⁹F-NMR (282.1 MHz, CDCl₃): δ −185.0, 192.5 ppm.

Compound 6 was then dissolved in MeOH (0.1 mL), and an equimolar amount of oxalic acid was added. The corresponding oxalate salt was precipitated by the addition of diethyl ether, filtered, and dried to give the oxalate salt of Compound 6.

### Example 4

### Synthesis of the Oxalate Salt of Compound 8: (5S,10R,13S,16R)-16a-fluoro-6-(methoxyimino)-10,13-dimethyl-3-((R)-pyrrolidin-3-yloxyimino)-tetradecahydro-2H-cyclopenta[a]phenanthren-17(14H)-one

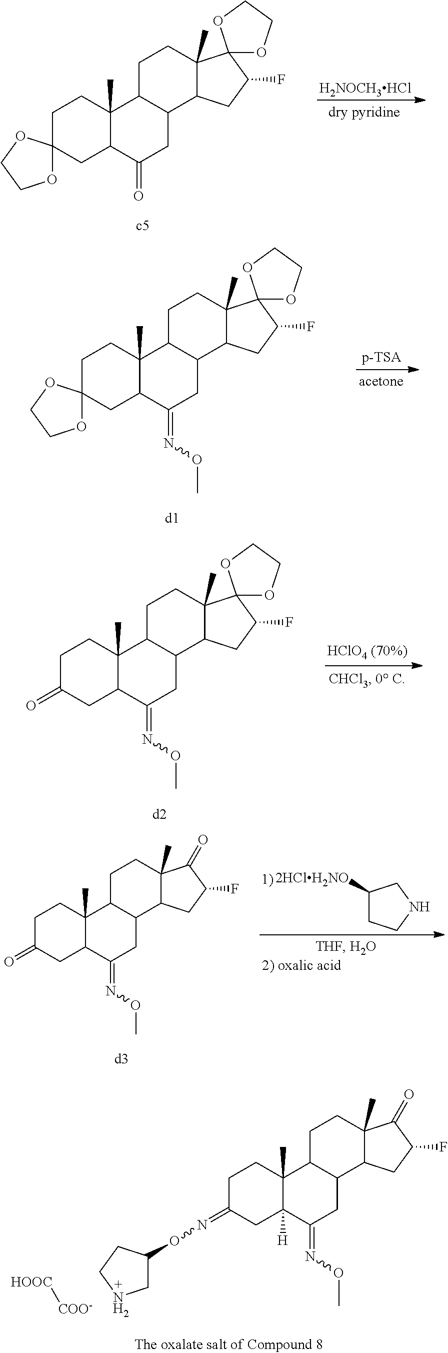

To a solution of compound c5 (200 mg) in dry pyridine (5 mL) under argon, methoxyamine hydrochloride (82 mg) was added and the mixture was stirred overnight at room temperature. Pyridine was evaporated and the residue was diluted with dichloromethane (20 mL) and H₂O (20 mL) and the layers were separated. The aqueous layer was extracted with dichloromethane (3×10 mL), and the combined organic layers were dried over anhydrous sodium sulfate and evaporated to dryness under reduced pressure. The residue was purified by flash column chromatography (SiO₂, petroleum ether/diethyl ether 7/3) to afford 190 mg (89%) of oxime dl as a white solid: ¹H-NMR (300 MHz, CDCl₃) δ 5.03 (dm, J=49.5 Hz, 1H), 4.06-3.86 (m, 8H), 3.79 (s, 3H), 3.25-3.15 (m, 1H), 2.24-2.19 (m, 1H), 2.03-0.74 (m, 16H), 0.82 (s, 3H), 0.74 (s, 3H). ¹⁹F-NMR (282.1 MHz, CDCl₃) δ −194.9 (m, CHF). ¹³C-NMR (75 MHz, CDCl₃) δ 158.3, 119.0, 115.4 (d, J=15 Hz, CCF), 109.2, 109.1, 97.4 (d, J=195 Hz, CF), 65.9, 65.7, 65.4, 65.1, 64.4, 64.1, 64.0, 61.1, 53.7, 53.5, 50.4, 48.3, 45.4, 38.5, 35.9, 35.3, 35.2, 35.0, 34.0, 31.2, 30.8, 30.7, 30.4, 30.3, 29.4, 29.3, 29.2, 22.4, 20.7, 20.0, 15.2, 14.5, 14.3, 11.7.

A mixture of oxime dl (190 mg) and p-TSA (330 mg) in acetone (18 mL) was stirred at room temperature overnight. Then the solution was neutralized with 5% NaHCO₃ and the organic solvent was evaporated to dryness. The mixture was diluted with water and extracted with dichloromethane (3×10 mL). The combined organic layers were dried over anhydrous sodium sulfate and evaporated to dryness under reduced pressure. The residue was purified by flash column chromatography (SiO₂, petroleum ether/ethyl acetate 9/1) to afford 144 mg (96%) of compound d2 as a white solid. ¹H-NMR (300 MHz, CDCl₃) δ 5.03 (dm, J=49.5 Hz, 1H), 4.07-3.91 (m, 4H), 3.78 (s, 3H), 3.21 (dd, J₁=13.8, 4.2 Hz, 1H), 2.63-0.84 (m, 17H), 0.92 (s, 3H), 0.84 (s, 3H). ¹⁹F-NMR (282.1 MHz, CDCl₃) δ −194.9 (m). ¹³C-NMR (75 MHz, CDCl₃) δ 210.8, 208.2, 171.1, 115.2 (d, J=15 Hz), 97.1 (d, J=195 Hz), 66.1, 66.0, 65.5, 60.3, 57.4, 53.0, 48.4, 45.6, 45.6, 45.5, 41.1, 37.8, 37.5, 37.2, 36.8, 30.6, 30.3, 29.6, 29.1, 21.0, 20.3, 14.5, 14.1, 12.5.

Perchloric acid (70%, 5 drops) was added at 0° C. to a solution of compound d2 (40 mg) in chloroform (10 mL). The mixture was stirred at the same temperature for 10 minutes. Then saturated NaHCO₃ was added and the crude product was extracted with chloroform (3×10 mL). The combined organic layers were dried over anhydrous sodium sulfate and evaporated to dryness under reduced pressure. The residue was purified by flash column chromatography (SiO₂, petroleum ether/ethyl acetate 7/3) to afford 27 mg (77%) of compound d3 as a white solid. ¹H-NMR (600 MHz, CDCl₃) δ 5.01 (dd, J=50.7, 7.8 Hz, 1H), 3.82 (s, 3H), 3.34 (dd, J=13.5, 4.2 Hz, 1H), 2.63-2.59 (m, 1H), 2.43-0.93 (m, 16H), 0.97 (s, 3H), 0.93 (s, 3H).

¹⁹F-NMR (282.1 MHz, CDCl₃) δ −192.3 (m).

To a solution of compound d3 (27 mg, 0.077 mmol) in THF (2.0 mL) was added a solution of 3-(R)-pyrrolidinyloxyamine dihydrochloride (Intermediate I; 13.5 mg, 0.077 mmol) in water (1 mL). After stirring at room temperature for 2 h, NaCl (230 mg) was added, and the reaction mixture was stirred for an additional 15 min. The two layers were separated and the aqueous layer was extracted with THF (×2). The combined organic phases were dried over anhydrous Na₂SO₄, filtered and evaporated in vacuo. The residue was dissolved in CHCl₃ and the organic layer was extracted with NaOH solution (pH 10), separated, dried over anhydrous Na₂SO₄ and evaporated in vacuo to afford compound 8 (29 mg, 87% yield).

To a solution of compound 8 (29 mg) in MeOH/AcOEt (2/8, 2 mL) an equimolar amount of oxalic acid was added. The corresponding oxalate salt was precipitated by the addition of diethyl ether, filtered and dried (22 mg). ¹H-NMR (600 MHz, CD₃OD) δ 5.13 (dd, J=50.7, 6.5 Hz, 1H), 4.83 (m, 1H), 3.79 (s, 3H), 3.56-3.52 (1H, m), 3.42-3.31 (5H, m), 3.21-3.12 (m, 1H), 2.37-1.1 (m, 18H), 0.95 (s, 3H), 0.88 and 0.87 (s, 3H); ¹⁹F-NMR (282.1 MHz, CDCl₃) δ −194.3 (m) and −187.5 (m); ESI-MS [M+1]⁺=434.3

### Example 5

### Synthesis of the Oxalate Salt of Compound 21: (10R,13S)-10,13-dimethyl-4-phenyl-3-((S)-pyrrolidin-3-yloxyimino)-tetradecahydro-2H-cyclopenta[a]phenanthren-17(14H)-one

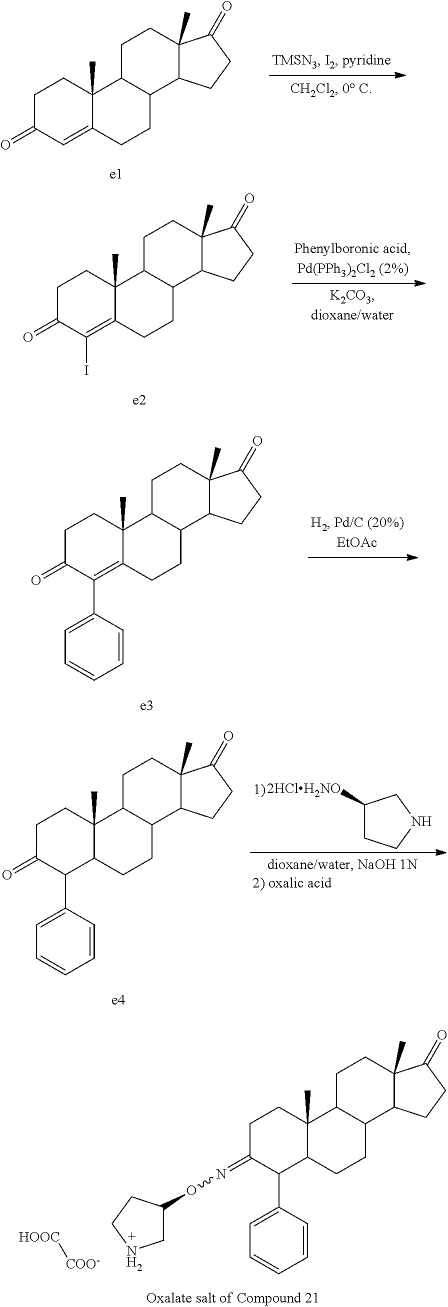

4-Androstene-3,17-dione (compound e1) (1 mmol, 300 mg) was dissolved in dichloromethane (5 mL) and the solution was cooled to 0° C. TMSN₃ (2.5 mmol, 0.33 mL) was added and the mixture was stirred for 2 hours at the same temperature. Then a solution of I₂ (2.5 mmol, 630 mg) in pyridine/dichloromethane 1:1 (10 mL) was added and the mixture was allowed to worm up to room temperature and was stirred overnight. Upon completion of the reaction, the mixture was diluted with ether (30 mL). The organic layer was separated, washed with H₂O (20 mL), 1N HCl (20 mL), saturated NaHCO₃ (20 mL), and aqueous Na₂S₂O₃ solution (20 mL). The organic layers were combined, dried over anhydrous sodium sulfate and evaporated to dryness under reduced pressure. The residue was purified by flash column chromatography (SiO₂, cyclohexane/ethyl acetate 8/2) to afford 400 mg (93%) of compound e2 as a white solid.

¹H-NMR (600 MHz, CDCl₃) δ 3.26 (dt, J₁=14.4, 3.0 Hz, 1H), 2.70 (dt, J₁=16.8, 4.2 Hz, 1H), 2.61-2.55 (m, 1H), 2.52-2.46 (m, 2H), 2.15-0.93 (m, 14H), 1.26 (s, 3H), 0.93 (s, 3H). ¹³C-NMR (75 MHz, CDCl₃) δ 220.0, 191.8, 173.1, 107.9, 54.0, 50.7, 47.5, 43.1, 39.6, 35.7, 35.0, 34.9, 33.0, 31.2, 30.4, 21.7, 20.6, 18.1, 13.7.

To a solution of dioxane/water 3/1 (10 mL) were sequentially added phenylboronic acid (90 mg), K₂CO₃ (165 mg), Pd(PPh₃)₂Cl₂ (2%, 9 mg) and compound e2 (250 mg) under argon. The mixture was stirred at 100° C. for 1.5 hour, then diluted with chloroform (20 mL) and water (10 mL). The aqueous layer was extracted with chloroform (3×10 mL). The combined organic layers were dried over anhydrous sodium sulfate and evaporated to dryness under reduced pressure. The residue was purified by flash column chromatography (SiO₂, petroleum ether/ethyl acetate 8/2) to afford 170 mg (78%) of compound e3 as a white solid. ¹H-NMR (600 MHz, CDCl₃) δ 7.35 (t, J=7.2 Hz, 2H), 7.30-7.27 (m, 1H), 7.01 (d, J=7.2 Hz, 2H), 2.62-0.93 (m, 19H), 1.32 (s, 3H), 0.93 (s, 3H).

A mixture of compound e3 (60 mg) and 10% Pd/C (12 mg) in EtOAc (5 mL) was stirred under H₂ atmosphere for 48 hours at room temperature. The mixture was filtered through a celite pad and the filtrate was evaporated to dryness. The crude product was purified with flash column chromatography (SiO₂, cyclohexane/ethyl acetate 8/2) to afford 40 mg of compound e4 (65%), as a white solid. ¹H-NMR (300 MHz, CDCl₃) δ 7.34-7.22 (m, 3H), 7.02 (d, J=6.0 Hz, 2H), 3.39 (d, J=12 Hz, 1H), 2.64-0.81 (m, 20H), 1.20 (s, 3H), 0.88 (s, 3H).

A solution of dioxane/H₂O (2/1, 1.5 mL) was treated with NaOH 1N to pH=12 and compound e4 (20 mg) was added, followed by a solution of 3-(R)-pyrrolidinyloxyamine dihydrochloride (Intermediate I; 9.6 mg) in H₂O (0.5 mL). After stirring for 3 hours at room temperature the organic solvent was evaporated and the mixture was diluted with water and extracted with chloroform (3×10 mL). The combined organic layers were dried over anhydrous sodium sulfate and evaporated to dryness under reduced pressure. The residue was purified by flash chromatography (SiO₂, chloroform/methanol/ammonia 9.25/0.5/0.25) to afford 10 mg of compound 21 as colourless oil, which was dissolved in ethyl acetate/methanol 8/2 and treated with oxalic acid (3 mg) to give 6.3 mg of the oxalate salt of compound 21 as a white solid.

¹H-NMR (600 MHz, CD₃OD) δ 7.29 (t, J=6 Hz, 2H), 7.22-7.14 (m, 3H), 4.66 (m, 1H), 3.36-3.29 (m, 2H), 3.22-3.17 (m, 3H), 3.01-2.96 (m, 1H), 2.11-0.83 (m, 24H), 1.09 (s, 3H), 0.87 (s, 3H).

### Example 6

### Synthesis of the Oxalate Salt of Compound 25: (5S,10R,13S)-10-(hydroxymethyl)-13-methyl-3-((S)-pyrrolidin-3-yloxyimino)-tetradecahydro-2H-cyclopenta[a]phenanthren-17(14H)-one

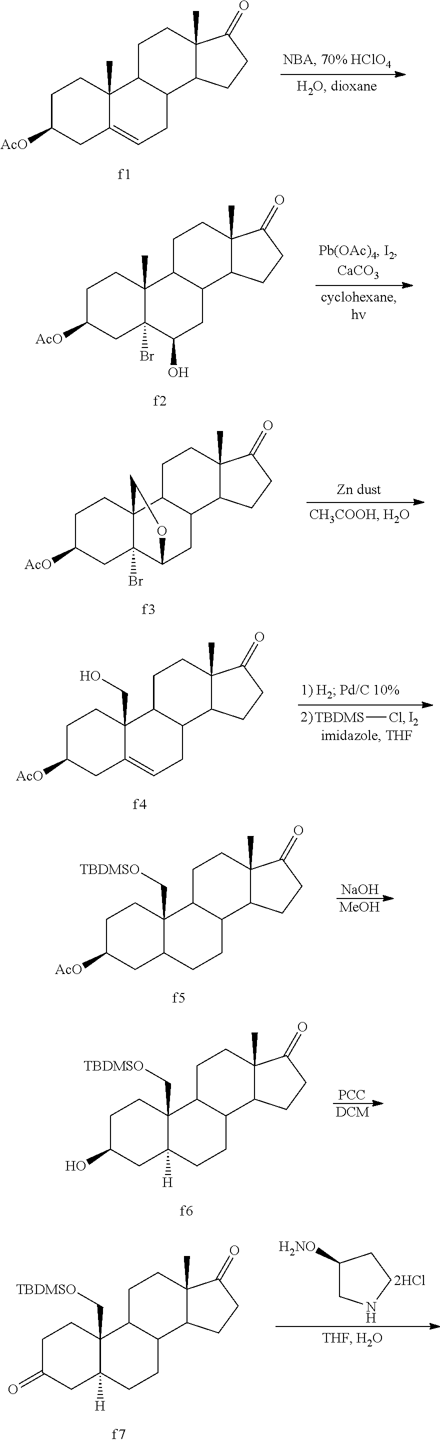

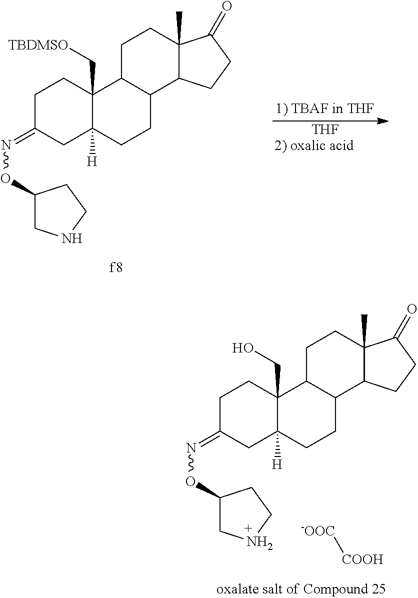

To a stirred solution of Ac-DHEA (compound f1) (10.89 g, 32.95 mmol) in dioxane (82 mL) and water (16.5 mL), a suspension of NBA (5.0 g, 36.24 mmol) in water (13 mL) was added. The reaction mixture became yellow and was stirred for 2 h at room temperature. Saturated aq. Na₂S₂O₃ was added and the reaction mixture was stirred for an additional 30 min, and subsequently was poured into ice-water. Chloroform was added and the two phases were separated. The organic phase was extracted with sat. aq. NaHCO₃ and brine, dried over Na₂SO₄, filtered and evaporated under reduced pressure. The residue was recrystallized by acetone/pentane 1/4 to afford 7.73 g (55%) of (3S,5R,6R,10R,13S)-5-bromo-6-hydroxy-10,13-dimethyl-17-oxo-hexadecahydro-1H-cyclopenta[a]phenanthren-3-yl acetate (compound f2).

¹H-NMR (600 MHz, CDCl₃) δ 5.47 (1H, m), 4.23 (1H, m), 2.52-1.31 (19H, m), 2.04 (3H, s), 1.35 (3H, s), 0.88 (3H, s); ¹³C-NMR (75 MHz, CDCl₃) δ 220.6, 170.4, 101.8, 86.0, 75.46, 75.42, 71.9, 71.8, 50.9, 50.7, 47.8, 47.6, 40.5, 38.3, 35.8, 35.0, 33.4, 31.3, 30.3, 26.2, 26.2, 21.6, 21.3, 20.5, 17.8, 13.9.

A suspension of compound f2 (5.00 g, 11.70 mmol), Pb(OAc)₄ (19.45 g, 43.88 mmol), powdered CaCO₃ (9.95 g, 99.45 mmol) and I₂ (6.53 g, 25.74 mmol) in cyclohexane (550 mL) was refluxed by irradiation with a 375 W lamp for 2.5 h. The reaction mixture was cooled to room temperature, filtered through celite and washed with cyclohexane. The filtrate was extracted with 5% aq. Na₂S₂O₃ and brine and the organic phase was dried over Na₂SO₄, filtered and evaporated under reduced pressure below 45° C. to afford 5.0 g of compound f3 as a yellow viscous liquid, which was used for the next step without any further purification.

To a stirred solution of crude compound f3 (4.98 g, 11.70 mmol) in acetic acid (150 mL) and water (10 mL), Zn dust (27.54 g, 421.2 mmol) was added in 3 portions. The reaction mixture was stirred at 45° C. for 1.5 h, filtered through celite, washed with water and DCM, and the filtrate was evaporated to dryness. The residue was dissolved in Et₂O, extracted with aq. NaHCO₃ and brine, dried over Na₂SO₄, filtered and evaporated. The residue was recrystallized from Et₂O to afford 1.21 g of (3S,10S,13S)-10-(hydroxymethyl)-13-methyl-17-oxo-2,3,4,7,8,9,10,11,12,13,14,15,16,17-tetradecahydro-1H-cyclopenta[a]phenanthren-3-yl acetate (compound f4) as a white solid, while an additional 1.93 g were isolated by flash column chromatography (elution solvent DCM/MeOH 99/1-97/3) (with a combined yield of 77% starting from compound f2).

¹H-NMR (300 MHz, CDCl₃) δ 5.74 (1H, s), 4.59 (1H, m), 3.82 and 3.58 (2H, AB system, J=7.0 Hz), 2.45-0.98 (19H, m), 1.98 (3H, s), 0.88 (3H, s).

To a solution of compound f4 (500 mg, 1.44 mmol) in MeOH (50 mL), 10% Pd/C (100 mg) was added, a balloon with hydrogen was applied and the mixture was stirred for 2 d at room temperature. The reaction mixture was filtered from a celite pad, and washed with MeOH. The filtrate was evaporated in vacuo, and the residue was purified by flash column chromatography (elution solvent CHCl₃/MeOH 98/2) to afford (3S,10R,13S)-10-(hydroxymethyl)-13-methyl-17-oxo-hexadecahydro-1H-cyclopenta[a]phenanthren-3-yl acetate as a white solid, 492 mg (98% yield). ¹H-NMR (600 MHz, CDCl₃) δ 4.73 (1H, m), 3.93 and 3.83 (2H, AB system, J=11.5 Hz), 2.46-0.74 (22H, m), 2.02 (3H, s), 0.89 (3H, s). ¹³C-NMR (75 MHz, CDCl₃) δ 221.1, 170.6, 73.1, 60.6, 54.6, 51.6, 47.8, 44.7, 39.2, 35.7, 35.5, 34.3, 31.9, 31.0, 30.7, 27.8, 22.0, 21.7, 21.3, 13.9.

To a stirred solution of (3S,10R,13S)-10-(hydroxymethyl)-13-methyl-17-oxo-hexadecahydro-1H-cyclopenta[a]phenanthren-3-yl acetate (480 mg, 1.38 mmol) in dry THF (5 mL) at 0° C. were added imidazole (282 mg, 4.14 mmol) and I₂ (1.05 g, 4.14 mmol). The reaction mixture was stirred for 30 min. Subsequently, TBDMS-Cl (229 mg, 1.52 mmol) was added and the resulting mixture was stirred for 2 d at room temperature. The reaction mixture was diluted by addition of ethyl acetate and the organic layer was extracted with aq. Na₂S₂O₃ (×2) and brine, was dried over anhydrous Na₂SO₄, was filtered and evaporated in vacuo. The residue was purified by flash column chromatography (elution solvent, chloroform) to afford (3S,10S,13S)-10-((tert-butyldimethylsilyloxy)methyl)-13-methyl-17-oxo-2,3,4,7,8,9,10,11,12,13,14,15,16,17-tetradecahydro-1H-cyclopenta[a]phenanthren-3-yl acetate (compound f5) as a white solid (440 mg, 69% yield). ¹H-NMR (600 MHz, CDCl₃) δ 4.71 (1H, m), 3.85 and 3.69 (2H, AB system, J=10.6 Hz), 2.45-0.70 (22H, m), 2.02 (3H, s), 0.89 (9H, s), 0.86 (3H, s), 0.07 (6H, s).

To a solution of compound f5 (420 mg, 0.91 mmol) in MeOH (14 mL), was added a solution of NaOH 1M (5.0 mL, 5.0 mmol). The resulting mixture was stirred overnight at room temperature. Ice-water (50 mL) was added, and the reaction mixture was filtered. The solid was washed with water and was dried to afford (3S,5S,10R,13S)-10-((tert-butyldimethylsilyloxy)methyl)-3-hydroxy-13-methyl-tetradecahydro-2H-cyclopenta[a]phenanthren-17(14H)-one (compound f6) (378 mg). ¹H-NMR (600 MHz, CDCl₃) δ 3.85 and 3.68 (2H, AB system, J=10.6 Hz), 3.64 (1H, m), 2.46-0.69 (22H, m), 0.89 (9H, s), 0.87 (3H, s), 0.07 (6H, s).

To a stirred solution of compound f6 (378 mg, 0.91 mmol) in DCM (3 mL) a suspension of PCC (424 mg, 1.96 mmol) in DCM (5 mL) was added and the reaction mixture was stirred for 2 h at room temperature. The reaction mixture was filtered through a celite pad, washed with DCM. The filtrate was washed with brine, was dried over anhydrous Na₂SO₄, was filtered and evaporated. The residue was purified by flash column chromatography (elution solvent, petroleum ether/ethyl acetate 8/2) to afford (5S,10R,13S)-10-((tert-butyldimethylsilyloxy)methyl)-13-methyl-dodecahydro-2H-cyclopenta[a]phenanthrene-3,17(4H,14H)-dione (compound f7) as a white solid, 260 mg (a combined yield of 68% for the previous two steps). ¹H-NMR (300 MHz, CDCl₃) δ 3.95 and 3.90 (2H, AB system, J=10.8 Hz), 2.50-0.77 (22H, m), 0.89 (12H, s), 0.09 (3H, s), 0.08 (3H, s).

A solution of 3-(R)-pyrrolidinyloxyamine dihydrochloride (Intermediate I; 33 mg, 0.19 mmol) in water (0.6 mL) was added to a solution of compound f7 (80 mg, 0.19 mmol) in THF (1.3 mL). After 2 h NaCl (72 mg, 1.24 mmol) was added, the reaction mixture was stirred for an additional 15 min and the two layers were separated. The aqueous layer was extracted with THF (×2) and the combined organic layers were dried over Na₂SO₄, filtered and evaporated in vacuo to afford (5S,10R,13S)-10-((tert-butyldimethylsilyloxy)methyl)-13-methyl-3-((S)-pyrrolidin-3-yloxyimino)-tetradecahydro-2H-cyclopenta[a]phenanthren-17(14H)-one (compound f8), which was carried into the next step without purification.

To a solution of the crude f8 (98 mg, 0.19 mmol) in dry THF (1.8 mL) under Ar, a 1M solution of TBAF in THF (0.57 mL, 0.57 mmol) was added and the reaction mixture was stirred at room temperature for 1 h. After the reaction was completed, ice-water was added and the mixture was extracted with chloroform, dried over Na₂SO₄, filtered and the solvent was evaporated in vacuo. The residue was purified by flash column chromatography (elution solvent CHCl₃/MeOH/NH₃ 95/5/1→90/10/1) to afford (5S,10R,13S)-10-(hydroxymethyl)-13-methyl-3-((S)-pyrrolidin-3-yloxyimino)-tetradecahydro-2H-cyclopenta[a]phenanthren-17(14H)-one (Compound 25) 49 mg (65% yield from compound f7). ¹H-NMR (600 MHz, CD₃OD) δ 4.83 (1H, s), 3.89 (2H, AB, J=11.7 Hz), 3.54-2.92 (6H, m), 2.44-0.77 (18H, m), 0.91 (3H, s). ¹³C-NMR (75 MHz, CD₃OD) δ 221.2, 221.1, 162.8, 162.2, 79.4, 79.3, 77.2, 60.4, 59.6, 54.3, 51.6, 50.0, 49.8, 47.8, 45.8, 44.1, 44.0, 43.9, 39.8, 39.7, 35.7, 35.4, 35.2, 34.1, 32.8, 31.9, 31.8, 30.5, 30.1, 28.6, 28.0, 27.9, 22.2, 21.7, 21.6.

Compound 25 was dissolved in MeOH (0.1 mL), and an equimolar amount of oxalic acid was added. The corresponding salt was precipitated by the addition of diethyl ether, filtered and dried to yield the oxalate salt of Compound 25: ¹H-NMR (600 MHz, CD₃OD) δ 4.83 (1H, s), 3.91 (2H, AB, J=11.6 Hz), 3.55-2.94 (6H, m), 2.46-0.79 (18H, m), 0.92 (3H, s).

### Example 7

### Synthesis of the Oxalate Salt of Compound 28: (5S,10S,13S)-10-(hydroxymethyl)-13-methyl-3-((S)-pyrrolidin-3-yloxyimino)-tetradecahydro-14H-cyclopenta[a]phenanthrene-6,17-dione

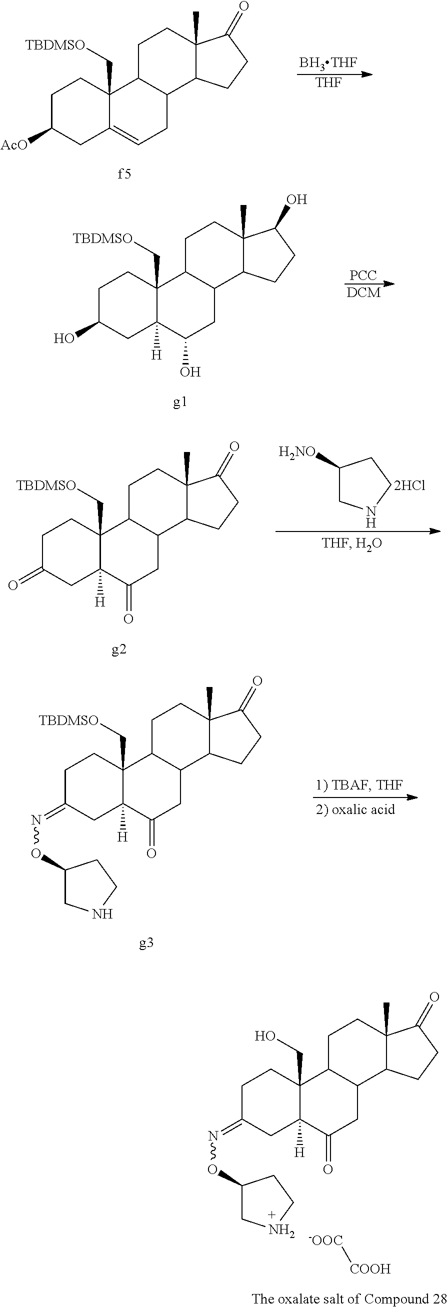

To a stirred solution of compound f5 (1.21 g, 2.63 mmol) in dry THF (15 mL) at −10° C., BH₃ (1M) in THF (7.88 mL, 7.88 mmol) was added and the reaction mixture was stirred for 3 h at room temperature. Water (15 mL) was added dropwise, followed by the addition of NaBO₃.4H₂O (0.808 g, 5.25 mmol). The reaction mixture was stirred overnight at room temperature. The mixture was then filtrated and washed with THF, and the two phases of the filtrate were separated. The aqueous phase was extracted with THF (×3) and the combined organic phases were dried over anhydrous Na₂SO₄, filtered and evaporated. The residue was purified by flash column chromatography (elution solvent, chloroform/MeOH 98/24→95/5) to afford 700 mg (61%) of (3S,5S,6S,10S,13S,17S)-10-((tert-butyldimethylsilyloxy)methyl)-13-methyl-hexadecahydro-1H-cyclopenta[a]phenanthrene-3,6,17-triol (compound g1) as a white solid. ¹H-NMR (300 MHz, CD₃OD) δ 3.81 and 3.68 (2H, AB system, J=10.7 Hz), 3.53 (1H, t), 3.47 (1H, m), 3.27 (1H, m), 2.17-0.64 (20H, m), 0.88 (9H, s), 0.71 (3H, s), 0.07 (3H, s), 0.06 (3H, s).

To a stirred solution of compound g1 (300 mg, 0.68 mmol) in DCM (14 mL), a suspension of PCC (1.03 g, 4.79 mmol) in DCM (4 mL) was added. The reaction mixture was stirred for 2 days, then filtered through a celite pad and washed with DCM. The filtrate was washed with brine, and the organic layer was dried over anhydrous Na₂SO₄, was filtered and the filtrate was evaporated in vacuo. The residue was purified by flash column chromatography (elution solvent chloroform/MeOH 99/1) to yield (5S,10S,13S)-10-((tert-butyldimethylsilyloxy)methyl)-13-methyl-dodecahydro-2H-cyclopenta[a]phenanthrene-3,6,17(14H)-trione (compound g2) as a white solid (280 mg; 95% yield). ¹H-NMR (300 MHz, CDCl₃) δ 3.78 (2H, s), 2.62-1.28 (20H, m), 0.91 (3H, s), 0.86 (9H, s), 0.05 (3H, s), 0.04 (3H, s).

¹³C-NMR (75 MHz, CDCl₃) δ 219.6, 210.3, 206.1, 60.8, 54.6, 53.1, 52.2, 47.9, 44.7, 44.5, 37.3, 37.0, 36.9, 35.5, 33.2, 31.3, 25.6, 21.5, 21.2, 18.0, 13.8, −5.9.

To a solution of compound g2 (220 mg, 0.51 mmol) in THF (3.4 mL) was added a solution of 3-(R)-pyrrolidinyloxyamine dihydrochloride (Intermediate I; 89 mg, 0.51 mmol) in water (1.5 mL). After stirring for 2 h at room temperature NaCl (193 mg, 3.31 mmol) was added, and the reaction mixture was stirred for an additional 15 minutes. The two layers were separated. The aqueous phase was extracted again with THF (×2), and the combined organic phases were dried over anhydrous Na₂SO₄, were filtered and the filtrate was evaporated in vacuo to afford (5S,10S,13S)-10-((tert-butyldimethylsilyloxy)methyl)-13-methyl-3-((S)-pyrrolidin-3-yloxyimino)-tetradecahydro-14H-cyclopenta[a]phenanthrene-6,17-dione (compound g3) (130 mg), which was carried into the next step without any further purification.

To a solution of compound g3 (130 mg, 0.25 mmol) in dry THF (7 mL) was added a solution of TBAF 1M in THF (1.76 mL, 1.76 mmol) and the reaction was stirred overnight at room temperature. After the reaction was completed, MeOH (7 mL), CaCO₃ (350 mg, 3.50 mmol) and DOWEX marathon C (Na⁺ form) were added. The resulting mixture was stirred for 15 min, filtered through a celite pad and washed with MeOH. The filtrate was evaporated under reduced pressure and the residue is purified by column chromatography (elution solvent CHCl₃/MeOH/NH₃ 95/5/1→90/10/1) to afford (5S,10S,13S)-10-(hydroxymethyl)-13-methyl-3-((S)-pyrrolidin-3-yloxyimino)-tetradecahydro-14H-cyclopenta[a]phenanthrene-6,17-dione (Compound 28) as a white solid (70 mg; 69% combined yield from compound g2). ¹H-NMR (600 MHz, CDCl₃) δ 4.69 (1H, s), 4.08 (1H, d, J=8.5 Hz), 3.74 (1H, d, J=8.5 Hz), 3.23-2.81 (7H, m), 2.48-1.01 (18H, m), 0.97 and 0.93 (3H, s).

To a solution of compound 28 (70 mg) in MeOH (0.1 mL), and an equimolar amount of oxalic acid was added. The corresponding oxalate salt of compound 28 was precipitated by the addition of diethyl ether, filtered, and dried. ¹H-NMR (600 MHz, DMSO-d₆) δ 4.76 (1H, s), 4.02 (1H, d, J=8.7 Hz), 3.73 (1H, d, J=8.7 Hz), 3.31-3.09 (6H, m), 2.43-1.01 (18H, m), 0.87 (3H, s).

### Example 8

### Synthesis of the Oxalate Salt of Compound 30: (5S,10S,13S)-10-(hydroxymethyl)-6-methoxy-imino-13-methyl-3-((S)-pyrrolidin-3-yloxyimino)-tetradecahydro-14H-cyclopenta[a]phenanthrene-17-one

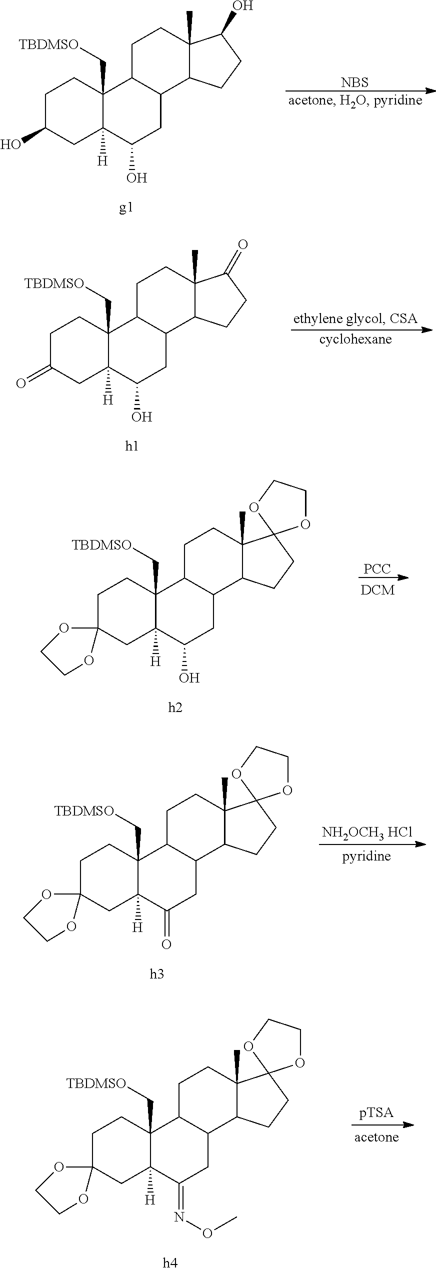

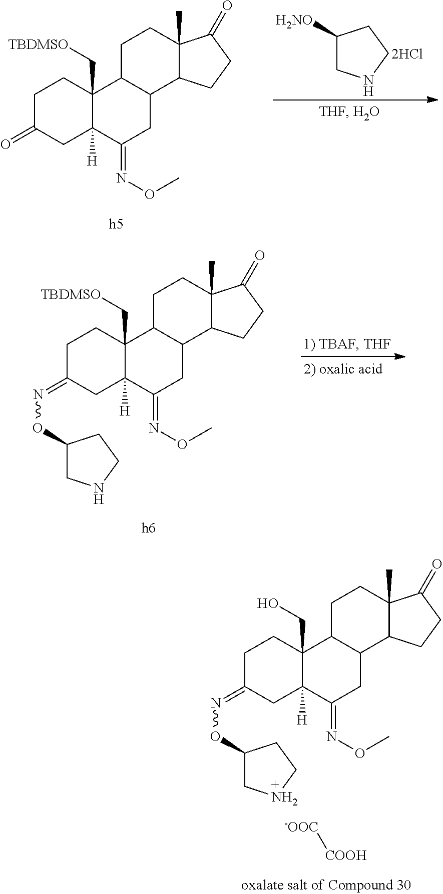

To a stirred solution of compound g1 (350 mg, 0.80 mmol) in a mixture of acetone (24 mL)/water (5.2 mL), were sequentially added pyridine (1.3 mL) and NBS (570 mg, 3.20 mmol), and the reaction mixture was stirred overnight at room temperature. Addition of aqueous HCl (10%, ˜3 mL) was followed by the addition of NaOH until pH 8. The reaction mixture was evaporated and the residue was extracted with ethyl acetate (×3). The combined organic layers were dried over anhydrous Na₂SO₄, were filtered and evaporated to afford 10-(tert-Butyl-dimethyl-silanyloxymethyl)-6-hydroxy-13-methyl-tetradecahydro-cyclopenta[a]phenanthrene-3,17-dione (compound h1), which was carried into the next step without any further purification.

To a stirred solution of crude h1 (350 mg, 0.80 mmol) in cyclohexane (13 mL), ethylene glycol (2.0 mL, 29.0 mmol) and CSA (5.6 mg, 0.024 mmol) were added and the reaction mixture was refluxed for 12 h using a Dean Stark apparatus. The reaction mixture was cooled to room temperature, followed by the addition of Et₂O. The organic phase was extracted with aq. NaHCO₃ and brine, dried over anhydrous Na₂SO₄, filtered and evaporated to afford 10-(tert-butyl-dimethyl-silanyloxymethyl)-6-hydroxy-13-methyl-tetradecahydro-cyclopenta[a]phenanthrene-3,17-diethylene ketal (compound h2), which was carried into the next step without any further purification.

To a stirred solution of crude h2 (420 mg, 0.80 mmol) in DCM (3 mL), a suspension of PCC (340 mg, 1.58 mmol) in DCM (5 mL) was added and the reaction mixture was stirred for 1.5 h. The mixture was filtered through a celite pad, and washed with DCM. The filtrate was extracted with brine, and the organic layer was dried over anhydrous Na₂SO₄, filtered and evaporated in vacuo. The residue was purified by flash column chromatography (elution solvent chloroform/MeOH 95/5) to afford 10-(tert-butyl-dimethyl-silanyloxymethyl)-6-oxo-13-methyl-tetradecahydro-cyclopenta[a]phenanthrene-3,17-diethylene ketal (compound h3) as a white solid (180 mg; 43% combined yield from compound hi). ¹H-NMR (300 MHz, CDCl₃) δ 3.90 (8H, m), 3.63 and 3.59 (2H, AB system, J=10.9 Hz), 2.39-1.12 (20H, m), 0.86 (9H, s), 0.84 (3H, s), 0.02 (3H, s), 0.00 (3H, s).

To a solution of compound h3 (70 mg, 0.134 mmol) in dry pyridine (1.5 mL) NH₂OCH₃.HCl (20 mg, 0.24 mmol) was added at room temperature and the reaction mixture was stirred overnight. Pyridine was evaporated under reduced pressure and to the residue were added DCM (5 mL) and water (5 mL), and the layers are separated. The aqueous layer was washed with DCM (×2) and the combined organic layers were dried over anhydrous Na₂SO₄, filtered and evaporated to afford 10-(tert-butyl-dimethyl-silanyloxymethyl)-6-methoxy-imino-13-methyl-tetradecahydro-cyclopenta[a]phenanthrene-3,17-diethylene ketal (compound h4), which was carried into the next step without any further purification.

To a solution of crude h4 in acetone (5 mL), pTSA (127 mg, 0.67 mmol) was added and the resulting mixture was stirred at room temperature overnight. The reaction mixture was neutralized by addition of 5% aq. NaHCO₃, and the organic phase was separated. The aqueous layer was extracted with DCM (×3) and the combined organic layers were washed with brine, were dried over anhydrous Na₂SO₄, were filtered, and evaporated in vacuo. The residue was purified by flash column chromatography (elution solvent, petroleum ether/diethyl ether 6/4) to afford 10-(tert-butyl-dimethyl-silanyloxymethyl)-6-methoxy-imino-13-methyl-tetradecahydro-cyclopenta[a]phenanthrene-3,17-dione (compound h5) as a white solid (21 mg, 34% combined yield from compound h3). ¹H-NMR (600 MHz, CDCl₃) δ 3.82 (3H, s), 3.81 and 3.76 (2H, AB system, J=11.1 Hz), 3.38 (1H, dd, J=4.8, 14.1 Hz), 2.74 (1H, dd, J=13.8, 16.1 Hz), 2.50-1.06 (18H, m), 0.89 (12H, s), 0.07 (6H, s).

To a solution of compound h5 (21 mg, 0.045 mmol) in THF (1.0 mL) was added a solution of 3-(R)-pyrrolidinyloxyamine dihydrochloride (Intermediate I; 9 mg, 0.05 mmol) in water (0.4 mL). After stirring at room temperature for 2 h, NaCl (18 mg) was added, and the reaction mixture was stirred for an additional 15 min. The two layers were separated and the aqueous layer was extracted with THF (×2) and the combined organic phases were dried over anhydrous Na₂SO₄, were filtered and evaporated in vacuo to afford 10-(tert-butyl-dimethyl-silanyloxymethyl)-6-methoxy-imino-13-methyl-3-((S)-pyrrolidin-3-yloxyimino)-tetradecahydro-cyclopenta[a]phenanthrene-17-one (compound h6) (25 mg), which was carried into the next step without any further purification.

To a solution of compound h6 (25 mg, 0.045 mmol) in dry THF (3 mL), TBAF (1M solution in THF) (0.23 mL, 0.23 mmol) was added and the reaction mixture was stirred overnight at room temperature. After completion of the reaction, MeOH (3 mL), CaCO₃ (45 mg, 0.45 mmol) and DOWEX marathon C (Na⁺ form) were added and the resulting mixture was stirred for 15 min, was filtered through a celite pad and was washed with MeOH. The filtrate was evaporated under reduced pressure and the residue was purified by flash column chromatography (elution solvent CHCl₃/MeOH/NH₃ 95/5/1→90/10/1) to afford (5S,10S,13S)-10-(hydroxymethyl)-6-methoxy-imino-13-methyl-3-((S)-pyrrolidin-3-yloxyimino)-tetradecahydro-14H-cyclopenta[a]phenanthrene-17-one (compound 30) as a white solid (12 mg; 62% combined yield from compound h4): ¹H-NMR (600 MHz, CDCl₃) δ 4.70 (1H, s), 3.84 (3H, s), 3.84 and 3.77 (2H, AB system, J=11.0 Hz), 3.42-2.87 (7H, m), 2.52-1.00 (18H, m), 0.91 (3H, s).

To a solution of compound 30 (12 mg) in MeOH (0.1 mL) an equimolar amount of oxalic acid was added. The corresponding oxalate salt of compound 30 was precipitated by the addition of diethyl ether, filtered, and dried. ¹H-NMR (600 MHz, CD₃OD) δ 4.83 (1H, s), 3.77 (3H, s), 3.66 (2H, AB, J=11.0 Hz), 3.47-3.12 (7H, m), 2.49-1.00 (18H, m), 0.89 (3H, s).

### Example 9

### Synthesis of the Oxalate Salt of Compound 48: ((5S,10R,13S)-13-methyl-17-oxo-3-((R)-pyrrolidin-3-yloxyimino)-hexadecahydro-1H-cyclopenta[a]phenanthren-10-yl)methyl acetate

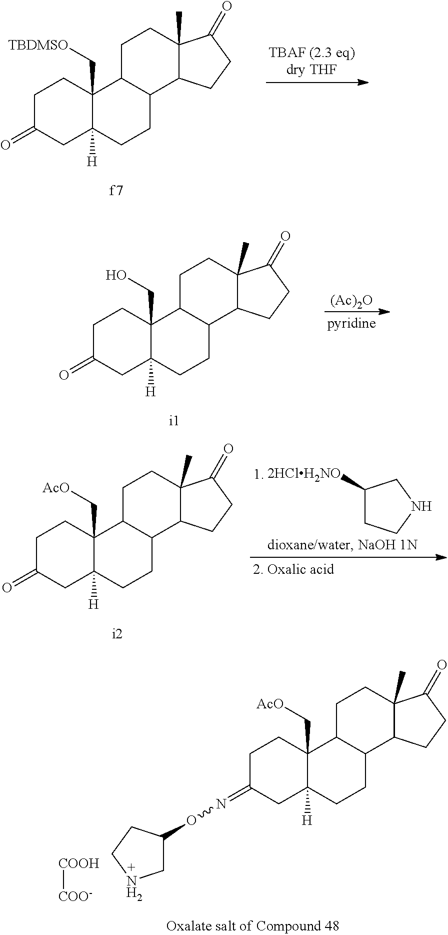

To a solution of compound f7 (100 mg) in dry THF (3 mL), under argon, TBAF (1M in THF, 0.55 mL) was added and the mixture was stirred overnight. The organic solvent was evaporated and the crude product was diluted with dichloromethane (10 mL) and washed with H₂O and brine. The combined organic layers were dried over anhydrous sodium sulfate and evaporated to dryness under reduced pressure. The residue was purified by flash column chromatography (SiO₂, cyclohexane/ethyl acetate 1/1) to afford 61 mg (88) of compound i1 as a colourless solid. ¹H-NMR (300 MHz, CDCl₃) δ 4.24-3.91 (m, 2H), 2.52-0.83 (m, 26H).

Acetic anhydride (28 L) was added at 0° C. to a solution of compound i1 (60 mg) in anhydrous pyridine (0.2 mL) under argon. The mixture was stirred overnight at room temperature, then it was carefully diluted with H₂O (5 mL) and extracted with dichloromethane (3×10 mL). The combined organic layers were dried over anhydrous sodium sulfate and evaporated to dryness under reduced pressure. The crude product was purified by flash column chromatography (SiO₂, cyclohexane/ethyl acetate 7/3) to afford 65 mg (95%) of compound i2 as a colourless solid. ¹H-NMR (300 MHz, CDCl₃) δ 4.50 and 4.40 (AB system J=12.3 Hz, 2H), 2.51-0.88 (m, 22H), 2.10 (s, 3H), 0.88 (s, 3H).

A solution of dioxane/H₂O (2/1, 7.5 mL) was treated with NaOH 1N to pH=12 and compound i2 (65 mg) was added. Then, a solution of 3-(R)-pyrrolidinyloxyamine dihydrochloride (Intermediate I; 35 mg) in H₂O (1 mL) was added. After 1.5 h, the organic solvent was distilled off and the mixture was diluted with water and extracted with chloroform (3×20 mL). The combined organic layers were dried over anhydrous sodium sulfate and evaporated to dryness under reduced pressure. The residue was purified by flash chromatography (SiO₂, chloroform/methanol/ammonia 95/5/0.5) to afford 40 mg compound 48, which was dissolved in ethyl acetate/methanol 8/2 and treated with oxalic acid (11 mg) to give 18 mg of the corresponding oxalate salt as a white solid. ¹H-NMR (600 MHz, CD₃OD) δ 4.83 (m, 1H), 4.52 (dd, J₁=12.0, 4.8 Hz, 1H), 4.37 (m, 1H), 3.56-3.53 (m, 1H), 3.42-3.35 (m, 3H), 3.18-3.15 (m, 0.5H), 3.01-2.98 (m, 0.5H), 2.47-0.88 (m, 26H), 2.06 (s, 3H), 0.88 (s, 3H). ¹³C-NMR (75 MHz, CD₃OD) δ 223.7, 172.8, 167.6, 162.6, 162.5, 81.3, 63.2, 55.4, 52.9, 51.4, 48.0, 46.3, 45.3, 40.2, 40.1, 36.7, 35.4, 34.6, 33.8, 33.1, 31.7, 31.2, 29.3, 29.2, 28.7, 22.6, 22.5, 21.1, 14.3.

### Example 10

### Synthesis of the Hydrochloride Salt of Compound 59: (11-Fluoro-10,13-dimethyl-6-methylene-tetradecahydro-cyclopenta[a]phenanthrene-3,17-dione 3-(O-pyrrolidin-3-yl-oxime))

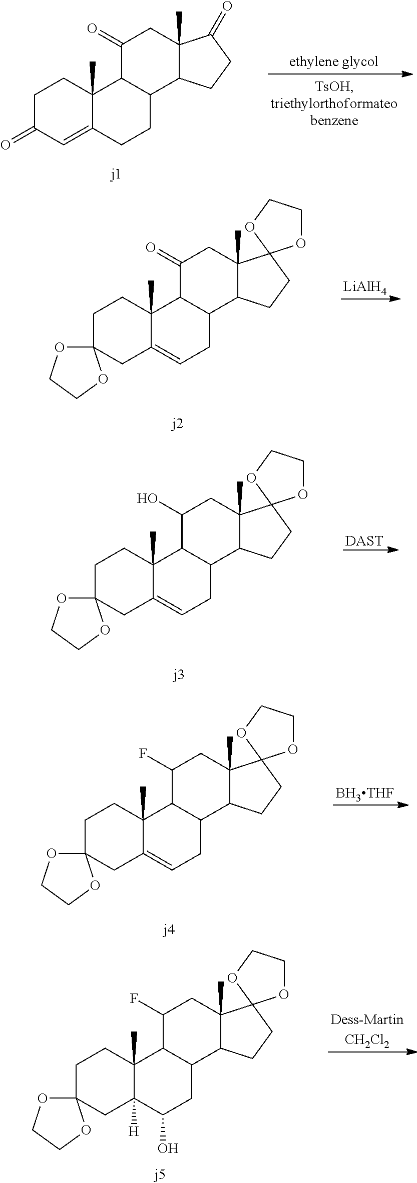

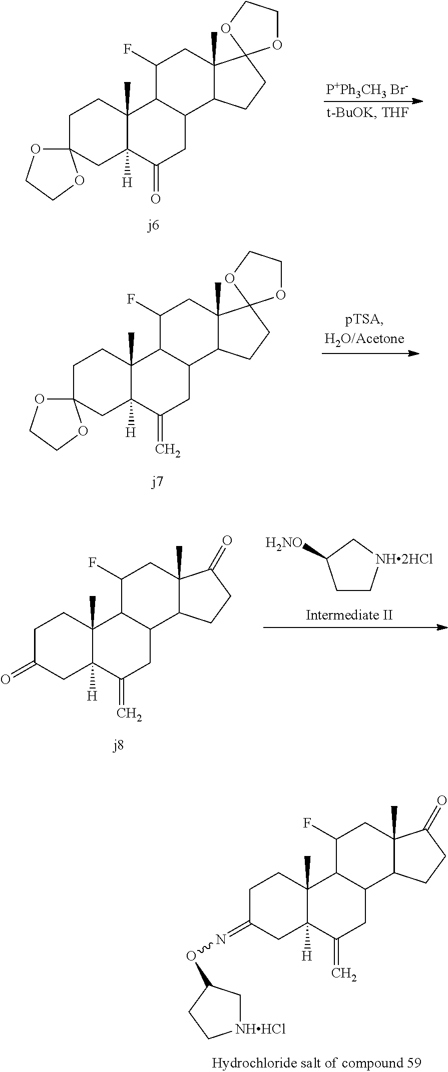

Andrenosterone (Zhao Q, Li Z., Steroids 1994, 59 190-195), upon reaction with ethylene glycol and trimethylorthoformate in benzene in the presence of TsOH can yield the ethylene diketal j2, which can react with a variety of reducing agents (i.e. LiAlH₄) to afford the 11-hydroxy derivative j3, which can react with nucleophilic fluorinating reagents (i.e. DAST) to give the corresponding 11-fluoro compound j4. Treatment of analogue j4 with borane.tetrahydro furan complex can yield mainly the 6α-hydroxy compound j5, which can be further oxidized to the 6-keto derivative j6. Wittig reaction of j6 with methyltriphenyl phosphonium bromide can afford the 6-methylene derivative j7. Removal of the protecting group at C3 with TsOH in acetone/water can give the 3-keto derivative j8 which can react with Intermediate II to give compound 59 as the free base or the salt.

### Example 11

### Synthesis of the Hydrochloride Salt of Compound 62: (12-Fluoro-10,13-dimethyl-6-methylene-tetradecahydro-cyclopenta[a]phenanthrene-3,11,17-trione 3-(O-pyrrolidin-3-yl-oxime))

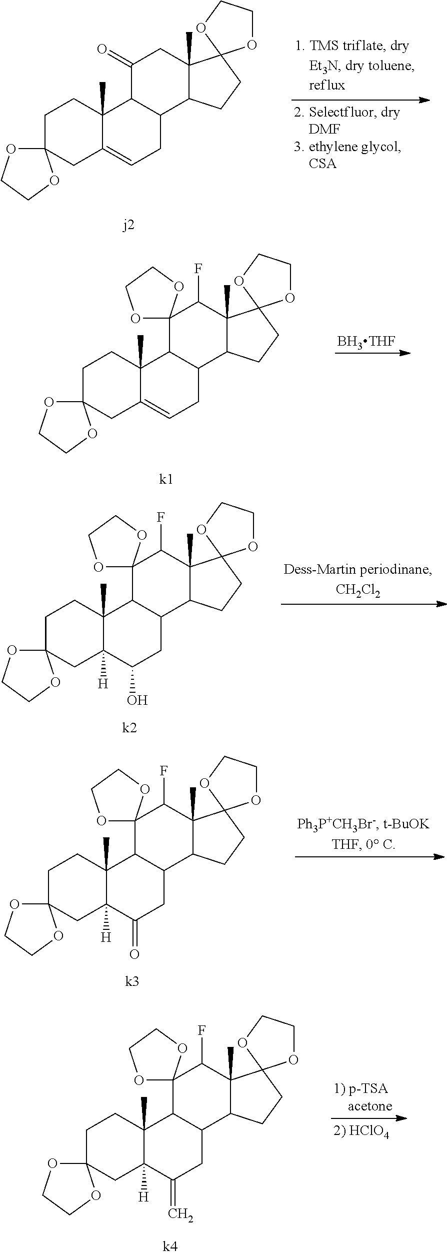 [SMILES: C[C@]12CCC3(CC1=CCC1C2C(=O)C[C@@]2(C)C1CCC21OCCO1)OCCO3 ; C[C@]12C(CCC13OCCO3)C1CC=C3CC4(CC[C@]3(C)C1C1(OCCO1)C2F)OCCO4 ; C[C@]12C(CCC13OCCO3)C1C[C@H](O)[C@H]3CC4(CC[C@]3(C)C1C1(OCCO1)C2F)OCCO4 ; "BH3·THF" ; C[C@]12C(CCC13OCCO3)C1CC(=O)[C@H]3CC4(CC[C@]3(C)C1C1(OCCO1)C2F)OCCO4 ; C=C1CC2C3CCC4(OCCO4)[C@@]3(C)C(F)C3(OCCO3)C2[C@@]2(C)CCC3(C[C@H]12)OCCO3]

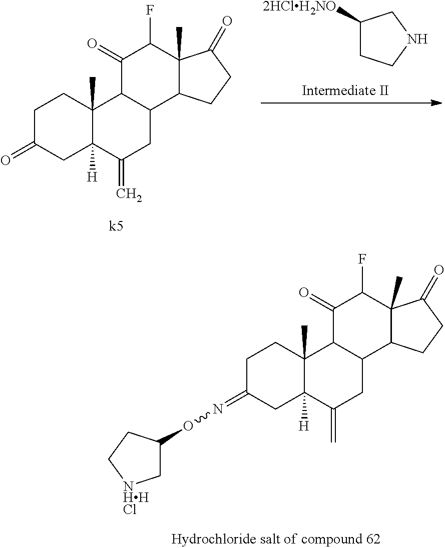

The ethylene diketal j2 upon reaction with TMS triflate in the presence of triethylamine can yield the corresponding TMS enol ether which upon treatment with electrophilic fluorinating reagents (i.e. Selectfluor) can afford the corresponding 12-fluoro derivative, which upon treatment with ethylene glycol in the presence of acid (i.e camphor sulfonic acid) can yield the ethylene triketal k1. Treatment of k1 with borane.tetrahydrofuran complex can yield mainly the 6α-hydroxy compound k2, which can be further oxidized with a number of oxidants (i.e. Dess Martin periodinane) to the 6-keto derivative k3. Wittig reaction of k3 with methyl triphenyl phosphonium bromide can afford the 6-methylene derivative k4. Removal of the protecting group at C3 and C17 with TsOH in acetone/water, followed by treatment with perchloric acid at low temperature can give the triketone k5 which can react with Intermediate II to give compound 62 as the free base or the salt.

### Example 12

### Synthesis of the Hydrochloride Salt of Compound 65: (12,16-Difluoro-10,13-dimethyl-6-methylene-tetradecahydro-cyclopenta[a]phenanthrene-3,11,17-trione 3-(O-pyrrolidin-3-yl-oxime))

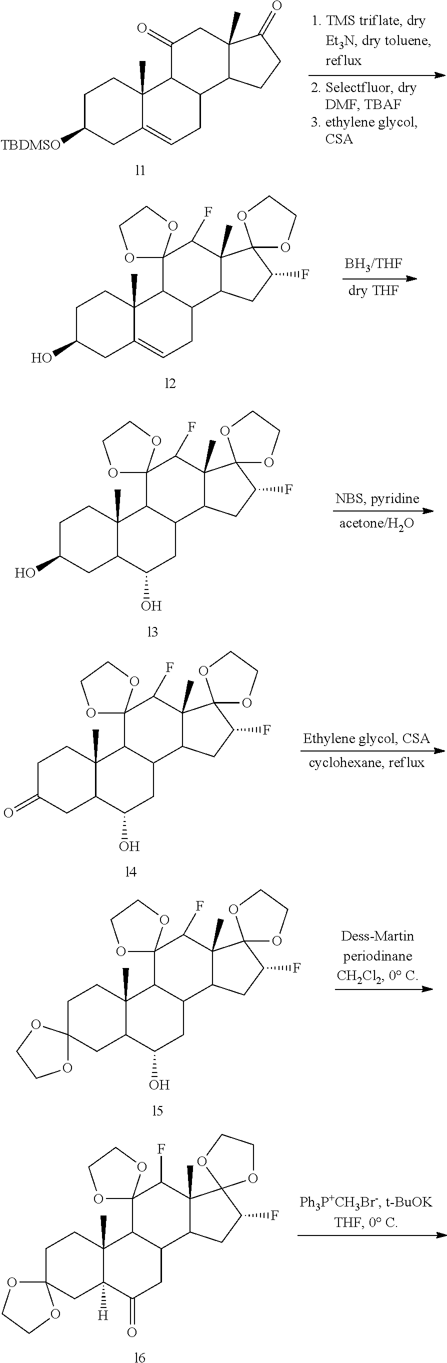

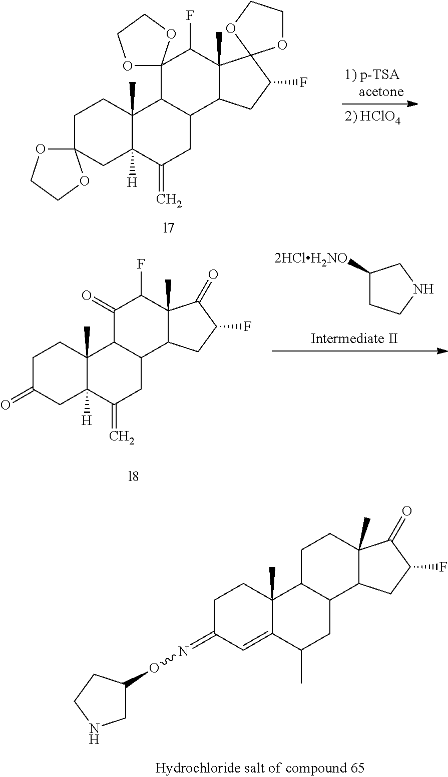

Compound I1 (Moon, S., Stuhmiller, L. M.; Chadha, R. K., McMorris, T. C., Tetrahedron 1990, 46(7), 2287-2306) upon reaction with TMS triflate in the presence of triethylamine can yield the corresponding TMS enol ether which upon treatment with electrophilic fluorinating reagents (i.e. Selectfluor) can afford the corresponding 12,16-difluoro derivative, which upon treatment with ethylene glycol in the presence of acid (i.e camphor sulfonic acid) can yield the ethylene diketal l2. Treatment of l2 with borane.tetrahydrofuran complex can yield mainly the 6α-hydroxy compound l3, which can be oxidized at C3 by treatment with NBS/pyridine in acetone/water to give the 3-keto analogue l4, which upon treatment with ethylene glycol in the presence of acid (i.e camphor sulfonic acid) can yield the ethylene triketal l5. Oxidation of the 6-hydroxy group in l5 can be achieved by a number of oxidants (i.e. Dess Martin periodinane) to the 6-keto derivative l6. Wittig reaction of l6 with methyl triphenylphosphonium bromide can afford the 6-methylene derivative l7. Removal of the protecting group at C3 with TsOH in acetone/water, followed by treatment with perchloric acid at low temperature can give the triketone l8 which can react with Intermediate II to give compound 62 as the free base or the salt.

### Example 13

### Synthesis of the Hydrochloride Salt of Compound 68: (15-Fluoro-10,13-dimethyl-6-methylene-tetradecahydro-cyclopenta[a]phenanthrene-3,17-dione 3-(O-pyrrolidin-3-yl-oxime))

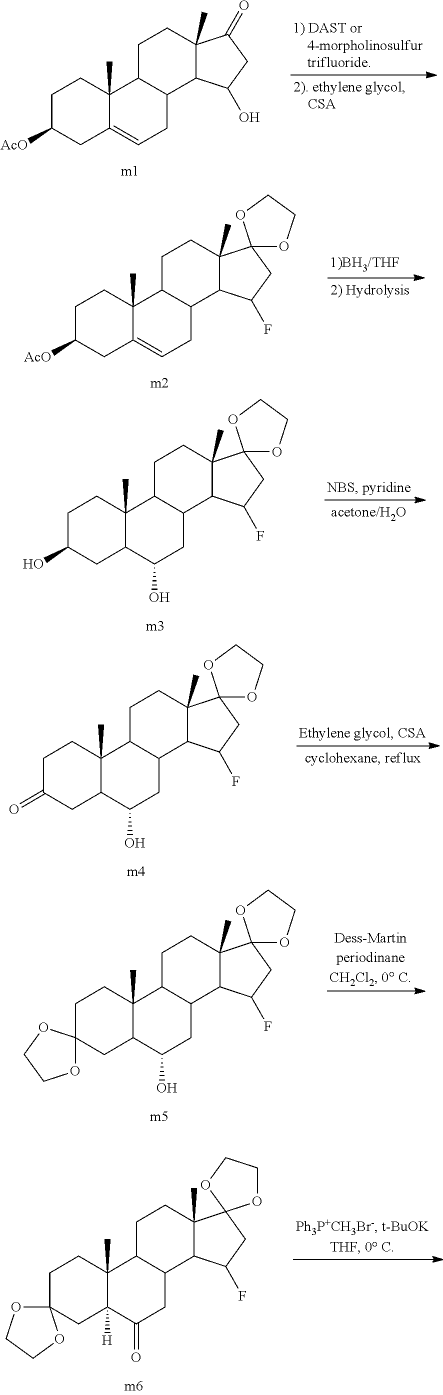

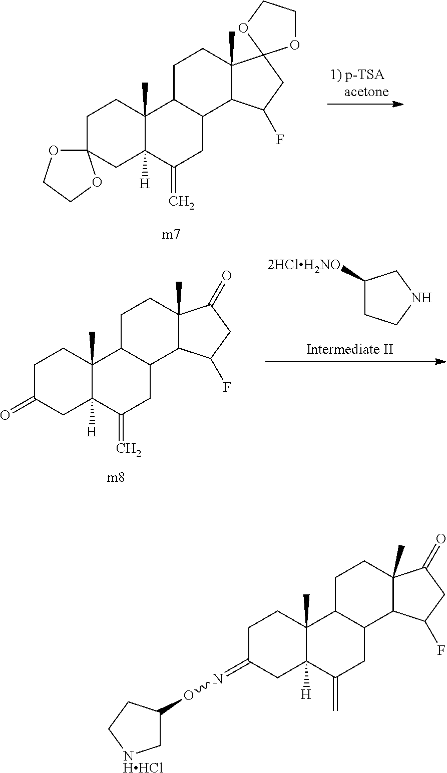

### Hydrochloride Salt of Compound 68

Compound m1 (Cerny, I., Fajkos, J., Pouzar, V. Steroids 1996, 61:58-64) upon reaction with a nucleophilic fluorinating reagent such as DAST or morpholino sulfur trifluoride can afford the corresponding 15-fluoro analogue which upon reaction with ethylene glycol in the presence of acid (i.e camphor sulfonic acid) can yield the ethylene ketal m2. Treatment of m2 with borane.tetrahydrofuran complex can yield mainly the 6α-hydroxy compound m3 after hydrolysis, which can be oxidized at C3 by treatment with NBS/pyridine in acetone/water to give the 3-keto analogue m4, which upon treatment with ethylene glycol in the presence of acid (i.e camphor sulfonic acid) can yield the ethylene diketal m5. Oxidation of the 6-hydroxy group in m5 can be achieved by a number of oxidants (i.e. Dess Martin periodinane) to the 6-keto derivative m6. Wittig reaction of m6 with methyl triphenylphosphonium bromide can afford the 6-methylene derivative m7. Removal of the protecting groupw at C3 and C17 with acid, such as TsOH, in acetone/water, can give the diketone m8 which can react with Intermediate II to give compound 68 as the free base or the salt.

### Example 14

### Synthesis of the Oxalate Salt of Compound 71: (16-Fluoro-10,13-dimethyl-6-methylene-tetradecahydro-cyclopenta[a]phenanthrene-3,17-dione 3-[O-(2-piperidin-1-yl-ethyl)-oxime])

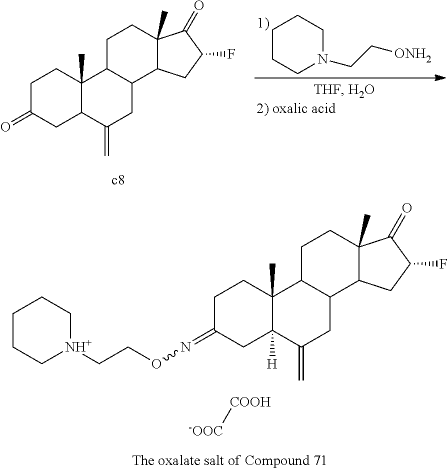

Reaction of c8 with O-(2-(piperidin-1-yl)ethyl)hydroxylamine (Favara, D., Nicola, M., Pappalardo, M., Bonardi, G., Luca, C., Marchini, F., Sardi B. Farmaco, Edizione Scientifica, 1987, vol. 42 (10), 697-708) to give compound 71 as the free base or the salt after treatment with acid, such as oxalic acid.

### Example 15

### Synthesis of the Oxalate Salt of Compound 86: (16-Fluoro-6,10,13-trimethyl-1,6,7,8,9,10,11,12,13,14,15,16-dodecahydro-2H-cyclopenta[a]phenanthrene-3,17-dione

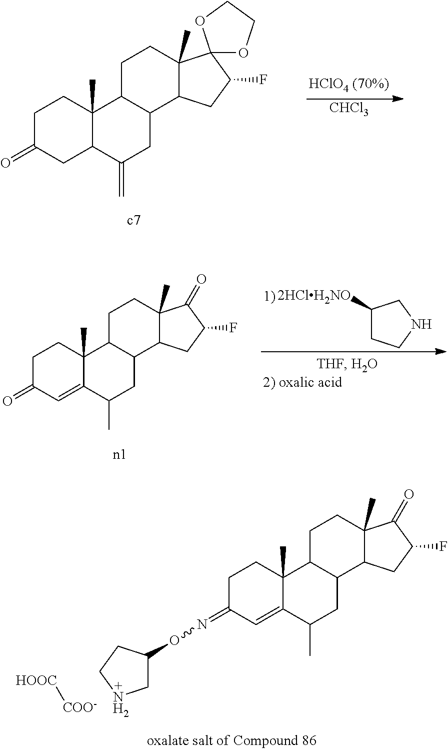

Perchloric acid 70% (0.4 mL) was added at 0° C. to a solution of c7 (80 mg, 0.221 mmol) in chloroform (10 mL), the mixture was stirred at room temperature for 0.5 h and saturated NaHCO₃ was added. The crude product was extracted with chloroform (3×10 mL) and the combined organic layers were dried over anhydrous sodium sulfate and evaporated to dryness under reduced pressure. The residue was purified by flash column chromatography (SiO₂, petroleum ether/ethyl acetate 9/1) to afford 34 mg (50%) of compound n1 as a white solid. ¹H-NMR (600 MHz, CDCl₃) δ 5.81 (s, 1H), 5.09 (1H, dd, J=50.4, 7.2 Hz), 2.50-2.31 (m, 3H), 2.15-0.91 (m, 16H), 1.21 (s, 3H), 1.10 (d, J=6 Hz), 0.97 (s, 3H); ¹⁹F-NMR (282.1 MHz, CDCl₃) δ −192.4 (m).

To a stirred solution of n1 (37 mg, 0.116 mmol) in THF (3.0 mL) was added dropwise a solution of 3-(R)-pyrrolidinyloxyamine dihydrochloride (22 mg, 0.128 mmol) in water (1.2 mL). After 45 min NaCl (44 mg, 0.754 mmol) was added, the reaction mixture was stirred for an additional 15 min and extracted. The aqueous phase was washed with THF (×2) and the combined organic phases were dried over Na₂SO₄, filtered and evaporated under reduced pressure and the residue was purified by flash column chromatography (elution solvent chloroform/methanol/ammonia 90/10/1) to afford Compound 86, 23 mg (50%) as white solid. ¹H-NMR (300 MHz, CDCl₃) δ 6.38 and 5.84 (bs, 1H), 5.57 and 5.09 (dd, J=50.4, 4.5 Hz, 1H), 4.79-4.73 (m, 2H), 4.80-4.70 (m, 1H), 3.22-2.71 (5H, m), 2.31-0.82 (16H, m), 1.08 (d, J=6.38, 3H), 1.06 (s, 3H), 0.92 (s, 3H).

Compound 86 was dissolved in MeOH (0.1 mL), an equimolar amount of oxalic acid was added and the corresponding salt was precipitated by the addition of diethyl ether, was filtered and dried.

### II. BIOLOGICAL EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques may be considered in making and practicing the invention. Particularly useful techniques for particular embodiments will be discussed in the sections that follow.

A. Material and Source thereof.

### Reagents

Testosterone-HSA (testosterone 3-(O-carboxymethyl)oxime: human serum albumin) conjugates and fluorescein isothiocyanate (FITC) were purchased from Sigma (St. Louis, Mo.).

### Synthesis of Fluorescent Testosterone-HSA-FITC (TAC) and HSA-FITC Conjugates

To label testosterone-HSA or HSA conjugates with FITC, a freshly prepared solution containing 6 mg of fluorescein isothiocyanate in 3 ml of 0.1M sodium carbonate buffer at pH 9.5 and 0° C. was added with stirring to 60 mg of a testosterone-human serum albumin conjugate (HSA) or HSA dissolved in 12 ml buffer and stirring was continued overnight at 4° C. The reaction mixture was placed in a dialysis tubing and dialyzed for five days against 2 liters of 10 mM ammonium bicarbonate NH₄HCO₃ at 4° C. Fresh dialysis solution was provided at 24 hour intervals. After dialysis, the sample was examined by thin layer chromatography to preclude the existence of free un-reacted testosterone or FITC and was subsequently lyophilized to dryness yielding 40.2 mg of Testosterone-HSA-FITC and 39.2 mg of HSA-FITC conjugate respectively. To determine the relative conjugation ratios of HSA to FITC in each case, 0.3 mg of Testosterone-HSA-FITC, HSA-FITC conjugate or FITC were dissolved in 2 ml of 0.05 M Tris buffer pH=8.4 and the absorbance at 252, 280 and 495 nm was measured. Based on the spectrophotometer results, testosterone-HSA-FITC conjugates contained 8.7 moles FITC per mole HSA, whereas HSA-FITC conjugates contained 7.62 moles FITC per mole HSA.

### Cell Lines

All cell lines were obtained from the American Type Culture Collection (Manassas, Va.) or the National Cancer Institute, NIH (Bethesda, Md., USA) and were adapted to grow in the commercially available culture media RPMI 1640, which was supplemented with 25 mM HEPES, 2 mM L-Glutamine, 5-10% fetal bovine serum and antibiotics in a 5% CO₂ humidified atmosphere (100%) at 37° C.

### B. Assays and Results Thereof

Examples of the biological experiments as performed are described in this section, the examples, and the relevant literature, which in no way should be construed as being further limiting.

### Example 16

### Na⁺ K-ATPase Inhibitory Activity of the Compounds of the Invention

The compound inhibitory effect on ATPase activity was assessed in vitro using the colorimetric Adenosine 5′-Triphosphatase Enzymatic Assay of Sigma (St. Louis, Mo.) according to the manufacturer's instructions. This assay utilizes Adenosine 5′-Triphosphatase isolated from porcine cerebral cortex. Each reaction was performed in a final volume of 2501 using 0.5 units/ml enzyme in the presence of the different inhibitors. For the determination of the IC₅₀ values of each compound, each inhibitor was added in five concentrations ranging from 10⁻⁵ to 10⁻⁹M (in triplicates). IC₅₀ values were determined using the Origin® program software (OriginLab, Northampton, Mass.).

The Na⁺/K⁺-ATPase inhibitory activities of Compound Nos. 2, 4, 8, 21, 25, 28, and 48 are summarized in Table 3.

TABLE 3
Na+/K+-ATPase inhibitory activity of tested compounds

| Compound | Na+ K+ Inhibitory Activity (IC₅₀, μM) |
|---|---|
| Comp 2 | 0.073 |
| Comp 4 | 0.194 |
| Comp 8 | 0.311 |
| Comp 21 | 19.3 |
| Comp 25 | 0.525 |
| Comp 28 | 12.9 |
| Comp 30 | 7.54 |
| Comp 48 | 2.88 |

As shown in Table 3, all compounds being tested inhibited the activity of the Na⁺K⁺ ATPase. Among them, compounds 2, 4, and 8 were very potent Na⁺K⁺ ATPase inhibitors and inhibited enzyme activity with an IC₅₀ value below 500 nM. Compound 2 showed the strongest Na⁺K⁺ ATPase inhibitory activity among the compound being tested (with an IC₅₀ value of 73 nM).

### Example 17

### Compounds of the Invention Had Significant Anti-Cancer Activity in Cancer Cell Lines

Sulforhodamine B (SRB) assays were performed according to standard National Cancer Institute Guidelines. Briefly, human tumor cells from different cancer panels were seeded into 96 well plates in 100 L (plating densities ranging from 5-40,000 cells/well depending on the doubling time of individual cell lines) in serum-containing media for twenty-four hours prior to the addition of the compounds to be assayed. After 24 hours, one plate of each cell line was fixed in situ with trichloroacetic acid (TCA). The fixed slides were used to determine the cell population for each cell line at the time of drug addition (Tz). Each compound to be tested was solubilized in dimethyl sulfoxide (DMSO) and the desired concentration was then added to the medium and diluted serially 1:10 to provide a total of five or ten drug concentrations plus control (in a final volume of 200 μL). The starting dose before any dilution was 100 uM for all compounds.

Following drug addition, each culture was incubated for forty-eight hours. The assay was terminated with the addition of cold TCA. The supernatant was discarded, and the plates were washed with tap water and air dried. SRB solution was then added to each well. After staining, the bound stain was solubilized and the absorbance was read on an automated plate reader at a wavelength of 515 nm.

Using the absorbance measurements [time zero, (Tz), control growth, (C), and test growth in the presence of drug at the ten concentration levels (Ti)], the percentage growth was calculated at each of the drug concentrations levels. Percentage growth inhibition was calculated as:

[(Ti−Tz)/(C−Tz)]×100 for concentrations for which Ti>/=Tz

[(Ti−Tz)/Tz]×100 for concentrations for which Ti<Tz.

Three dose response parameters were calculated for each experimental agent. Growth inhibition of 50% (GI50) was calculated from [(Ti−Tz)/(C−Tz)]×100=50, which was the drug concentration resulting in a 50% reduction in the net protein increase (as measured by SRB staining) in control cells during the drug. The drug concentration resulting in total growth inhibition (TGI) was calculated from Ti=Tz. The LC50 (concentration of drug resulting in a 50% reduction in the measured protein at the end of the drug treatment as compared to that at the beginning) indicating a net loss of cells following treatment was calculated from [(Ti−Tz)/Tz]×100=−50.

Values were calculated for each of these three parameters when the level of activity was reached; however, if the effect was not reached or was exceeded, the value for that parameter was expressed as greater or less than the maximum or minimum concentration tested.

SRB provides a colorimetric measure of a compound's anti-cancer activity. SRB assays were performed with compounds of the invention in 7 different cell lines, which represent six different tumor panels (lung, ovarian, prostate, CNS, breast, colon).

Table 4 summarizes results characterizing the effects of compounds 2, 4, 6, 8, 21, 25, 28, 30, 48, and 86 on GI50, which measures the cell growth inhibitory power of the compound, TGI, which measures cytostatic effect and LC50, which signifies cytotoxic effect. Each of these parameters was calculated in a SRB assay.

TABLE 4
In vitro characterization of Na⁺K⁺ ATPase inhibitors in 7 cell lines (SRB)

| Cell Line | | Comp. 2 | Comp. 4 | Comp. 6 | Comp. 8 | Comp. 21 | Comp. 25 | Comp. 28 | Comp. 30 | Comp. 48 | Comp. 86 |
|---|---|---|---|---|---|---|---|---|---|---|---|
| A549 | GI50 | 0.009 | 0.430 | 0.005 | 0.006 | 4,405 | 4,806 | 62,596 | 4,073 | 6,308 | 2,448 |
| Lung | TGI | 0.073 | 0.723 | 0.008 | 0.029 | 8,045 | 9,408 | >100 | 7,357 | 38,323 | 5,248 |
| | LC50 | 0.599 | 1.015 | 0.053 | 0.075 | 45,340 | >100 | >100 | 29,554 | 102,214 | 8,049 |
| H460 | GI50 | <0.001 | nd | 0.003 | 0.004 | nd | 4,374 | 57,566 | nd | nd | 2,572 |
| Lung | TGI | 0.007 | nd | 0.008 | 0.027 | nd | 7,532 | >100 | nd | nd | 5,321 |
| | LC50 | 0.054 | nd | 0.053 | 0.074 | nd | >100 | >100 | nd | nd | 8,071 |
| NCI-ADR-RES | GI50 | 0.029 | nd | 0.018 | 0.014 | nd | nd | nd | nd | nd | 4,132 |
| Ovarian | TGI | 0.094 | nd | 0.049 | 0.071 | nd | nd | nd | nd | nd | 7,963 |
| | LC50 | 0.598 | nd | 0.079 | 0.508 | nd | nd | nd | nd | nd | 48,061 |
| PC3 | GI50 | 0.046 | 0.731 | 0.003 | 0.009 | 4,455 | 3,890 | 53,452 | 7,713 | 8,765 | 5,371 |
| Prostate | TGI | 0.414 | 3.239 | 0.055 | 0.199 | 8,312 | 6,664 | 105,079 | 36,737 | >100 | 9,782 |
| | LC50 | 0.962 | 8.035 | 1.241 | 0.919 | 49,103 | 9,439 | 156,706 | 75,210 | >100 | 72,431 |
| SF268 | GI50 | 0.003 | 0.277 | <0.001 | 0.004 | 4,164 | 5,739 | 63,757 | 5,623 | 11,542 | 1,859 |
| CNS | TGI | 0.043 | 0.782 | 0.020 | 0.038 | 8,450 | 31,254 | >100 | 25,834 | 56,422 | 4,982 |
| | LC50 | 0.112 | 6.976 | 0.071 | 0.085 | 57,216 | 85,428 | >100 | 78,391 | 101,302 | 8,105 |
| MCF7 | GI50 | 0.021 | nd | 0.009 | 0.024 | nd | 6,167 | 76,456 | nd | nd | 4,354 |
| Breast | TGI | 0.478 | nd | 0.082 | 0.327 | nd | 35,207 | >100 | nd | nd | 8,353 |
| | LC50 | 9.783 | nd | 1.285 | 6.965 | nd | >100 | >100 | nd | nd | 59,327 |
| HCT116 | GI50 | 0.008 | nd | 0.009 | 0.035 | nd | nd | nd | nd | nd | 3,710 |
| Colon | TGI | 0.156 | nd | 0.088 | 0.236 | nd | nd | nd | nd | nd | 8,009 |
| | LC50 | 0.998 | nd | 0.728 | 1.038 | nd | nd | nd | nd | nd | 58,762 |

Activity: μM; nd: not done

The results obtained with SRB assays showed that compounds of the invention have significant anti-cancer activity in multiple cell lines. Some of the compounds (e.g., compounds 21, 25, 30, 48) exhibited single-digit micromolar GI50 action in most cell lines, whereas others (e.g., compounds 2, 4, 6 and 8) had on average GI50 values lower than 1 uM. When comparing LC50 values, compounds 2, 6 and 8 are the most potent with LC50 values below 1 μM. Compounds 6 and 8 had the best activity among all the compounds being tested.

### Example 18

### Compounds of the Invention Inhibited the Proliferation and Survival of Neoplastic Cells

Cell proliferation/viability was assessed by the MTT [3-(4,5-dimethylthiazol-2-yl)-2,5-diphenyltetrazolium bromide] assay, which is commercially available (Sigma, St. Louis, Mo.). Cells were cultured in 96-well plates for twenty-four hours (7-10.000 cells/well) and incubated with various concentrations of the different compounds—as indicated in the Figures—in serum-containing medium (or left untreated) for seventy-two hours. At the end of incubation, the medium was aspirated and MTT dissolved in RPMI 1640 w/o phenol red was added to each well to a final concentration of 0.25 mg/ml. After four hours incubation in the dark (37° C., 5% CO₂) the supernatant was discarded. The converted dye (blue formazan crystals) was solubilized by adding 200 μl dimethylsulfoxide to each well. Absorbance was measured at 550 nm with reference at 655 nm using a spectrophotometer with the respective filters. All assays were performed in triplicate.

The effect of the different compounds on the proliferation and viability of two cancer cell lines (DU145/prostate and CAKI/renal) was tested through quantitative (4,5-dimethylthiazol-2-yl)-2,5-diphenyltetrazolium bromide (MTT) assays. The calculated IC₅₀ results as obtained are presented in Table 5. These results are consistent with the SRB results and demonstrate the in vivo anti-cancer action of the compounds of the invention.

TABLE 5
In vitro characterization of Na⁺K⁺ ATPase inhibitors in
2 cell lines (MTT)

| | DU145 (Prostate) (IC50, | CAKI (Renal) |
|---|---|---|
| Compound | μM) | (IC50, μM) |
| Comp 2 | 0.25 | 8.75 |
| Comp 4 | 2.7 | 5 |
| Comp 6 | nd | nd |
| Comp 8 | 0.223 | nd |
| Comp 21 | 13.5 | nd |
| Comp 25 | 15 | nd |
| Comp 28 | 87 | nd |
| Comp 30 | 6.7 | Nd |
| Comp 48 | 11.6 | 41.3 |

nd: not done

Note that calculated IC₅₀ values based on the MTT assay may differ from GI50 values calculated with the SRB assay due to inherent difference in assay methodologies.

### Example 19

### Effect of the Compounds of the Invention Against Multidrug Resistant Neoplasia

Table 4 shows SRB assay results in NCI-ADR-RES cells, a cell line that expresses high levels of multidrug resistance (MDR) gene MDR1 and P-glycoprotein.

The results obtained in these cells are comparable to the results obtained in other cell lines and confirm that the capacity of compounds of the invention to block growth/viability of MDR cells.

### Example 20

### Compound 2 Binds the Membrane Androgen Receptor

The membrane androgen receptor (mAR) is capable of transmitting rapid (non-genomic) androgen signals resulting in robust actin cytoskeleton re-organization in membrane androgen expressing cells (Papadopoulou et al, IUBMB Life. 2009 January; 61(1):56-61). Although it has been speculated that mAR may comprise a membrane-bound form of the classical, pro-oncogenic intracellular androgen receptor (AR), recent results point to the existence of a novel receptor that is structurally and functionally distinct from the classical androgen receptor (reviewed in Papadopoulou et al, IUBMB Life. 2009 January; 61(1):56-61). For example, mAR-induced effects are observed even in the presence of anti-androgens and/or in cell lines deficient in the classical intracellular androgen receptor. In addition, membrane androgen receptors comprising testosterone-albumin conjugates are capable of inducing cell death—rather than proliferation—in AR-positive as well as AR-deficient cells. Consequently, testosterone-albumin conjugates have been shown to possess significant anti-cancer activity in various in vitro and in vivo models, independent of the status of the classical androgen receptor (reviewed in Papadopoulou et al, IUBMB Life. 2009 January; 61(1):56-61, see also PCT/IB03/02785). Similar results have been reported for dihydro-testosterone bovine serum albumin (DHT-BSA) conjugates in C6 cells (Gatson et al, Endocrinology 2006, 147:2028-2034).

The detection of membrane androgen receptors using fluorescent testosterone-serum albumin conjugates was performed as described by Kampa, M et al., 2002, Faseb J 16:1429-1431, with minor modifications. The assay was used to measure the ability of a given compound to preclude binding of a fluorescent mAR ligand (testosterone-HSA-FITC conjugates) to its receptor in DU145 cells; this compound-induced effect is indicative of direct mAR binding (see Kampa et al and PCT/US2007/007913). Briefly, DU145 prostate cancer cells were cultured on 0.1% gelatin-coated glass coverslips in RPMI 1640 medium supplemented with 2 mM L-Glutamine, 10% Fetal Bovine Serum and 1% Penicillin/Streptomycin for at least 48 hours until reaching 70% confluency. After a 24 hour serum starvation period, cells were incubated with 40 M testosterone-HSA-FITC for 1 hour at room temperature. The cells were then washed three times with phosphate buffered saline (PBS) and fixed with 3.7% formaldehyde in PBS for 5 minutes at room temperature. Human serum albumin-FITC was used as a control for background staining. After permeabilization with ice-cold acetone for 4 minutes at room temperature, cell nuclei were stained with DAPI. Coverslips were mounted on slides by using the Slow Fade/Antifade Reagent (Molecular Probes) and studied under a LEICA DMLB microscope, equipped with the appropriate fluorescence filters and a Leica DC 300F camera. Specimens were analyzed using the Leica FW4000 computer program.

Membrane androgen receptor competition assays were performed to determine the capacity of a given compound to preclude binding of fluorescent testosterone-HSA conjugates (TAC) to the membrane androgen receptor. Starved DU145 cells on coverslips were pre-treated with 40 M of the indicated compound for 30 minutes prior to the addition of testosterone-HSA-FITC. At the end of the incubation period, cells were washed twice with PBS and 40 M of testosterone-HSA-FITC was added. Specimens were prepared and analyzed as described above. Compounds binding to the membrane androgen receptor preclude binding of fluorescent testosterone-HSA conjugates to their target and abolish membrane-specific fluorescence.

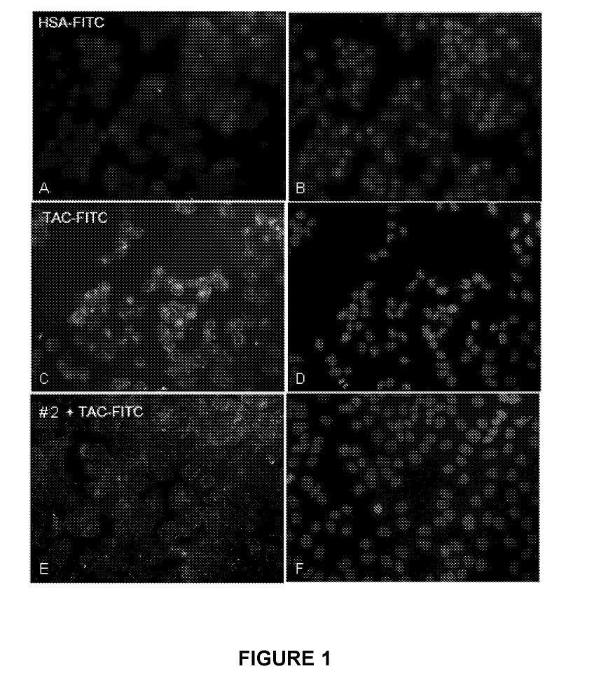

FIG. 1 presents data with compound 2 in DU145 cells. As can be seen from the results, Testosterone-human serum albumin (HSA)-FITC (TAC-FITC) conjugates showed clear membrane staining as compared to control HSA-FITC conjugates (FIG. 1: compare panels C and A). Interestingly, in compound 2 pre-treated cells membrane fluorescence was largely reduced (FIG. 1: compare panels E and C) indicating that compound 2 precludes binding of TAC-FITC to the membrane androgen receptor. Notably all examined fields contained cells, as indicated by control DAPI stainings (in blue, panels B, D, F).

Taken together these results indicate that compound 2 is capable of binding to the membrane androgen receptor. Furthermore, without wishing to be bound by theory, these results clearly suggest that specific Na⁺K⁺ ATPase inhibitors may function as membrane androgen receptor ligands. Since mAR is functionally implicated in cancer death by apoptosis, it is likely that the anti-cancer action of different Na⁺K⁺ ATPase inhibitors depends not only on their capacity to bind and inhibit Na⁺K⁺ ATPase, but also on their ability to bind to the membrane androgen receptor.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### Incorporation by Reference

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A compound of Formula (I) 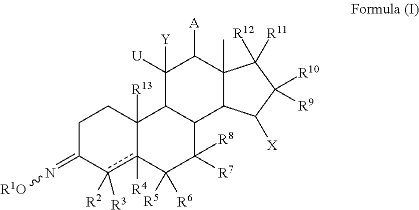
wherein [symbol]
represents a single bond or double bond in the ring structure of Formula (I);
A and X, for each occurrence, independently is H or halogen;
One of U and Y is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸;
or U and Y together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;
R¹ is aminoalkyl, alkyl, or a 4- to 6-membered heterocyclic ring, wherein said aminoalkyl is optionally substituted by one or more alkyl or acetyl; said alkyl, for each occurrence, independently is optionally substituted by guanidinyl, heteroaryl, or a 4- to 6-membered heterocyclic ring; and each 4- to 6-membered heterocyclic ring independently is optionally substituted by one or more alkyl;
one of R² and R³ is H, halogen, optionally substituted (C₁-C₆)alkyl, OH, or absent, and the other is H, halogen, optionally substituted aryl, optionally substituted heteroaryl, (C₁-C₆)alkyl, OH, or absent;
or R² and R³ together with the carbon to which they are attached form cycloalkyl;
R⁴ is H, —OH, or absent;
One of R⁵ and R⁶ is H, OH, or halogen, and the other is H; —OR¹⁴; alkyl optionally substituted with hydroxyl or alkoxy; —C(O)—NH₂; —C(O)—O-alkyl; —NHR¹⁵; alkynyl;
or R⁵ and R⁶ together with the carbon to which they are attached form C═O, C═C(R¹⁶)₂, or C═N—OR²⁰;
One of R⁷ and R⁸ is H or halogen, and the other is H; —OR¹⁴; alkyl optionally substituted with hydroxyl or alkoxy; —C(O)—NH₂; —C(O)—O-alkyl; —NHR¹⁵; alkynyl; or R⁷ and R⁸ together with the carbon to which they are attached form C═O, C═C(R¹⁶)₂, C═N—OR²⁰, or a cyclopropyl ring;
One of R⁹ and R¹⁰ is H, and the other is H, —OH, alkyl, alkenyl, CH₂OR¹⁷, formyl, COOR¹⁷, COR¹⁷, —C≡C—R¹⁷, halogen, or C(O)NR¹⁷R¹⁸;
or R⁹ and R¹⁰ together with the carbon to which they are attached form C═O, C═C(R¹⁶)₂, C═N—OR¹⁷, C═C—C(O)R¹⁷, C═C—C(O)OR¹⁷, C═C—C(O)NR¹⁷R¹⁸, or C═CH—Ar;
One of R¹¹ and R¹² is H, and the other is —OH; alkoxy optionally substituted with a hydroxyl or an amino group; optionally substituted heterocyclyl; optionally substituted heteroaryl; or —OC(O)R¹⁹;
or R¹¹ and R¹² together with the carbon atom to which they are attached form C═O;
R¹³ is H, —CH₂OR¹⁷, —CHO, —COOR¹⁷, —COR¹⁷, —C≡C—R¹⁷, —C(O)NR¹⁷R¹⁸, —(C₁-C₆)alkyl, or a heteroaryl, wherein said (C₁-C₆)alkyl is further optionally substituted by one or more substituents selected from the group of (C₁-C₄)alkyl, hydroxyl, halogen, alkoxy, aryl, —C(O)—NR¹⁷R¹⁸, —OC(O)-alkyl, —C(O)—O-alkyl, cycloalkyl, heteroaryl, and —NR¹⁷R¹⁸; and said heteroaryl is further optionally substituted by (C₁-C₄)alkyl, halogen, hydroxyl, alkoxy, arylalkyl, or cycloalkyl(C₁-C₄)alkyl;
R¹⁴ is H, alkyl, or —NO₂;
R¹⁵ is H or formyl;
R¹⁶, for each occurrence, is the same or different and is H or halogen;
R¹⁷ and R¹⁸, for each occurrence, independently is H, alkyl, alkenyl, alkynyl, cycloalkyl, aryl, aminocarbonyl, alkoxycarbonyl, amino, or halogen; wherein R¹⁷ and R¹⁸ independently are further optionally substituted with one or more moieties selected from the group of alkyl, alkenyl, alkynyl, amino, alkoxy, and cycloalkyl,
R¹⁹ is optionally substituted alkyl, or optionally substituted aryl;
R²⁰ is amino(C₁-C₆)alkyl, (C₁-C₆)alkyl, or a 4- to 6-membered heterocyclic ring, wherein said amino(C₁-C₆)alkyl is optionally substituted by one or more (C₁-C₆)alkyl or acetyl; each heterocyclic ring independently is optionally substituted by one or more (C₁-C₆)alkyl; and each (C₁-C₆)alkyl, independently, is optionally substituted by guanidinyl, heteroaryl, or a 4- to 6-membered heterocyclic ring;
or a tautomer, stereoisomer, Z and/or E isomer, optical isomer, N-oxide, hydrate, solvate, polymorph, pharmaceutically acceptable ester, amide, salt, prodrug, and isotopic derivative thereof;
provided that
when [symbol] represents a single bond, R¹³ is methyl, and R⁹ and R¹⁰ are both hydrogen, one of R² and R³ must be an optionally substituted aryl or optionally substituted heteroaryl; and
when [symbol] represents a double bond and R¹³ is methyl, R⁹ and R¹⁰ cannot be both hydrogen at the same time.

2. The compound of claim 1, wherein A and X are both H, both of U and Y are H, and R¹¹ and R¹² together with the carbon atom to which they are attached form C═O.

3. The compound of claim 1, wherein R¹ is amino(C₁-C₆)alkyl, a 4- to 6-membered heterocyclic ring, or (C₁-C₆)alkyl substituted by a 4- to 6-membered heterocyclic ring.

4. The compound of claim 3, wherein R¹ is aminoethyl, pyrrolidinyl, or piperidinylethyl.

5. The compound of claim 4, wherein [symbol] represents a single bond.

6. The compound of claim 5, wherein R¹³ is unsubstituted (C₁-C₆)alkyl, and R⁴ is H or —OH.

7. The compound of claim 6, wherein one of R⁹ and R¹⁰ is H, and the other is halogen.

8. The compound of claim 7, wherein one of R⁹ and R¹⁰ is H, and the other is F.

9. The compound of claim 8, wherein R⁷ and R⁸ are both H.

10. The compound of claim 9, wherein both of R² and R³ are H.

11-15. (canceled)

16. The compound of claim 10, wherein the compound is selected from the group consisting of:
1) 16-Fluoro-10,13-dimethyl-tetradecahydro-cyclopenta[a]phenanthrene-3,17-dione 3-[O-(2-amino-ethyl)-oxime]: 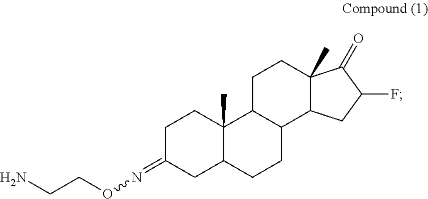 [SMILES: C[C@]12CCC(=NOCCN)CC1CCC1C2CC[C@]2(C)C(=O)C(F)CC12]
2) 16-Fluoro-10,13-dimethyl-tetradecahydro-cyclopenta[a]phenanthrene-3,17-dione 3-(O-pyrrolidin-3-yl-oxime): 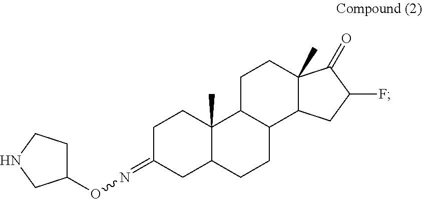 [SMILES: C[C@]12CCC(=NOC3CCNC3)CC1CCC1C2CC[C@]2(C)C(=O)C(F)CC12]
3) 16-Fluoro-10,13-dimethyl-tetradecahydro-cyclopenta[a]phenanthrene-3,6,17-trione 3-[O-(2-amino-ethyl)-oxime]: 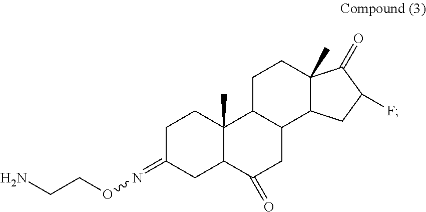 [SMILES: C[C@]12CCC(=NOCCN)CC1C(=O)CC1C2CC[C@]2(C)C(=O)C(F)CC12]
4) 16-Fluoro-10,13-dimethyl-tetradecahydro-cyclopenta[a]phenanthrene-3,6,17-trione 3-(O-pyrrolidin-3-yl-oxime): 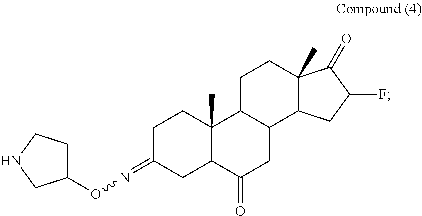 [SMILES: C[C@]12CCC(=NOC3CCNC3)CC1C(=O)CC1C2CC[C@]2(C)C(=O)C(F)CC12]
5) 16-Fluoro-10,13-dimethyl-6-methylene-tetradecahydro-cyclopenta[a]phenanthrene-3,17-dione 3-[O-(2-amino-ethyl)-oxime]: 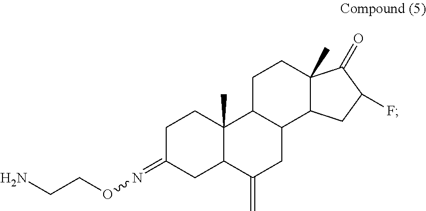 [SMILES: C=C1CC2C(CC[C@]3(C)C(=O)C(F)CC23)[C@@]2(C)CCC(=NOCCN)CC12]
6) 16-Fluoro-10,13-dimethyl-6-methylene-tetradecahydro-cyclopenta[a]phenanthrene-3,17-dione 3-(O-pyrrolidin-3-yl-oxime): 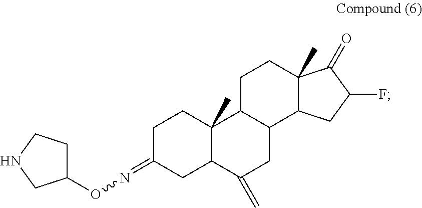 [SMILES: C=C1CC2C(CC[C@]3(C)C(=O)C(F)CC23)[C@@]2(C)CCC(=NOC3CCNC3)CC12]
7) 16-Fluoro-10,13-dimethyl-tetradecahydro-cyclopenta[a]phenanthrene-3,6,17-trione 3-[O-(2-amino-ethyl)-oxime]6-(O-methyl-oxime): 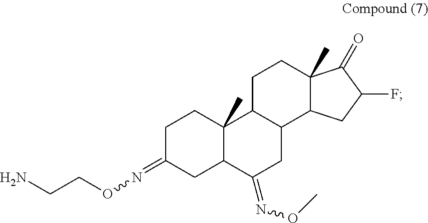 [SMILES: CON=C1CC2C(CC[C@]3(C)C(=O)C(F)CC23)[C@@]2(C)CCC(=NOCCN)CC12]
8) 16-Fluoro-10,13-dimethyl-tetradecahydro-cyclopenta[a]phenanthrene-3,6,17-trione 6-(O-methyl-oxime) 3-(O-pyrrolidin-3-yl-oxime): 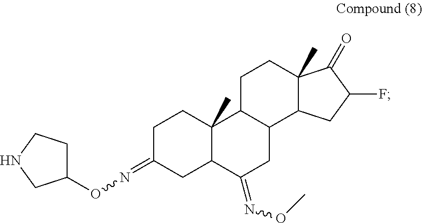 [SMILES: CON=C1CC2C(CC[C@]3(C)C(=O)C(F)CC23)[C@@]2(C)CCC(=NOC3CCNC3)CC12]
16-Fluoro-5-hydroxy-10,13-dimethyl-6-methylene-tetradecahydro-cyclopenta[a]phenanthrene-3,17-dione 3-(O-pyrrolidin-3-yl-oxime): 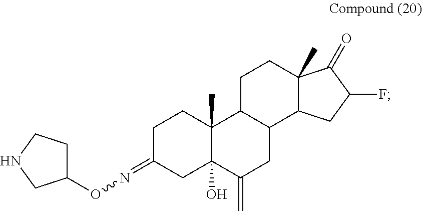 [SMILES: C=C1CC2C3CC(F)C(=O)[C@@]3(C)CCC2[C@@]2(C)CCC(=NOC3CCNC3)C[C@]12O]
16-Fluoro-10,13-dimethyl-tetradecahydro-cyclopenta[a]phenanthrene-3,17-dione 3-[O-(2-piperidin-1-yl-ethyl)-oxime]: 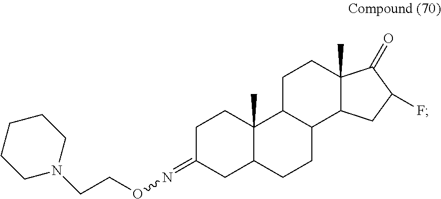 [SMILES: C[C@]12CCC(=NOCCN3CCCCC3)CC1CCC1C2CC[C@]2(C)C(=O)C(F)CC12]
16-Fluoro-10,13-dimethyl-6-methylene-tetradecahydro-cyclopenta[a]phenanthrene-3,17-dione 3-[O-(2-piperidin-1-yl-ethyl)-oxime]: 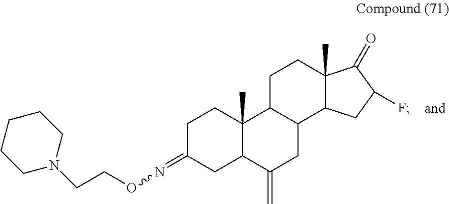 [SMILES: C=C1CC2C(CC[C@]3(C)C(=O)C(F)CC23)[C@@]2(C)CCC(=NOCCN3CCCCC3)CC12 ; "and"]
16-Fluoro-10,13-dimethyl-tetradecahydro-cyclopenta[a]phenanthrene-3,6,17-trione 6-(O-methyl-oxime) 3-[O-(2-piperidin-1-yl-ethyl)-oxime]:

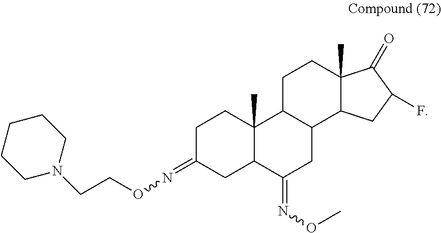 [SMILES: CON=C1CC2C(CC[C@]3(C)C(=O)C(F)CC23)[C@@]2(C)CCC(=NOCCN3CCCCC3)CC12]

17-21. (canceled)

22. The compound of claim 9, wherein the compound is selected from the group of
4-Ethyl-16-fluoro-10,13-dimethyl-tetradecahydro-cyclopenta[a]phenanthrene-3,17-dione 3-(O-pyrrolidin-3-yl-oxime): 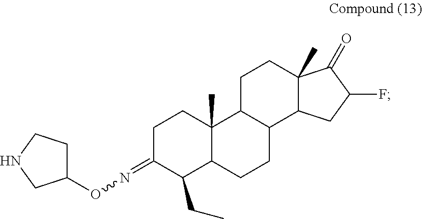 [SMILES: CC[C@H]1C(=NOC2CCNC2)CC[C@]2(C)C3CC[C@]4(C)C(=O)C(F)CC4C3CCC12]
16-Fluoro-10,13-dimethyl-4-phenyl-tetradecahydro-cyclopenta[a]phenanthrene-3,17-dione 3-(O-pyrrolidin-3-yl-oxime): 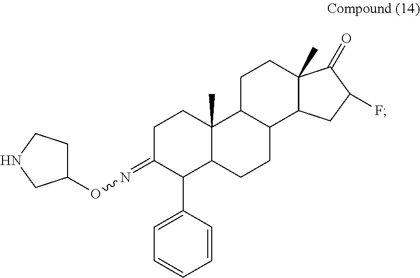 [SMILES: C[C@]12CCC(=NOC3CCNC3)C(c3ccccc3)C1CCC1C2CC[C@]2(C)C(=O)C(F)CC12]
4-Ethyl-16-fluoro-10,13-dimethyl-tetradecahydro-cyclopenta[a]phenanthrene-3,6,17-trione 3-(O-pyrrolidin-3-yl-oxime): 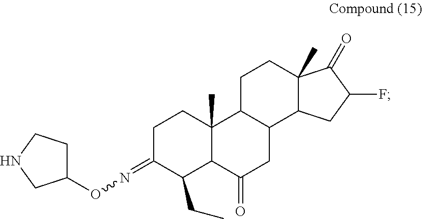 [SMILES: CC[C@H]1C(=NOC2CCNC2)CC[C@]2(C)C3CC[C@]4(C)C(=O)C(F)CC4C3CC(=O)C12]
4-Ethyl-16-fluoro-10,13-dimethyl-6-methylene-tetradecahydro-cyclopenta[a]phenanthrene-3,17-dione 3-(O-pyrrolidin-3-yl-oxime): 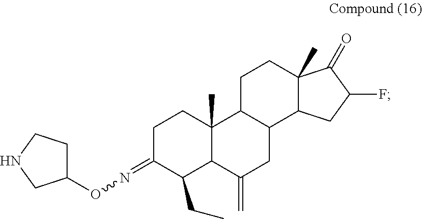 [SMILES: C=C1CC2C(CC[C@]3(C)C(=O)C(F)CC23)[C@@]2(C)CCC(=NOC3CCNC3)[C@H](CC)C12]
4-Ethyl-16-fluoro-10,13-dimethyl-tetradecahydro-cyclopenta[a]phenanthrene-3,6,17-trione 6-(O-methyl-oxime) 3-(O-pyrrolidin-3-yl-oxime: 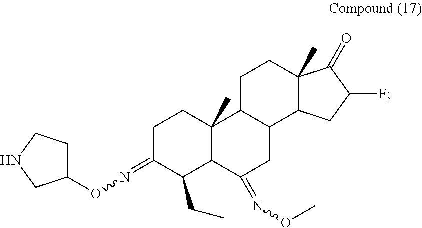 [SMILES: CC[C@H]1C(=NOC2CCNC2)CC[C@]2(C)C3CC[C@]4(C)C(=O)C(F)CC4C3CC(=NOC)C12]
16-Fluoro-10,13-dimethyl-6-methylene-4-phenyl-tetradecahydro-cyclopenta[a]phenanthrene-3,17-dione 3-(O-pyrrolidin-3-yl-oxime): 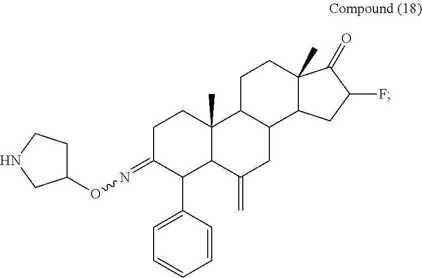 [SMILES: C=C1CC2C(CC[C@]3(C)C(=O)C(F)CC23)[C@@]2(C)CCC(=NOC3CCNC3)C(c3ccccc3)C12]
and
16-Fluoro-10,13-dimethyl-4-phenyl-tetradecahydro-cyclopenta[a]phenanthrene-3,6,17-trione 6-(O-methyl-oxime) 3-(O-pyrrolidin-3-yl-oxime):

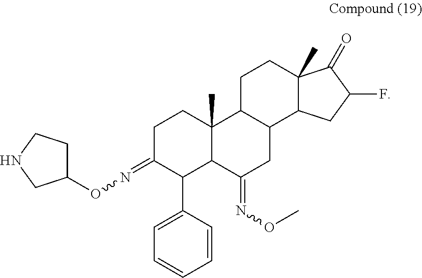 [SMILES: CON=C1CC2C(CC[C@]3(C)C(=O)C(F)CC23)[C@@]2(C)CCC(=NOC3CCNC3)C(c3ccccc3)C12]

23-25. (canceled)

26. The compound of claim 8, wherein the compound is selected from the group consisting of:
16-Fluoro-10,13-dimethyl-7-methylene-tetradecahydro-cyclopenta[a]phenanthrene-3,17-dione 3-(O-pyrrolidin-3-yl-oxime): 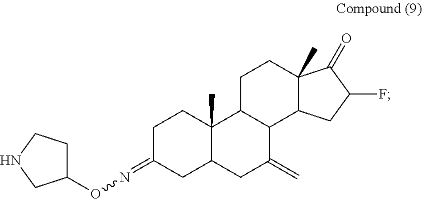 [SMILES: C=C1CC2CC(=NOC3CCNC3)CC[C@]2(C)C2CC[C@]3(C)C(=O)C(F)CC3C12]
7-Difluoromethylene-16-fluoro-10,13-dimethyl-tetradecahydro-cyclopenta[a]phenanthrene-3,17-dione 3-(O-pyrrolidin-3-yl-oxime): 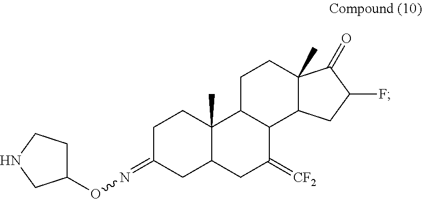
16-Fluoro-6-hydroxymethyl-10,13-dimethyl-dodecahydro-cyclopenta[a]phenanthrene-3,7,17-trione 3-[O-(2-amino-ethyl)-oxime]: 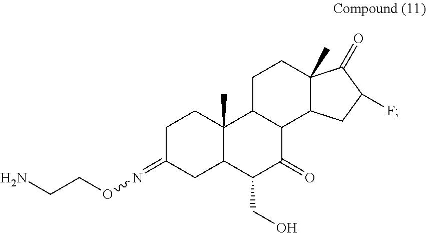 [SMILES: C[C@]12CCC(=NOCCN)CC1[C@@H](CO)C(=O)C1C2CC[C@]2(C)C(=O)C(F)CC12]
16-Fluoro-6-hydroxymethyl-10,13-dimethyl-dodecahydro-cyclopenta[a]phenanthrene-3,7,17-trione 3-(O-pyrrolidin-3-yl-oxime): 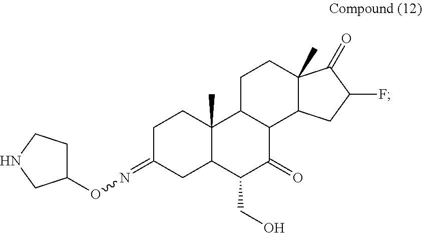 [SMILES: C[C@]12CCC(=NOC3CCNC3)CC1[C@@H](CO)C(=O)C1C2CC[C@]2(C)C(=O)C(F)CC12]
16-Fluoro-7-hydroxy-6-hydroxymethyl-10,13-dimethyl-tetradecahydro-cyclopenta[a]phenanthrene-3,17-dione 3-(O-pyrrolidin-3-yl-oxime):

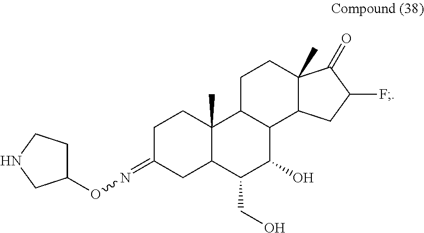 [SMILES: C[C@]12CCC(=NOC3CCNC3)CC1[C@@H](CO)[C@@H](O)C1C2CC[C@]2(C)C(=O)C(F)CC12]

27-37. (canceled)

38. The compound of claim 6, wherein the compound is selected from the group consisting of
10,13-Dimethyl-4-phenyl-tetradecahydro-cyclopenta[a]phenanthrene-3,17-dione 3-(O-pyrrolidin-3-yl-oxime): 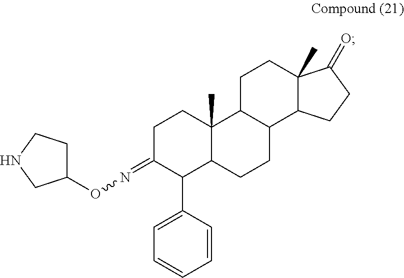 [SMILES: C[C@]12CCC(=NOC3CCNC3)C(c3ccccc3)C1CCC1C2CC[C@]2(C)C(=O)CCC12]
10,13-Dimethyl-6-methylene-4-phenyl-tetradecahydro-cyclopenta[a]phenanthrene-3,17-dione 3-(O-pyrrolidin-3-yl-oxime): 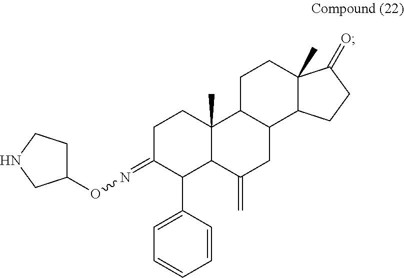 [SMILES: C=C1CC2C(CC[C@]3(C)C(=O)CCC23)[C@@]2(C)CCC(=NOC3CCNC3)C(c3ccccc3)C12]
and
10,13-Dimethyl-4-phenyl-tetradecahydro-cyclopenta[a]phenanthrene-3,6,17-trione 6-(O-methyl-oxime) 3-(O-pyrrolidin-3-yl-oxime):

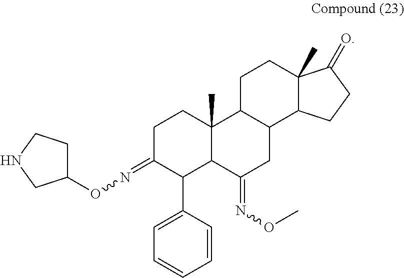 [SMILES: CON=C1CC2C(CC[C@]3(C)C(=O)CCC23)[C@@]2(C)CCC(=NOC3CCNC3)C(c3ccccc3)C12]

39-47. (canceled)

48. The compound of claim 5, wherein the compound is selected from the group of
10-Hydroxymethyl-13-methyl-tetradecahydro-cyclopenta[a]phenanthrene-3,17-dione 3-[O-(2-amino-ethyl)-oxime]: 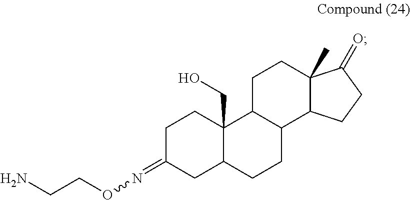 [SMILES: C[C@]12CCC3C(CCC4CC(=NOCCN)CC[C@@]43CO)C1CCC2=O]
10-Hydroxymethyl-13-methyl-tetradecahydro-cyclopenta[a]phenanthrene-3,17-dione 3-(O-pyrrolidin-3-yl-oxime): 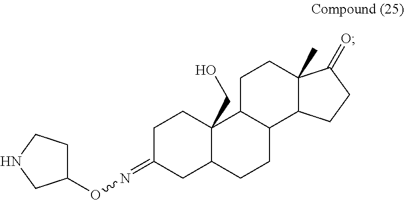 [SMILES: C[C@]12CCC3C(CCC4CC(=NOC5CCNC5)CC[C@@]43CO)C1CCC2=O]
4-Ethyl-10-hydroxymethyl-13-methyl-tetradecahydro-cyclopenta[a]phenanthrene-3,17-dione 3-(O-pyrrolidin-3-yl-oxime): 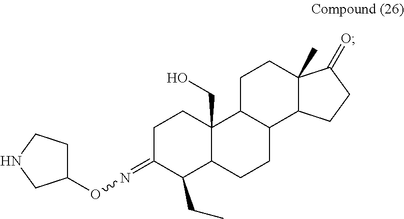 [SMILES: CC[C@H]1C(=NOC2CCNC2)CC[C@]2(CO)C3CC[C@]4(C)C(=O)CCC4C3CCC12]
10-Hydroxymethyl-13-methyl-4-phenyl-tetradecahydro-cyclopenta[a]phenanthrene-3,17-dione 3-(O-pyrrolidin-3-yl-oxime): 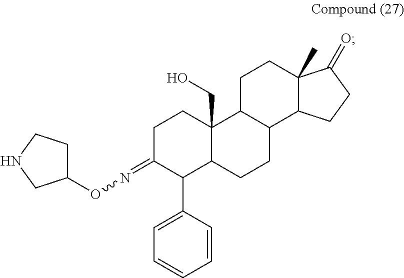 [SMILES: C[C@]12CCC3C(CCC4C(c5ccccc5)C(=NOC5CCNC5)CC[C@@]43CO)C1CCC2=O]
10-Hydroxymethyl-13-methyl-tetradecahydro-cyclopenta[a]phenanthrene-3,6,17-trione 3-(O-pyrrolidin-3-yl-oxime): 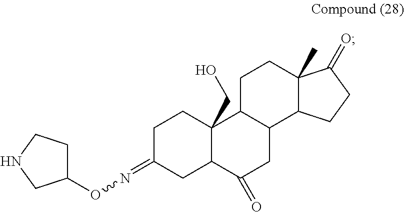 [SMILES: C[C@]12CCC3C(CC(=O)C4CC(=NOC5CCNC5)CC[C@@]43CO)C1CCC2=O]
10-Hydroxymethyl-13-methyl-6-methylene-tetradecahydro-cyclopenta[a]phenanthrene-3,17-dione 3-(O-pyrrolidin-3-yl-oxime): 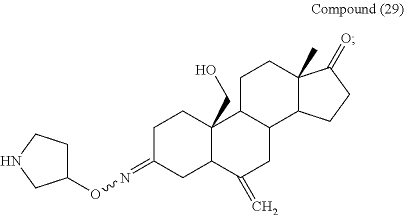 [SMILES: C=C1CC2C(CC[C@]3(C)C(=O)CCC23)[C@@]2(CO)CCC(=NOC3CCNC3)CC12]
10-Hydroxymethyl-13-methyl-tetradecahydro-cyclopenta[a]phenanthrene-3,6,17-trione 6-(O-methyl-oxime) 3-(O-pyrrolidin-3-yl-oxime): 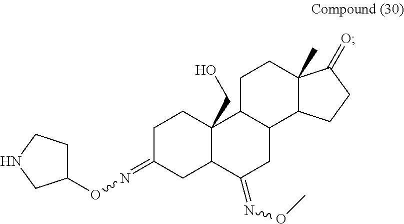 [SMILES: CON=C1CC2C(CC[C@]3(C)C(=O)CCC23)[C@@]2(CO)CCC(=NOC3CCNC3)CC12]
4-Ethyl-10-hydroxymethyl-13-methyl-tetradecahydro-cyclopenta[a]phenanthrene-3,6,17-trione 3-(O-pyrrolidin-3-yl-oxime): 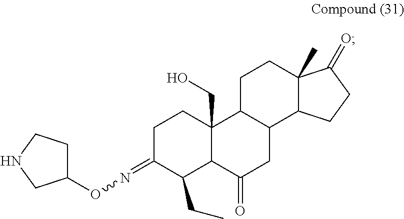 [SMILES: CC[C@H]1C(=NOC2CCNC2)CC[C@]2(CO)C3CC[C@]4(C)C(=O)CCC4C3CC(=O)C12]
4-Ethyl-10-hydroxymethyl-13-methyl-6-methylene-tetradecahydro-cyclopenta[a]phenanthrene-3,17-dione 3-(O-pyrrolidin-3-yl-oxime): 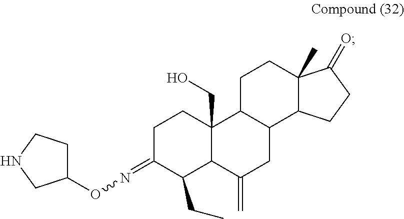 [SMILES: C=C1CC2C(CC[C@]3(C)C(=O)CCC23)[C@@]2(CO)CCC(=NOC3CCNC3)[C@H](CC)C12]
4-Ethyl-10-hydroxymethyl-13-methyl-tetradecahydro-cyclopenta[a]phenanthrene-3,6,17-trione 6-(O-methyl-oxime) 3-(O-pyrrolidin-3-yl-oxime): 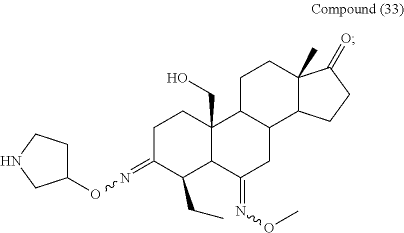 [SMILES: CC[C@H]1C(=NOC2CCNC2)CC[C@]2(CO)C3CC[C@]4(C)C(=O)CCC4C3CC(=NOC)C12]
5-Hydroxy-10-hydroxymethyl-13-methyl-6-methylene-tetradecahydro-cyclopenta[a]phenanthrene-3,17-dione 3-(O-pyrrolidin-3-yl-oxime): 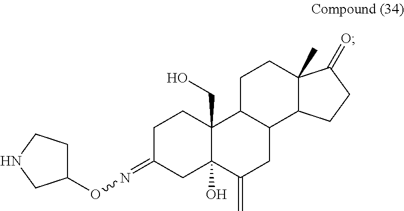 [SMILES: C=C1CC2C(CC[C@]3(C)C(=O)CCC23)[C@@]2(CO)CCC(=NOC3CCNC3)C[C@]12O]
10-Hydroxymethyl-13-methyl-7-methylene-tetradecahydro-cyclopenta[a]phenanthrene-3,17-dione 3-(O-pyrrolidin-3-yl-oxime): 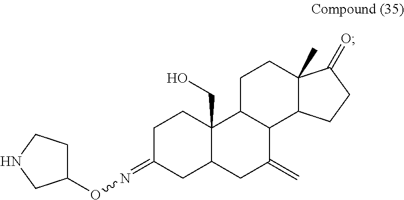 [SMILES: C=C1CC2CC(=NOC3CCNC3)CC[C@]2(CO)C2CC[C@]3(C)C(=O)CCC3C12]
7-Difluoromethylene-10-hydroxymethyl-13-methyl-tetradecahydro-cyclopenta[a]phenanthrene-3,17-dione 3-(O-pyrrolidin-3-yl-oxime): 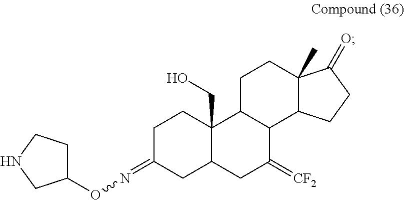
6,10-Bis-hydroxymethyl-13-methyl-dodecahydro-cyclopenta[a]phenanthrene-3,7,17-trione 3-(O-pyrrolidin-3-yl-oxime): 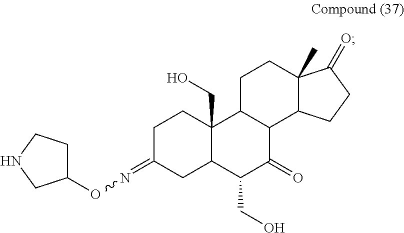 [SMILES: C[C@]12CCC3C(C(=O)[C@H](CO)C4CC(=NOC5CCNC5)CC[C@]34CO)C1CCC2=O]
and
10-Hydroxymethyl-13-methyl-7-methylene-tetradecahydro-cyclopenta[a]phenanthrene-3,17-dione 3-(O-pyrrolidin-3-yl-oxime): 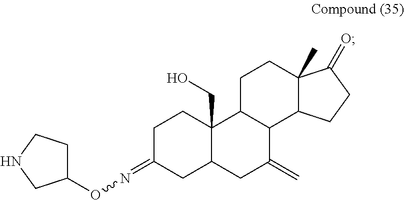 [SMILES: C=C1CC2CC(=NOC3CCNC3)CC[C@]2(CO)C2CC[C@]3(C)C(=O)CCC3C12]
7-Difluoromethylene-10-hydroxymethyl-13-methyl-tetradecahydro-cyclopenta[a]phenanthrene-3,17-dione 3-(O-pyrrolidin-3-yl-oxime): 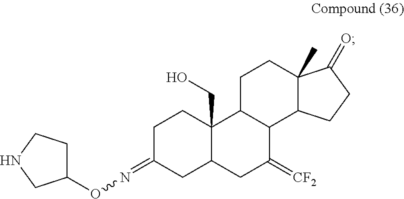
6,10-Bis-hydroxymethyl-13-methyl-dodecahydro-cyclopenta[a]phenanthrene-3,7,17-trione 3-(O-pyrrolidin-3-yl-oxime): 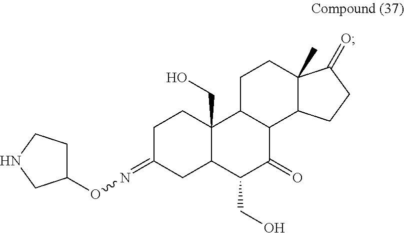 [SMILES: C[C@]12CCC3C(C(=O)[C@H](CO)C4CC(=NOC5CCNC5)CC[C@]34CO)C1CCC2=O]
7-Hydroxy-6,1-bis-hydroxymethyl-13-methyl-tetradecahydro-cyclopenta[a]phenanthrene-3,17-dione 3-(O-pyrrolidin-3-yl-oxime): 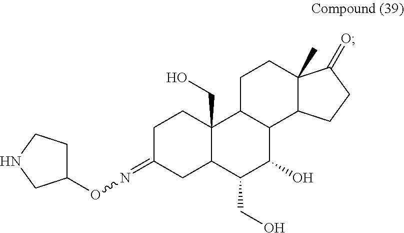 [SMILES: C[C@]12CCC3C(C1CCC2=O)[C@H](O)[C@H](CO)C1CC(=NOC2CCNC2)CC[C@]31CO]
Acetic acid 13-methyl-6-methylene-17-oxo-3-(pyrrolidin-3-yloxyimino)-hexadecahydro-cyclopenta[a]phenanthren-10-ylmethyl ester: 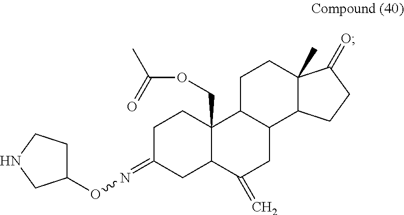 [SMILES: C=C1CC2C(CC[C@]3(C)C(=O)CCC23)[C@@]2(COC(C)=O)CCC(=NOC3CCNC3)CC12]
Acetic acid 6-methoxyimino-13-methyl-17-oxo-3-(pyrrolidin-3-yloxyimino)-hexadecahydro-cyclopenta[a]phenanthren-10-ylmethyl ester: 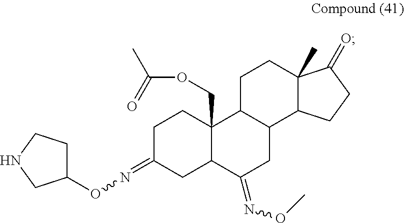 [SMILES: CON=C1CC2C(CC[C@]3(C)C(=O)CCC23)[C@@]2(COC(C)=O)CCC(=NOC3CCNC3)CC12]
Acetic acid 4-ethyl-13-methyl-6-methylene-17-oxo-3-(pyrrolidin-3-yloxyimino)-hexadecahydro-cyclopenta[a]phenanthren-10-ylmethyl ester: 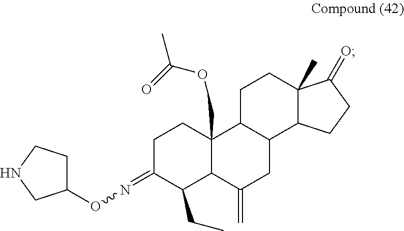 [SMILES: C=C1CC2C(CC[C@]3(C)C(=O)CCC23)[C@@]2(COC(C)=O)CCC(=NOC3CCNC3)[C@H](CC)C12]
Acetic acid 4-ethyl-6-methoxyimino-13-methyl-17-oxo-3-(pyrrolidin-3-yloxyimino)-hexadecahydro-cyclopenta[a]phenanthren-10-ylmethyl ester: 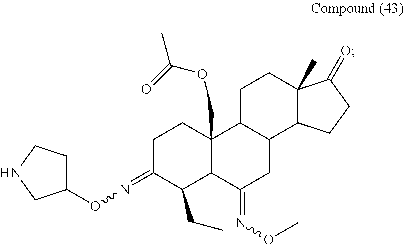 [SMILES: CC[C@H]1C(=NOC2CCNC2)CC[C@]2(COC(C)=O)C3CC[C@]4(C)C(=O)CCC4C3CC(=NOC)C12]
Acetic acid 13-methyl-6-methylene-17-oxo-4-phenyl-3-(pyrrolidin-3-yloxyimino)-hexadecahydro-cyclopenta[a]phenanthren-10-ylmethyl ester: 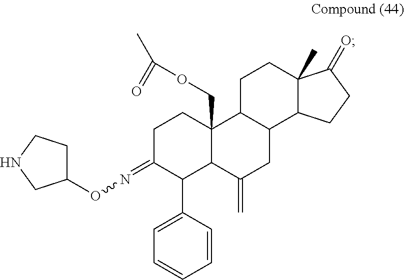 [SMILES: C=C1CC2C(CC[C@]3(C)C(=O)CCC23)[C@@]2(COC(C)=O)CCC(=NOC3CCNC3)C(c3ccccc3)C12]
Acetic acid 6-methoxyimino-13-methyl-17-oxo-4-phenyl-3-(pyrrolidin-3-yloxyimino)-hexadecahydro-cyclopenta[a]phenanthren-10-ylmethyl ester: 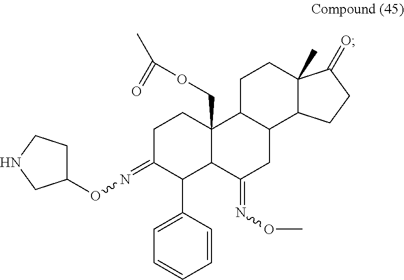 [SMILES: CON=C1CC2C(CC[C@]3(C)C(=O)CCC23)[C@@]2(COC(C)=O)CCC(=NOC3CCNC3)C(c3ccccc3)C12]
Acetic acid 6-hydroxymethyl-13-methyl-7,17-dioxo-3-(pyrrolidin-3-yloxyimino)-hexadecahydro-cyclopenta[a]phenanthren-10-ylmethyl ester: 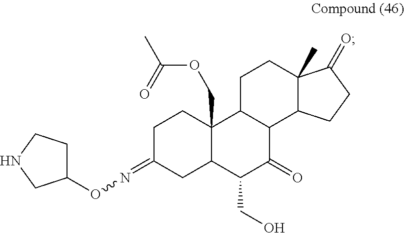 [SMILES: CC(=O)OC[C@]12CCC(=NOC3CCNC3)CC1[C@@H](CO)C(=O)C1C2CC[C@]2(C)C(=O)CCC12]
Acetic acid 7-hydroxy-6-hydroxymethyl-13-methyl-17-oxo-3-(pyrrolidin-3-yloxyimino)-hexadecahydro-cyclopenta[a]phenanthren-10-ylmethyl ester: 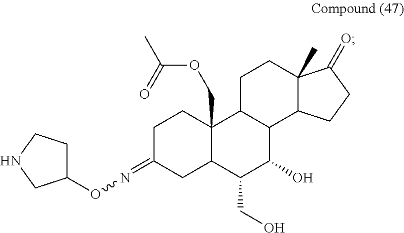 [SMILES: CC(=O)OC[C@]12CCC(=NOC3CCNC3)CC1[C@@H](CO)[C@@H](O)C1C2CC[C@]2(C)C(=O)CCC12]
and
Acetic acid 13-methyl-17-oxo-3-(pyrrolidin-3-yloxyimino)-hexadecahydro-cyclopenta[a]phenanthren-10-ylmethyl ester:

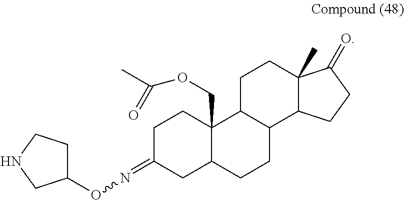 [SMILES: CC(=O)OC[C@]12CCC(=NOC3CCNC3)CC1CCC1C2CC[C@]2(C)C(=O)CCC12]

49-65. (canceled)

66. The compound of claim 4, wherein [symbol] represents a double bond.

67-75. (canceled)

76. The compound of claim 66, wherein the compound is selected from the group consisting of:
16-Fluoro-10,13-dimethyl-1,6,7,8,9,10,11,12,13,14,15,16-dodecahydro-2H-cyclopenta[a]phenanthrene-3,17-dione 3-(O-pyrrolidin-3-yl-oxime) 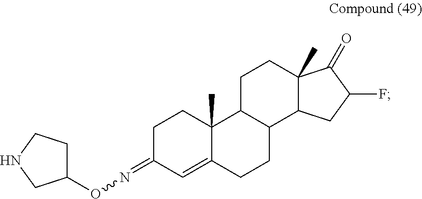 [SMILES: C[C@]12CCC(=NOC3CCNC3)C=C1CCC1C2CC[C@]2(C)C(=O)C(F)CC12]
16-Fluoro-10,13-dimethyl-6-methylene-1,6,7,8,9,10,11,12,13,14,15,16-dodecahydro-2H-cyclopenta[a]phenanthrene-3,17-dione 3-(O-pyrrolidin-3-yl-oxime): 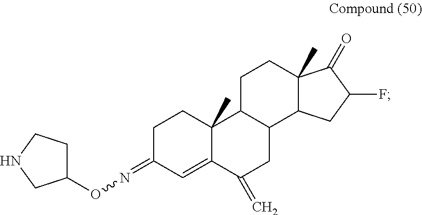 [SMILES: C=C1CC2C(CC[C@]3(C)C(=O)C(F)CC23)[C@@]2(C)CCC(=NOC3CCNC3)C=C12]
6-Ethylimino-16-fluoro-10,13-dimethyl-1,6,7,8,9,10,11,12,13,14,15,16-dodecahydro-2H-cyclopenta[a]phenanthrene-3,17-dione 3-(O-pyrrolidin-3-yl-oxime): 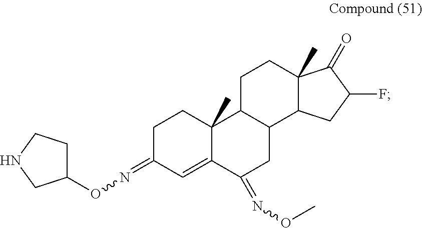 [SMILES: CON=C1CC2C(CC[C@]3(C)C(=O)C(F)CC23)[C@@]2(C)CCC(=NOC3CCNC3)C=C12]
16-Fluoro-7-hydroxy-6-hydroxymethyl-10,13-dimethyl-1,6,7,8,9,10,11,12,13,14,15,16-dodecahydro-2H-cyclopenta[a]phenanthrene-3,17-dione 3-(O-pyrrolidin-3-yl-oxime): 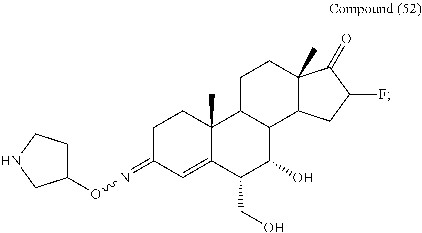 [SMILES: C[C@]12CCC(=NOC3CCNC3)C=C1[C@@H](CO)[C@@H](O)C1C2CC[C@]2(C)C(=O)C(F)CC12]
10-Hydroxymethyl-13-methyl-1,6,7,8,9,10,11,12,13,14,15,16-dodecahydro-2H-cyclopenta[a]phenanthrene-3,17-dione 3-(O-pyrrolidin-3-yl-oxime): 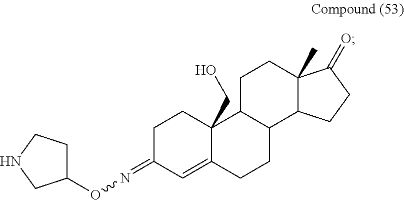 [SMILES: C[C@]12CCC3C(CCC4=CC(=NOC5CCNC5)CC[C@@]43CO)C1CCC2=O]
Acetic acid 13-methyl-17-oxo-3-(pyrrolidin-3-yloxyimino)-1,2,3,6,7,8,9,11,12,13,14,15,16,17-tetradecahydro-cyclopenta[a]phenanthren-10-ylmethyl ester: 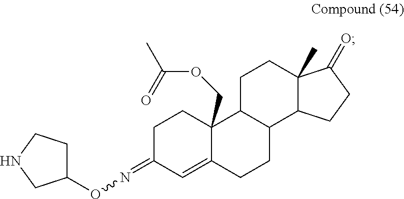 [SMILES: CC(=O)OC[C@]12CCC(=NOC3CCNC3)C=C1CCC1C2CC[C@]2(C)C(=O)CCC12]
10-Hydroxymethyl-13-methyl-6-methylene-1,6,7,8,9,10,11,12,13,14,15,16-dodecahydro-2H-cyclopenta[a]phenanthrene-3,17-dione 3-(O-pyrrolidin-3-yl-oxime): 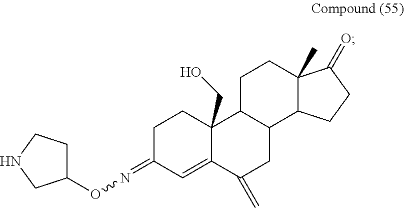 [SMILES: C=C1CC2C(CC[C@]3(C)C(=O)CCC23)[C@@]2(CO)CCC(=NOC3CCNC3)C=C12]
6-Ethylimino-10-hydroxymethyl-13-methyl-1,6,7,8,9,10,11,12,13,14,15,16-dodecahydro-2H-cyclopenta[a]phenanthrene-3,17-dione 3-(O-pyrrolidin-3-yl-oxime): 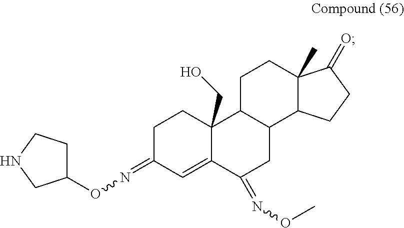 [SMILES: CON=C1CC2C(CC[C@]3(C)C(=O)CCC23)[C@@]2(CO)CCC(=NOC3CCNC3)C=C12]
7-Hydroxy-6,10-bis-hydroxymethyl-13-methyl-1,6,7,8,9,10,11,12,13,14,15,16-dodecahydro-2H-cyclopenta[a]phenanthrene-3,17-dione 3-(O-pyrrolidin-3-yl-oxime): 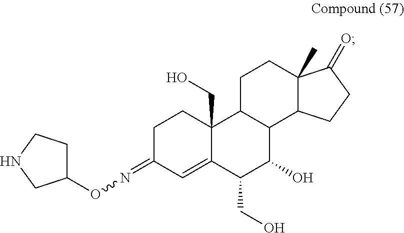 [SMILES: C[C@]12CCC3C(C1CCC2=O)[C@H](O)[C@H](CO)C1=CC(=NOC2CCNC2)CC[C@@]13CO]
16-Fluoro-10,13-dimethyl-6-methylene-1,6,7,8,9,10,11,12,13,14,15,16-dodecahydro-2H-cyclopenta[a]phenanthrene-3,17-dione 3-[O-(2-piperidin-1-yl-ethyl)-oxime]: 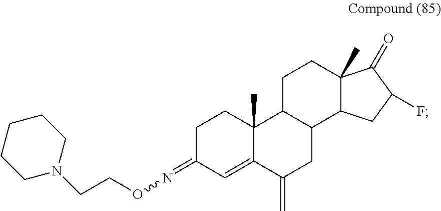 [SMILES: C=C1CC2C(CC[C@]3(C)C(=O)C(F)CC23)[C@@]2(C)CCC(=NOCCN3CCCCC3)C=C12]
and
16-Fluoro-6,10,13-trimethyl-1,6,7,8,9,10,11,12,13,14,15,16-dodecahydro-2H-cyclopenta[a]phenanthrene-3,17-dione 3-(O-pyrrolidin-3-yl-oxime):

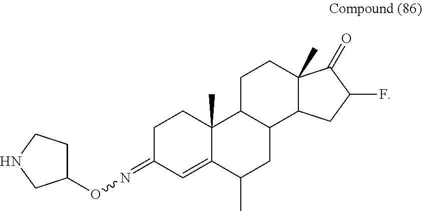 [SMILES: CC1CC2C(CC[C@]3(C)C(=O)C(F)CC23)[C@@]2(C)CCC(=NOC3CCNC3)C=C12]

77. The compound of claim 1, wherein said compound is Formula (Ia): 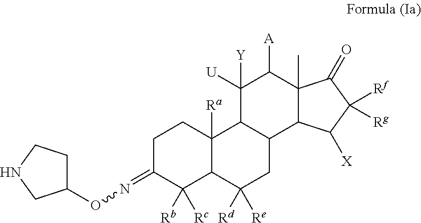
wherein:
A and X, for each occurrence, independently is H or halogen;
One of U and Y is H, and the other is H, or halogen; or U and Y together with the carbon to which they are attached form C═O;
R^{a} is H, or (C₁-C₆)alkyl that is further optionally substituted by hydroxyl or —OC(O)—(C₁-C₆)alkyl;
one of R^{b} and R^{c} is H, or halogen, and the other is H, halogen, optionally substituted aryl, optionally substituted heteroaryl, alkyl, or OH;
One of R^{d} and R^{e} is H or OH, and the other is H; or —OH; or R^{d} and R^{e} together with the carbon to which they are attached form C═O, C═C(R)₂, or C═N—OR^{k};
One of R^{f} and R^{g} is H, and the other is H or halogen;
R^{j} is the same or different on each occurrence and is H or halogen;
R^{k} is amino(C₁-C₆)alkyl or (C₁-C₆)alkyl;
or a tautomer, stereoisomer, Z and/or E isomer, optical isomer, N-oxide, hydrate, solvate, polymorph, pharmaceutically acceptable ester, amide, salt, prodrug, and isotopic derivative thereof;
provided that
when R^{a} is methyl and R^{f} and R^{g} are both hydrogen, one of R^{b} and R^{c} must be an optionally substituted aryl or optionally substituted heteroaryl.

78-91. (canceled)

92. The compound of claim 77, wherein the compound is selected from the group consisting of:
16-Fluoro-10,13-dimethyl-tetradecahydro-cyclopenta[a]phenanthrene-3,17-dione 3-(O-pyrrolidin-3-yl-oxime): 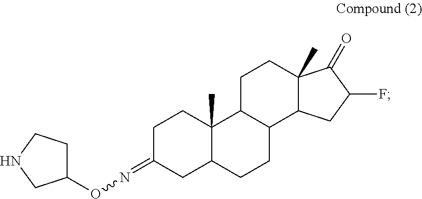 [SMILES: C[C@]12CCC(=NOC3CCNC3)CC1CCC1C2CC[C@]2(C)C(=O)C(F)CC12]
16-Fluoro-10,13-dimethyl-tetradecahydro-cyclopenta[a]phenanthrene-3,6,17-trione 3-(O-pyrrolidin-3-yl-oxime): 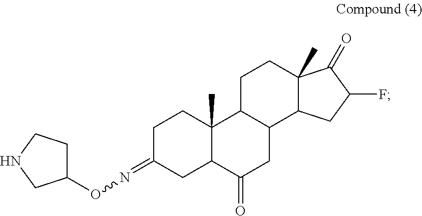 [SMILES: C[C@]12CCC(=NOC3CCNC3)CC1C(=O)CC1C2CC[C@]2(C)C(=O)C(F)CC12]
16-Fluoro-10,13-dimethyl-6-methylene-tetradecahydro-cyclopenta[a]phenanthrene-3,17-dione 3-(O-pyrrolidin-1-yl-oxime) 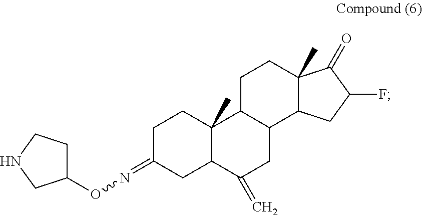 [SMILES: C=C1CC2C(CC[C@]3(C)C(=O)C(F)CC23)[C@@]2(C)CCC(=NOC3CCNC3)CC12]
16-Fluoro-10,13-dimethyl-tetradecahydro-cyclopenta[a]phenanthrene-3,6,17-trione 6-(O-methyl-oxime) 3-(O-pyrrolidin-3-yl-oxime): 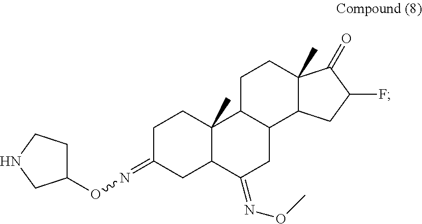 [SMILES: CO/N=C1\CC2C(CC[C@]3(C)C(=O)C(F)CC23)[C@@]2(C)CCC(=NOC3CCNC3)CC12]
10,13-Dimethyl-4-phenyl-tetradecahydro-cyclopenta[a]phenanthrene-3,17-dione 3-(O-pyrrolidin-3-yl-oxime): 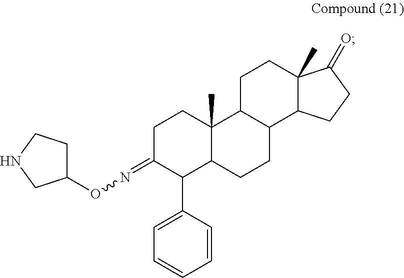 [SMILES: C[C@]12CCC(=NOC3CCNC3)C(c3ccccc3)C1CCC1C2CC[C@]2(C)C(=O)CCC12]
10-Hydroxymethyl-13-methyl-tetradecahydro-cyclopenta[a]phenanthrene-3,17-dione 3-(O-pyrrolidin-3-yl-oxime): 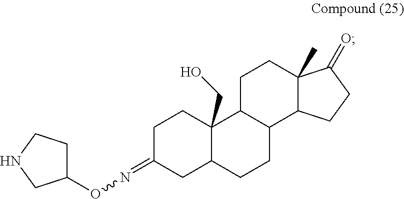 [SMILES: C[C@]12CCC3C(CCC4CC(=NOC5CCNC5)CC[C@@]43CO)C1CCC2=O]
Acetic acid 13-methyl-17-oxo-3-(pyrrolidin-3-yloxyimino)-hexadecahydro-cyclopenta[a]phenanthren-10-ylmethyl ester: 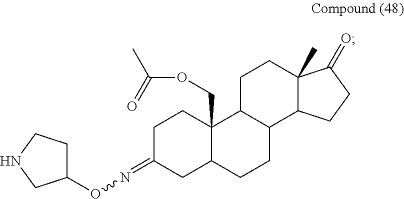 [SMILES: CC(=O)OC[C@]12CCC(=NOC3CCNC3)CC1CCC1C2CC[C@]2(C)C(=O)CCC12]
10-Hydroxymethyl-13-methyl-tetradecahydro-cyclopenta[a]phenanthrene-3,6,17-trione 3-(O-pyrrolidin-3-yl-oxime): 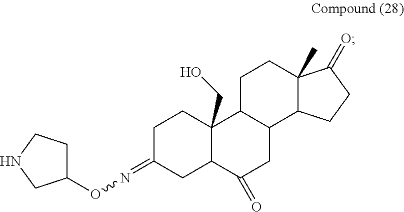 [SMILES: C[C@]12CCC3C(CC(=O)C4CC(=NOC5CCNC5)CC[C@@]43CO)C1CCC2=O]
and
10-Hydroxymethyl-13-methyl-tetradecahydro-cyclopenta[a]phenanthrene-3,6,17-trione 6-(O-methyl-oxime) 3-(O-pyrrolidin-3-yl-oxime):

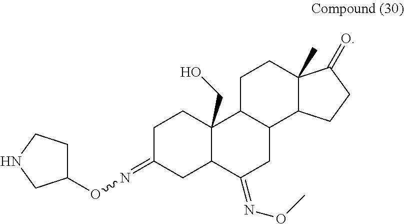 [SMILES: CO/N=C1\CC2C(CC[C@]3(C)C(=O)CCC23)[C@@]2(CO)CCC(=NOC3CCNC3)CC12]

93. A method for reducing the growth, proliferation or survival of a neoplastic cell, the method comprising contacting the cell with an effective amount of a compound of claim 1, thereby reducing the growth, proliferation or survival of a neoplastic cell.

94-97. (canceled)

98. A method of inducing cell death in a neoplastic cell, the method comprising contacting the cell with a therapeutically effective amount of a compound of claim 1, thereby inducing cell death.

99. (canceled)

100. (canceled)

101. A method of preventing or treating a neoplasia in a subject, the method comprising
administering to a subject in need thereof a therapeutically effective amount of a compound of claim 1, thereby preventing or treating neoplasia in a subject.

102-118. (canceled)

119. A pharmaceutical composition comprising a therapeutically effective amount of a compound of claim 1 and a pharmaceutically acceptable excipient or carrier.

120-121. (canceled)

122. A packaged pharmaceutical comprising a therapeutically effective amount of a compound of claim 1, and written instructions for administration of the compound.

123-145. (canceled)
